(19) United States
(12) Patent Application Publication
(10) Pub. No.: US 2023/0226257 A1
(43) Pub. Date: Jul. 20, 2023

(54) DECELLULARIZED TENDON MATRIX METHODS AND USES THEREOF

(71) Applicant: THE STEADMAN CLINIC AND STEADMAN PHILIPPON RESEARCH INSTITUTE, Vail, CO (US)

(72) Inventors: Chelsea S. BAHNEY, Edwards, CO (US); David BERNHOLT, Avon, CO (US); Anna-Laura NELSON, Leadville, CO (US); Peter J. MILLETT, Edwards, CO (US)

(73) Assignee: THE STEADMAN CLINIC AND STEADMAN PHILIPPON RESEARCH INSTITUTE, Vail, CO (US)

(21) Appl. No.: 18/002,514

(22) PCT Filed: Jun. 20, 2021
(86) PCT No.: PCT/US2021/038165

§ 371 Date: Dec. 20, 2022

Related U.S. Application Data
(60) Provisional application No. 63/041,877, filed on Jun. 20, 2020.
(60) Provisional application No. 63/043,931, filed on Jun. 25, 2020.

(51) Int. Cl. A61L 27/36

(52) U.S. Cl. CPC A61L 27/3662; A61L 27/3687; A61L 2430/10

(57) ABSTRACT

Methods of making decellularized tendon matrix (DTM) and DTM hydrogels are provided. These compositions and hydrogels are useful for repairing tendon injuries and in some cases may be used by injection, arthroscopic procedures, or as adjuncts to traditional surgical repair.

### CROSS-REFERENCE TO RELATED APPLICATIONS

This application is an international application claiming the benefit of U.S. Provisional Application No. 63/041,877, filed on Jun. 20, 2020, and U.S. Provisional Application No. 63/043,931, filed on Jun. 25, 2020, each of which is incorporated herein by reference in its entirety.

### FIELD

The disclosure described herein relates generally to decellularized tendon matrix, and methods of making and using decellularized tendon matrix and.

### BACKGROUND

Regenerative medicine is an emerging discipline that has identified many uses for extracellular matrix materials. Tendons are fibrous connective tissues that connect muscle to bone. The connection between muscle and tendon is referred to as the myotendinous junction or as the tendon-muscle insertion point; the connection between tendon and bone is referred to as the osteotendinous junction. This is also known as the tendon insertion or the enthesis, and disease here is known as enthesopathy. This latter connection, the junction between tendon and bone, where tendon collagen fibrils insert into the bone matrix, is a common location of tendon injury. Commonly these injuries arise from overuse, from intrinsic tendon degeneration (tendinopathy) or from traumatic injuries.

Tendon injury leads to well characterized cellular and tissue changes that together result in altered biomechanical properties of the tendon. E.g., Arya and Kulig, J. Appl. Physiol. 108:670-675 (2010). Injury from overuse, intrinsic degeneration or from trauma may be manifest as a tendon tear. Tears are categorized by severity, from first degree minimal tears, to second degree moderate to severe tears, and finally third degree complete tears. They are also classified in other ways, such as partial or complete, in different anatomical areas of the body, such as the rotator cuff, Achilles tendon, quadriceps tendon, biceps tendon, and others.

Tears generally require surgical intervention. In some aspects, the present disclosure provides methods to produce compositions useful for repairing tendon injuries, including tears.

Furthermore, compositions of the disclosure induce tissue regeneration accelerating tendon regrowth, tendon healing, or reconstitution of the native tendon insertion into bone. The methods of the disclosure preserve endogenous growth factors present in the extracellular matrix and provide compositions for tendon regeneration, healing, and/or repair.

### SUMMARY

In an aspect, the disclosure provides a method of producing a composition comprising matrix metalloproteinase (MMP) and/or collagenase digested tendon tissue, an antimicrobial agent, and a sterile aqueous carrier solution.

In another aspect, the disclosure provides decellularized tendon matrix (DTM) composition wherein the DTM composition is prepared by a process comprising the steps of: (i) mincing a tendon tissue specimen; (ii) decellularizing the minced tendon tissue specimen; (iii) milling; (iv) digesting; (v) stopping and neutralizing; (vi) washing; and, (vii) lyophilizing.

In an aspect, the disclosure provides methods for preparing decellularized tendon matrix that preserves growth factors.

In some embodiments, the disclosure provide a decellularized tendon matrix (DTM) composition comprising matrix metalloproteinase (MMP) digested tendon tissue. In some embodiments, the disclosure provide a decellularized tendon matrix (DTM) composition comprising collagenase digested tendon tissue. In some embodiments, the composition comprises a collagen digestate. In some embodiments, the composition further comprises an antimicrobial agent. In some embodiments, the composition further comprises a sterile aqueous carrier solution. In some embodiments, the decellularized tendon matrix (DTM) is protein rich retains at least 50% of the growth factors present in the minced tendon tissue. In some embodiments, the composition is moldable. In some embodiments, the composition is able to substantially adhering to an anatomical topography.

In some embodiments, the disclosure also provides a method of making a decellularized tendon matrix (DTM) composition, the method comprising one or more steps selected from mincing a tendon tissue specimen; decellularizing the minced tendon tissue specimen; milling; digesting; stopping and neutralizing; washing; and lyophilizing. In some embodiments, the method comprises digesting with a matrix metalloproteinase (MMP) selected from the group consisting of MMP-2, MMP-9, MMP-14, or combinations thereof. In some embodiments, the method comprises digesting with a collagenase described herein. In some embodiments, the method comprises decellularizing with a DNase described herein.

The disclosure also provides a decellularized tendon matrix (DTM) composition wherein the DTM composition is prepared by a process comprising one or more steps selected from: mincing a tendon tissue specimen; decellularizing the minced tendon tissue specimen; digesting; and lyophilizing. In some embodiments, the disclosure provides a decellularized tendon matrix (DTM) composition wherein the DTM composition is prepared by a process comprising one or more steps selected from: mincing a tendon tissue specimen; decellularizing the minced tendon tissue specimen; milling; digesting; stopping; neutralizing; washing; and lyophilizing. In some embodiments, the decellularizing step comprises exposing the minced tendon tissue specimen to a solution comprising one or more components selected from a chaotropic salt, a non-ionic detergent, a zwitterionic detergent, a cationic detergent, an anionic detergent, or combinations thereof. In some embodiments, the decellularizing step comprises exposing the minced tendon tissue specimen to a DNase, an RNase, or a combination thereof. In some embodiments, the decellularizing step comprises exposing the minced tendon tissue specimen to a DNase. In some embodiments, the digesting step comprises digesting with a solution comprising a matrix metalloproteinase (MMP). In some embodiments, the matrix metalloproteinase (MMP) is selected from MMP-2, MMP-9, MMP-14, or combinations thereof. In some embodiments, the stopping and/or neutralizing step comprises stopping and/or neutralizing with a solution comprising one or more protease inhibitors selected from TAPI-0, TAPI-1, TAPI-2, marimastat, phosphoramidon, luteolin, PMSF, pepstatin A, leupeptin, E-64, sodium orthovanadate, or combinations thereof.

The disclosure also provides a method of stimulating tendon regeneration, the method comprising one or more steps selected from: resuspending a DTM composition described herein in a pharmaceutically acceptable carrier; and applying the resuspended DTM composition to a tendon site in need of stimulating tendon regeneration. In some embodiments, the resuspended DTM composition is moldable. In some embodiments, the resuspended DTM composition has a putty consistency. In some embodiments, the resuspended DTM composition is a gel. In some embodiments, the resuspended DTM composition is a paste. In some embodiments, the resuspended DTM composition is thixotropic. In some embodiments, the resuspended DTM composition is viscoelastic. In some embodiments, the resuspended DTM composition is injectable. In some embodiments, the resuspended DTM composition is spreadable.

The disclosure also provides a decellularized tendon matrix (DTM) hydrogel, comprising a resuspended DTM composition described herein, and one or more of 1-ethyl-3-[3-dimethylaminopropyl]carbodiimide (EDC) and PEG-N-hydroxysuccinimide (NHS) ester. In some embodiments, the hydrogel is moldable. In some embodiments, the hydrogel has a putty consistency. In some embodiments, the hydrogel is a paste. In some embodiments, the hydrogel is thixotropic. In some embodiments, the hydrogel is viscoelastic. In some embodiments, the hydrogel is injectable. In some embodiments, the hydrogel is spreadable.

The disclosure also provides a soft-cast decellularized tendon matrix (DTM) object, wherein the soft-cast object is prepared by a process comprising one or more steps of: resuspending a decellularized tendon matrix (DTM) composition described herein in a physiological buffer; mixing the DTM composition with PEG-N-hydroxysuccinimide (NHS) ester to produce a soft hydrogel; transferring the soft hydrogel to a three dimensional mold; curing the polymerization reaction; and inactivating the polymerization reaction.

The disclosure also provides a decellularized tendon matrix (DTM) hydrogel comprising a resuspended DTM composition described herein, further comprising 1-ethyl-3-[3-dimethylaminopropyl]carbodiimide (EDC) and a water-soluble coupling agent selected from N-hydroxysuccinimide (NHS) or a N-hydroxysulfosuccinimide (sulfoNHS) in conjunction with the (EDC) coupling agent.

The disclosure also provides a method of treating a tendon tear and/or stimulating tendon regeneration in a subject, the method comprising one or more of: obtaining a decellularized tendon matrix (DTM) composition comprising matrix metalloproteinase (MMP) or collagenase digested tendon tissue; resuspending the DTM composition in a pharmaceutically acceptable carrier; and applying the resuspended DTM composition to a tendon site in need of stimulating tendon regeneration.

the disclosure also provides a decellularized tendon matrix produced from a native tendon, the decellularized tendon matrix comprising greater than 90% by weight of TGF-β in the native tendon. In some embodiments, the decellularized tendon matrix comprises greater than 95% by weight of TGF-β in the native tendon. In some embodiments, the decellularized tendon matrix comprises greater than 99% by weight of TGF-β in the native tendon. In some embodiments, the decellularized tendon matrix of any one of claims 18-20, comprises less than 5% by weight of cellular material in the native tendon. In some embodiments, the decellularized tendon matrix described herein comprises less than 2% by weight of cellular material in the native tendon. In some embodiments, the decellularized tendon matrix described herein comprises less than 1% by weight of cellular material in the native tendon. In some embodiments, the decellularized tendon matrix described herein comprises less than 0.1% by weight of cellular material in the native tendon. In some embodiments, the decellularized tendon matrix described herein comprises is substantially free of TGF-β producing cells. In some embodiments, the decellularized tendon matrix described herein comprises less than 5% by weight of DNA in the native tendon. In some embodiments, the decellularized tendon matrix described herein comprises less than 2% by weight of DNA in the native tendon. In some embodiments, the decellularized tendon matrix described herein comprises less than 1% by weight of DNA in the native tendon. In some embodiments, the decellularized tendon matrix described herein comprises less than 0.1% by weight of DNA in the native tendon. In some embodiments, the decellularized tendon matrix described herein is substantially free of DNA.

The disclosure also provides a method of producing a decellularized tendon matrix (DTM) composition from a tendon, the method comprising one or more of: decellularizing the tendon thereby producing a decellularized tendon; contacting the decellularized tendon with an enzymatic solution comprising a matrix metalloproteinase (MMP) to produce a digested, decellularized tendon; lyophilizing the digested, decellularized tendon to produce a lyophilized tendon; and reconstituting the lyophilized tendon to produce a decellularized tendon matrix. In some embodiments, the method comprises contacting the tendon with a DNase solution. In some embodiments, the DNase solution comprises about 10 to about 100 Units of DNase per milliliter of solvent, about 25 to about 75 Units of DNase per milliliter of solvent, about 40 to about 60 Units of DNase per milliliter of solvent, about 40 to about 60 Units of DNase per milliliter of solvent, or about 50 Units of DNase per milliliter of solvent. In some embodiments, the decellularizing comprises contacting the tendon with between about 4 milliliters and about 50 milliliters of the DNase solution per 1 gram of tendon. In some embodiments, the decellularizing comprises contacting the tendon with between about 5 milliliters and about 10 milliliters of the DNase solution per 1 gram of tendon. In some embodiments, the decellularizing comprises contacting the tendon with between about 10 milliliters and about 50 milliliters of the DNase solution per 1 gram of tendon. In some embodiments, the contacting occurs for a period of about 1 hour, and optionally occurs on a shaker. In some embodiments, the decellularizing further comprises washing the tendon with phosphate buffered saline. In some embodiments, the decellularizing further comprises filtering the tendon. In some embodiments, the lyophilizing comprises freezing the digested, decellularized tendon at minus 80° C. for at least about 30 minutes. In some embodiments, the method further comprises filtering through a 70 micrometer strainer using centrifugation at between about 1500 G to about 2500 G for between about 1 minute and about 15 minutes. In some embodiments, the MMP comprises collagenase. In some embodiments, the collagenase is selected from the group consisting of Collagenase Type I, Collagenase Type III, and a combination thereof. In some embodiments, the concentration of the Collagenase Type I in the enzymatic solution is about 2 milligrams per milliliter. In some embodiments, the concentration of the Collagenase Type III in the enzymatic solution is about 1 milligram per milliliter. In some embodiments, the decellularized tendon is contacted with between about 10 milliliters and about 50 milliliters of the enzymatic solution per 1 gram of tendon. In some embodiments, the decellularized tendon is contacted with between about 5 milliliters and about 10 milliliters of the enzymatic solution per 1 gram of tendon. In some embodiments, the decellularized tendon is contacted with the enzymatic solution for about 24 hours. In some embodiments, the decellularized tendon is contacted with the enzymatic solution for about 12 hours. In some embodiments, the decellularized tendon is contacted with the enzymatic solution for about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17, about 18 hours, about 19 hours, about 20 hours, about 21 hours, about 22 hours, about 23 hours, or about 24 hours. In some embodiments, the decellularized tendon is contacted with the enzymatic solution at about 37° C. In some embodiments, the reconstituting comprises mixing between about 2 microliters and about 5 microliters of solvent with about 1 milligram of lyophilized tendon.

### BRIEF DESCRIPTION OF THE DRAWINGS

FIGS. 1A-B illustrate native tendon characterization of DNA content (FIG. 1A) and protein content (FIG. 1B) in tendon prior to processing native patella and Achilles tendons. Measurements depict tendons from a total of 6 donors.

FIGS. 2A-B illustrate native TGF-β concentrations based on tendon type and location. TGF-β3 concentration (FIG. 2A) and TGF-β1 concentration (FIG. 2B) found in native tendon samples (prior to processing) are shown.

FIG. 3 illustrates a comparison of decellularization using DNase and detergents. DNA content in both patella and Achilles tendons is measured in native tendon, tendon treated with DNase 50 U for 1 hour, tendon treated with DNase 50 U for 2 hours, and tendons treated with traditional decellularization methods using SDS or EDTA.

FIG. 4 illustrates total protein of tendons using various enzymatic reagents to digest the tendon samples, including C-1 Collagenase I, C-3 Collagenase III, both C-1 Collagenase I and C-3 Collagenase III, and pepsin.

FIG. 5 illustrates TGF-β concentrations before and after processing tendon into decellularized tendon matrix. Native tendon is measured by averaging all proximal, mid-substance and distal portions of both patella and Achilles tendons.

FIGS. 6A-F illustrate that decellularized tendon matrix processing facilitates an elastic characteristic which has the capacity to stretch (FIG. 6A) from an unstretched conformation (FIG. 6B) without being pulled apart. DTM is storage stable as a sterile lyophilized powder and can be reconstituted into a putty or an injectable solution. This image shows the DTM putty which can be formed by resuspending the lyophilized DTM with 3-5 ul/mg. This putty is moldable/stretchable for surgical application to the desired region of repair. FIG. 6C: Lyophilized. FIG. 6D: Reconstituted. FIG. 6E: Reconstituted, 48 hours. FIG. 6F: Oscillation stress sweep of human DTM with concentrations of 0, 1, 3, 5, and 7 g of lyophilized DTM to 1 mL of PBS. Complex modulus is expressed on the y-axis (Pa) and oscillation stress is expressed on the x-axis (Pa) resulting in all reconstitution concentrations tested maintain a well-formed elastic structure under low stress conditions.

FIGS. 7A-C illustrate that DNAse treatment effectively decellularizes tendon tissue. Tendon was decellularized using various concentrations of DNAse (10 U, 50 U, and 100 U) over 1 hour. 1×PBS was used as a control for no decellularization. DNA concentration was determined using DNEasy kits (Qiagen). This data shows that as little as 50 U of DNAse is effective in decellularizing tissue.

FIG. 8 illustrates that DNAse treatment is as effective as standard detergent methods at decellularizing tendon. DNAse at 50 U was compared to traditional detergents, 1% SDS and 0.1% EDTA. DNAse 50 U was tested at 0.5 hours, 1 hours, and 2 hours, while standard SDS and EDTA protocol calls for a 24-hour decellularization. DNA concentration was determined using DNEasy kits (Qiagen, n=3). All values were normalized to no decellularization. Tukey's HSD multiple comparison post-hoc testing shows no significant difference between the different times of DNAse treatment or decellularization by DNAse versus SDS and EDTA.

FIGS. 9A-H illustrate that Achilles tendon matrix has more protein content than patella tendon. The Achilles and Patellar tendons were divided into ⅓ sections consisting of the proximal, midcenter/middle, and distal ends of the tendon. (A-D) Total protein of the native tendons was measured using a BCA protein quantification kit (Thermo Scientific). (E-H) TGF-β was measured using a TGF-β magnetic bead panel Milliplex kit (Millipore Sigma, #TGFBMAG-64K-03). ANOVA shows no statistically significant differences between the regions of the tendons and therefore the entirety of the tendon can used through processing. When comparing the two different tendons (D) total protein is not different (P=0.93), but (H) TGF-β is statistically higher in Achilles than Patellar tendon (P=0.0045).

FIG. 10A: Collagenase activity was measured to confirm successful removal of the added collagenase solution during enzymatic digestion. No significant differences in collagenase activity between native tendon (n=3) and enzymatically digested and filtered samples (n=10; p=0.922). Following filtration, the TGFβ profile was tested to confirm bioactivity was maintained (n=10). No statistical significance was found in (FIG. 10B) TGFβ1 and in (FIG. 10C) TGFβ2 between combined native tendons and DTM. However, statistical significance was found in (FIG. 10D) TGFβ3 between the combined native tendons and DTM (p=0.0189). FIG. 10E illustrates that filtering effectively eliminates collagenase activity. Decellularized tendon was treated with collagenase to improve form-factor of DTM. 100 kDa filters were highly effective in eliminating the collagenase activity in the final product. ANOVA indicates that the groups have significant differences (F (4, 22)=18.06, p<0.0001). Importantly, there are no significant differences in collagenase activity between native and 100 kDa filtered samples.

FIG. 11 illustrates that more bioactivity is retained in DTM than standard methods for decellularizing tendon with pepsin. Tendons were digested following decellularization, using a solution containing Collagenase Type 1 (92.5 g tendon/g Collagenase 1) and 3 (185 g tendon/1 g Col 3), or using Pepsin given previous published methodologies (Farnebo et. al 2014, PMID: 24341855). ANOVA indicated significant differences between groups, F (3,11)=5.056, p=0.0193. Tukey's HSD post hoc shows pepsin has significantly less TGF-β (P=0.0249).

FIGS. 12A-G illustrate differences in proliferation of cells plated on different surfaces. Tissue culture plates were left untreated (control, “TC treated”), coated with collagen or with the DTM. Primary tenocytes (ZenBio #TEN-F) were plated at 20,000 cells/well and cell viability quantified using the Presto Blue (Thermo Fisher) at (A) 48 hours after plating, (B) 7 days after plating, generating significantly different growth rates (C), or (D) 48 hours after plating, (E) 7 days after plating, generating significantly different growth rates (F). (ANOVA=F (3,26)=10.6, p<0.0001). (G) shows the statistical analysis for the corresponding data in (D)-(F).

FIGS. 13A-J illustrate primary tenocytes and ADSCs were plated at 20,000 cells/well and cell viability quantified at (FIG. 13A, 13B) 48 hours and (FIG. 13C, 13D) 7 days after plating, generating significantly different growth rates (n=4-9/group). (FIG. 13E-13J) Collagen coating, tissue-culture treated and DTM coated plates were seeded with tenocytes and still images from live cell imaging were taken at 0 (E-G) and 48 hours (FIG. 13H-13J), showing the variance in tenocyte cell morphology on the DTM compared (FIG. 13G/13J) to the standard tissue culture (E/H), and collagen coated plate (F/I). Scale bars=200 μm; also illustrated are differences in morphology and/or proliferation of cells plated on different surfaces. Tissue culture plates were left untreated (control, “TC treated”), coated with collagen or with the DTM. Primary tenocytes (ZenBio #TEN-F) were plated at 20,000 cells/well. Live cell images were taken by time laps video over 3 days showing significantly different cell morphology and proliferation rates between the different surface treatments. Still images from the live cell imaging were taken at 48 hours and show that tenocytes more rapidly adhere, proliferate and with increased focal adhesion and a more native like cell morphology on the DTM compared (FIG. 13G, 13J) to the standard tissue culture (FIG. 13E, 13H) or collagen coated plate (FIG. 13F, 13I).

FIGS. 14A-C illustrates images of surgical application of DTM. DTM can be formed into a putty or an injectable solution. In this case the putty was placed upon the greater tuberosity and the supraspinatus surgically attached to secure the DTM. (FIG. 14A) Initial rotator cuff tear of the supraspinatus; (FIG. 14B) DTM placed under the supraspinatus 6 weeks after the initial tear; (FIG. 14C) repair of the initial tear, suture-anchoring the supraspinatus to the greater tuberosity; (FIG. 14D) HBQ on the repair only (FIG. 14E) and the DTM with repair, displaying the bone (red) and cartilage (blue) tissue formation in the osteotendinous junction; (FIG. 14F) H&E on the mid-substance of the tendon for repair only (FIG. 14G) and the DTM with repair showing increased cellularity of the tendon with DTM treatment. Scale bars=100 μm

FIG. 15 illustrates the normalized TGFb content across four samples from four different donors, over the two processing steps. For each respective donor, the first column represents the amount of TGFb in the native tendon, the second column represents the amount of TGFb in the decellularized tendon, and the third column represents the amount of TGFb in the digested tendon.

FIGS. 16A-16L illustrate that Native Patella and Achilles Tendon have similar bioactive potential. The Achilles and Patellar tendons were divided into ⅓ sections consisting of the proximal, midcenter/middle, and distal ends of the tendon. Total protein of the native tendons was measured using a BCA protein quantification kit (Thermo Scientific). TGF-β was measured using a TGF-β magnetic bead panel (Millipore Sigma, #TGFBMAG-64K-03). (FIG. 16A) Total protein content between native Achilles and patella tendons was analyzed and no significant difference was found between the tendon types (n=10). (FIG. 16B-C) The Achilles and patella tendons had no significance in total protein content between proximal, mid, and distal regions. (FIG. 16J) shows the statistical analysis for the corresponding data in (FIG. 16A)-(FIG. 16C). (FIG. 16D-16F) Achilles and patella data were combined to analyze variation in TGFβ content between the proximal, mid-center and distal regions (n=10). No statistical difference was detected in TGFβ content between regions. (FIG. 16K) shows the statistical analysis for the corresponding data in (FIG. 16D)-(FIG. 16F). (FIG. 16G) TGFβ1 (p=0.0137) (FIG. 16H) and TGFβ2 (p=0.0283) were significantly lower in Achilles compared to patellar tendon, but no difference was found in (FIG. 16I) TGFβ3 between the tendons. (FIG. 16L) shows the statistical analysis for the corresponding data in (FIG. 16G)-(FIG. 16I).

FIGS. 17A-17F illustrate that DNAse treatment effectively decellularizes tendon tissue. Patella tendon and Achilles Tendon were decellularized using various concentrations of DNAse (10 U, 50 U, and 100 U) over 1 hour (FIG. 17A) 1×PBS was used as a control for no decellularization. This data shows that as little as 50 U of DNAse is effective in decellularizing tissue. (FIG. 17B) DNAse at 50 U was compared to traditional detergents, 1% SDS and 0.1% EDTA. DNAse 50 U was tested at 0.5 hours, 1 hours, and 2 hours, while standard SDS and EDTA protocol calls for a 24-hour decellularization. All values were normalized to no decellularization. Tukey's HSD multiple comparison post-hoc testing shows no significant difference between the different times of DNAse treatment or decellularization by DNAse versus SDS and EDTA. Histological sections of (FIG. 17C) Native tendon or (D) DTM decellularization stained with DAPI to identify cell nuclei, scale bar=100 μm; DAPI of native tendon (4× magnification). (FIG. 17D) DAPI of decellularized tendon by 50 U DNAse for 2 hours (4× magnification). (FIG. 17E) shows the statistical analysis for the corresponding data in (FIG. 17A)-(FIG. 17D). (FIG. 17F) DNA content was measured following decellularization using the DTM processing technique or standard detergents methods 1% SDS or 0.1% EDTA and normalized to non-treated PBS control samples (n=2).

FIG. 18 illustrates the collagenase activity of DTM with and without filtration.

FIGS. 19A-19D illustrate that enzymatic digestion maintains TGFβ bioactivity. (FIG. 19E) shows the statistical analysis for the corresponding data in (FIG. 19A)-(FIG. 19D).

FIGS. 20A-20B illustrate no statistically significant differences found between males and females. (FIG. 20A) Illustrates donor pool demographics for total protein content, and (FIG. 20B) illustrates total protein content in males and females and between Achilles and patellar tendons. No statistical difference was found in total protein content between male and female Achilles and patellar tendons.

FIGS. 21A-21C illustrate the concentration of (FIG. 21A) TGF-β1, (FIG. 21B) TGF-β2, and (FIG. 21C) TGF-β3 in male and female tendon. (FIG. 20D) shows the statistical analysis for the corresponding data in FIGS. 20A-20B and FIGS. 21A-21C.

FIG. 22 illustrates mean force to failure in rabbit tendons in which a supraspinatus tendon tear was repaired with and without the use of DTM.

FIG. 23 illustrates a schematic diagram of tendon dissection.

FIG. 24. To determine the gene expression of tendon stem cell markers qRT-PCR was done on both ADSCs (n=3) and tenocytes (n=3) to look at Scleraxis and Tenomodulin expression, with both cell types expressing an upregulation of Tnmd at day 2 and normalized again by day 7 (FIG. 24A-24B), and a normalized level of Scx in both cell types at day 2 with a significant upregulation in both cell types seen at day 7 (FIG. 24C-24D).

FIG. 25 illustrates the Complex Modulus (Pa) v Oscillation Stress (Pa).

FIG. 26 illustrates Complex Modulus (Pa) v Oscillation Stress (Pa)—All Samples.

FIG. 27 illustrates Complex Modulus (Pa) v Oscillation Stress (Pa)—Rabbit sample only.

FIG. 28 illustrates Phase Angle (°) v Oscillation Stress (Pa).

FIG. 29 illustrates Storage Modulus (Pa) and Loss Modulus (Pa) v Angular Frequency (rad/s)—Sample A.

FIG. 30 illustrates Storage Modulus (Pa) and Loss Modulus (Pa) v Angular Frequency (rad/s)—Sample B.

FIG. 31 illustrates Storage Modulus (Pa) and Loss Modulus (Pa) v Angular Frequency (rad/s)—Sample C.

FIG. 32 illustrates Storage Modulus (Pa) and Loss Modulus (Pa) v Angular Frequency (rad/s)—Sample D.

FIG. 33 illustrates Storage Modulus (Pa) and Loss Modulus (Pa) v Angular Frequency (rad/s)—Sample E.

### DETAILED DESCRIPTION

Rotator cuff tears are a common soft tissue injury that can significantly decrease function of the shoulder and cause severe pain. In many cases surgical repair of the rotator cuff is performed to address pain and improve joint function. Despite progress in surgical technique, rotator cuff repairs (RCR) do not always heal as quickly or as well as expected. This is especially true for repair of massive and/or chronic tears, which account for up to 40% of all rotator cuff tears with failure rates exceeding 50% in some studies. The majority of failures occur at the bone-tendon interface as a result of poor healing capacity of the tendon and failure to regenerate the native histological anatomy of the enthesis. While allografts are commercially available for use in RCR clinical use is limited as they do not stimulate tissue regeneration and are associated with a structural failure of up to 67% in re-tear cases. Novel tissue engineering strategies are being developed with great promise, but most involve addition of cells or growth factors that make clinical translation complicated. As a result, there is a significant unmet clinical need for easily translatable surgical augmentation approaches that can improve healing in RCR. In this study, it is described the development of a decellularized tendon matrix (DTM) putty that preserves TGFβ bioactivity by implementing a novel processing technique. In vitro, DTM promotes proliferation of tenocytes, and adipose-derived stem cells with an increase in expression of tendon-specific transcription factors Scleraxis and Tenomodulin. When placed in a rabbit model of a chronic rotator cuff tear, DTM improves histological tissue repair by increasing cellularity of the repair and promoting calcification at the bone-tendon interface more similar to the normal fibrocartilaginous enthesis. Taken together, these data indicate the engineered DTM putty retains a pro-regenerative bioactivity that presents a promising translational strategy for improving healing at the tendon-bone interface.

Tendon and ligament injuries affect approximately 17 million Americans each year and are the second leading cause of musculoskeletal injuries. It has been found that certain tendons are prone to higher rates of injury, including rotator cuff, forearm extensor, Achilles, and patellar tendons. In 2016, there were over 460,000 rotator cuff surgeries performed in the United States and it was the second most common orthopaedic soft tissue repair procedure performed. Unfortunately, despite significant clinical progress to improve surgical technique and rehabilitation protocols, structural outcomes of rotator cuff repair (RCR) remain statistically poor. Re-tear rates as high as 40% have been noted following standard rotator cuff repairs with failures increasing to 73-94% in massive tears. In addition to the higher re-tear rates with increased tear size, there is also an increase incidence of repair failures with increasing patient age.

Achieving effective repair at the tendon-bone junction is critical in RCR as this is where the vast majority of post-operative failures occur. Insertion of the tendon into the bone at the rotator cuff occurs through a fibrocartilaginous enthesis where there is a gradual histological transition from tendon to fibrocartilage to calcified fibrocartilage to bone. A normal enthesis does not regenerate following RCR, rather a mechanically weak fibrovascular scar, distinctly lacking the zone of calcified cartilage, is observed at the tendon-bone junction. A number of critical factors likely contribute to the poor regenerative capacity of the tendon, including a paucity of appropriate tendon progenitor cells, the low vascularity of the fibrocartilaginous enthesis, and limited bioavailability of growth factors that can promote regeneration. Given the poor clinical results of RCR, there is currently a major challenge in the field of orthopedic medicine to stimulate biological healing and reconstruct the enthesis structure in order to regenerate a stronger tendon.

Interestingly, while adult tendon tissues heal poorly, neonatal tendons can successfully regenerate following injury in a process termed “scarless healing”. Lineage tracing identified Scleraxis (Scx) positive tendon progenitor cells as the key cellular population enabling neonatal tendon regeneration. In the adult tissue, these Scx-lineage tenocytes are not recruited to the injury, and there is very minimal cellular proliferation 3 days post-injury. Regulation of tenocyte recruitment and the subsequent functional tendon regeneration appears to be directly regulated by Transforming Growth Factor-β (TGFβ) signaling in the neonatal tissue.

A decellularized tendon matrix (DTM) putty is developed utilizing a novel enzymatic processing technique to preserve native TGFβ bioactivity and promote cell proliferation. Decellularized matrices have been utilized extensively in bone regeneration strategies to provide a biomimetic and bioactive substrate to support structural and biologic healing. Tendon and dermal allografts are commercially available to support RCR, but their use has been limited due to the combination of unclear effect on clinical outcomes and limited evidence of bony ingrowth from the tuberosity into the allograft. Engineered decellularized matrices provide an opportunity to improve upon allograft technology by preserving the bioactivity of the tissue and generating a clinically more useful form factor for surgical application. Without wishing to be bound by any particular theory, it is hypothesized that the novel decellularized tendon matrix putty with preserved TGFβ would enhance tendon-to-bone healing following RCR in a rabbit chronic rotator cuff tear model.

Unless defined otherwise, all technical and scientific terms used herein have the same meaning as is commonly understood by one of skill in the art to which this disclosure belongs. All patents and publications referred to herein are incorporated by reference in their entireties.

### Definitions

The terms “co-administration,” “co-administering,” “administered in combination with,” “administering in combination with,” “simultaneous,” and “concurrent,” as used herein, encompass administration of two or more active pharmaceutical ingredients. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

“Treatment”, “treating”, “palliating” and “ameliorating”, as used herein, are used interchangeably. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, “Donor” refers to a mammalian source for tendon connective tissue. The donor may be human or other animal source, including cadaveric tendon tissue. “Allogenic” donor tissue is donor tissue from a non-genetically identical member of the same species, for example, harvested from one human subject, then administering the resulting composition to a different human subject. Tendon connective tissue can be harvested from a donor that is of another species for use in the methods herein to produce decellularized tendon matrix compositions; such compositions are “xenographic” decellularized tendon matrix compositions. Preferred xenograph sources are pig, horse, cow, sheep, dog, and rodent. No matter the source, xenograph tendon tissue may be fresh or fresh-frozen tissue from a cadaveric donor. Preferred allograft sources are Achilles and patellar tendons. These tendons are readily available and are relatively large in size. They are also used widely in autograft and allograft application for the reconstruction of torn or damaged ligaments and tendons.

“Decellularization” as used herein refers to the general (at least 80%), nearly complete (at least 95%), or essentially complete (at least 99%) removal of cellular components of tendon connective tissue.

As used herein, “matrix metalloproteinases” refers to proteins of the matrix metalloproteinase (MMP) family. Matrix metalloproteinases (MMPs) comprise a large family of zinc-dependent endoproteinases, collectively capable of degrading all extracellular matrix (ECM) components. The term encompasses both the apo- and activated forms of each MMP family member. The term encompasses MMP-2, MMP-9, MMP-14, homologs, derivatives, and fragments thereof. Fanjul-Fernandez et al. summarize the mammalian MMP family in a review article, Biochim. Biophy. Acta 1803:3-19 (2010).

Various growth factors are known to the art, including: IGF-1 (Insulin-like growth factor 1, or somatomedin C), TGF-β (transforming growth factor beta), PDGF (Platelet-derived growth factor), VEGF (Vascular endothelial growth factor (VEGF), also known as vascular permeability factor (VPF)), bFGF (basic fibroblast growth factor, or fibroblast growth factor 2 (FGF2)), GDF-5 (Growth differentiation factor 5), GDF-6 (Growth differentiation factor 6), GDF-7 (Growth differentiation factor 7), HGF (hepatocyte growth factor or scatter factor). Without being bound by theory, the above non-limiting list of growth factors are known to the art to be found in the extracellular matrix of tendons.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the DTM ingredient, its use in the therapeutic compositions of the disclosure is contemplated. Additional active pharmaceutical ingredients, such as other drugs, can also be incorporated into the described compositions and methods.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

The terms “sequence identity,” “percent identity,” and “sequence percent identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, Calif.) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

For the avoidance of doubt, it is intended that particular features (for example integers, characteristics, values, uses, diseases, formulae, compounds or groups) described in conjunction with a particular aspect, embodiment or example of the disclosure are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any disclosed embodiments. The disclosure extends to any novel one, or novel combination, of the features disclosed in this specification (including any accompanying claims, abstract and drawings), or to any novel one, or any novel combination, of the steps of any method or process so disclosed.

### Methods of Making Decellularized Tendon Matrix

One goal of the embodiments of the present disclosure is to produce a DTM that preserves growth factors, specifically TGF-β, in the matrix by developing a gentle and specific decellularization and digestion protocol. Traditionally detergents are harsh and can remove or denature proteins as well as the cellular material.

Typical digestion techniques for decellularized matrices use general proteinases, most commonly pepsin, which indiscriminately cleaves all proteins into small polypeptides. In this application enzymes specific for cleavage of collagen are used in order to break down the tendon into smaller parts that can subsequently form self-assembling peptide. Collagens, predominantly collagen type I, forms the structural backbone of tendon. By specifically cleaving the collagen we digest the tendon, but preserve the bioactivity of growth factors attached.

Collagenases are endopeptidases that digest the triple-helical native collagen fibrils commonly found in tendon. Collagenase cleaves the bond between a neutral amino acid (X) and glycine in the sequence Pro-X-Gly-Pro, which is found with high frequency in collagen. Bacterial collagenase, such as that made by Clostridium histolyticum, can attack almost all collagen types and degrades both water-insoluble native collagens and water-soluble denatured ones. Clostridial collagenases' ability to digest native, triple-helical types I, II, and III collagens through multiple scissions in the triple helix is a primary distinguishing factor. Clostridium collagenases represent unusually large metalloproteases, a family of proteases that shares a zinc-containing motif at the center of the active site (Gonzales and Robert-Baudouy 1996).

Matrix metalloproteinases (MMP) also have the ability to cleave collagen fibers in very specific sequences. Interstitial collagen types I, II and III are highly resistant to proteolytic attack, due to their triple helical structure, but can be cleaved by MMP collagenases at a specific sites. MMP-2 and -9 are closely related at the structural level and have demonstrated collagenase activity on collagen types I and III, generating the classic ¾ and ¼ fragments. MMP-1, MMP-8, MMP-13, the MT-MMPs also have some limited collagenase activity.

In an aspect, the disclosure provides a method of producing a composition comprising matrix metalloproteinase (MMP) digested tendon tissue, an antimicrobial agent, and a sterile aqueous carrier solution. In some embodiments, the matrix metalloproteinase (MMP) is selected from the group consisting of MMP-2, MMP-9, MMP-14, or combinations thereof. In an aspect, the MMP is engineered to be constitutively active. A person having skill in the art will appreciate that other MMPs can be used. Collagenases, the gelatinases, the stromelysins, and the membrane-type MMPs (MT-MMPs) can be used. In certain embodiments, collagenase can be used to decellularized a tendon and/or digest a decellularized tendon. As described herein, collagenases are capable of degrading triple-helical fibrillar collagens into distinctive ¾ and ¼ fragments. These collagens are the major components of bone, cartilage and dentin. Collagenases include Collagenase Type 1, Collagenase Type 2, Collagenase Type 3, Collagenase Type 8, Collagenase Type 13, Collagenase Type 14, and Collagenase Type 18. Non-limiting examples of one or more MMPs that can be used include MMP1 (Interstitial collagenase, CLG, CLGN), MMP2 (Gelatinase-A, 72 kDa gelatinase), MMP3 (Stromelysin 1, CHDS6, MMP-3, SL-1, STMY, STMY1, STR1), MMPI (Matrilysin, PUMP 1, MMP-7, MPSL1, PUMP-1), MMP8 (Neutrophil collagenase, CLG1, HNC, MMP-8, PMNL-CL), MMP9 (Gelatinase-B, 92 kDa gelatinase, CLG4B, GELB, MANDP2, MMP-9), MMP10 (Stromelysin 2, SL-2, STMY2), MMP11 (Stromelysin 3, SL-3, ST3, STMY3), MMP12 (Macrophage metalloelastase, HME, ME, MME, MMP-12), MMP13 (Collagenase 3, CLG3, MANDP1, MMP-13), MMP14 (MT1-MMP, MMP-14, MMP-X1, MT-MMP, MT-MMP 1, MT1-MMP, MT1MMP, MTMMP1, WNCHRS), MMP15 (MT2-MMP, MT2-MMP, MTMMP2, SMCP-2, MMP-15, MT2MMP), MMP16 (MT3-MMP, C8orf57, MMP-X2, MT-MMP2, MT-MMP3, MT3-MMP), MMP17 (MT4-MMP, MT4-MMP, MMP-17, MT4MMP, MTMMP4), MMP18 (Collagenase 4, xcol4, xenopus collagenase), MMP19 (RASI-1, occasionally referred to as stromelysin-4, MMP18, RASI-1, CODA), MMP20 (Enamelysin, AI2A2, MMP-20), MMP21 (X-MMP, MMP-21, HTX7), MMP23A (CA-MMP), MMP23B (MIFR, MIFR-1, MMP22), MMP24 (MT5-MMP, MMP-24, MMP25, MT-MMP 5, MT-MMP5, MT5-MMP, MT5MMP, MTMMP5), MMP25 (MT6-MMP, MMP-25, MMP20, MMP20A, MMPL1, MT-MMP 6, MT-MMP6, MT6-MMP, MT6MMP, MTMMP6), MMP26 (Matrilysin-2, endometase), MMP27 (MMP-22, C-MMP, MMP-27), and MMP28 (Epilysin, EPILYSIN, MM28, MMP-25, MMP-28).

The concentration of collagenase used to enzymatically digest decellularized tendon can vary depending on the specific collagenase used. In certain embodiments Collagenase Type 1 can be used to enzymatically digest decellularized tendon. In certain embodiments Collagenase Type 3 can be used to enzymatically digest decellularized tendon. The concentration of collagenase used to enzymatically digest decellularized tendon can be about 0.1 milligram (mg)/milliliter (mL), about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2.0 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, about 3.0 mg/mL, about 3.1 mg/mL, about 3.2 mg/mL, about 3.3 mg/mL, about 3.4 mg/mL, about 3.5 mg/mL, about 3.6 mg/mL, about 3.7 mg/mL, about 3.8 mg/mL, about 3.9 mg/mL, about 4.0 mg/mL, about 5.0 mg/mL, about 6.0 mg/mL, about 7.0 mg/mL, about 8.0 mg/mL, about 9.0 mg/mL, or about 10.0 mg/mL. In certain embodiments, the concentration of collagenase used to enzymatically digest decellularized tendon is about 1.0 mg/mL. In other embodiments, the concentration of collagenase used to enzymatically digest decellularized tendon is about 2.0 mg/mL.

The antimicrobial agent is a suitable agent for use in a parenteral formulation, for example, an alkyl alcohol or an aryl alcohol, such as benzyl alcohol, chlorbutanol, or 2-ethoxyethanol. Amino aryl acid esters are also suitable, for example, methyl, ethyl, propyl, or butyl parabens and combinations thereof. Alkyl acids and aryl acids may also be suitable, for example, benzoic acid or sorbic acid; biguanides, for example, chlorhexidine or phenols, for example phenol or 3-cresol. In some embodiments, combinations of chemically compatible antimicrobial agents are used.

In an aspect, the present disclosure provides a decellularized tendon matrix (DTM) composition wherein the DTM composition is prepared by a process comprising the steps of: (i) mincing a tendon tissue specimen; (ii) decellularizing the minced tendon tissue specimen; (iii) digesting; and, (iv) lyophilizing.

In another aspect, the disclosure provides decellularized tendon matrix (DTM) composition wherein the DTM composition is prepared by a process comprising the steps of: (i) mincing a tendon tissue specimen; (ii) decellularizing the minced tendon tissue specimen; (iii) milling; (iv) digesting; (v) stopping and neutralizing; (vi) washing; and, (vii) lyophilizing.

In some cases, prior to decellularization, milling, digesting, lyophilizing, and/or washing the tendon matrix can be present in a portion of about 40%, about 45%, about 50%, about 55%, about 60%, about 65%, about 70%, about 75%, about 80%, about 85%, about 90%, about 95%, or about 99% by weight of the isolated tendon tissue.

In some cases, prior to decellularization, milling, digesting, lyophilizing, and/or washing, the tendon matrix can be present in a portion from about 50% to about 90%, about 50% to about 80%, about 50% to about 70%, about 50% to about 60%, about 50% to about 55%, 60% to about 90%, about 60% to about 80%, about 60% to about 70%, about 60% to about 65%, 70% to about 90%, about 70% to about 80%, about 70% to about 75%, 80% to about 90%, about 80% to about 85%, or about 85% to about 90% by weight of the isolated tendon tissue.

In some cases, prior to decellularization, milling, digesting, lyophilizing, and/or washing, the tendon matrix can be present in a portion of less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, or less than about 10% by weight of the isolated tendon tissue.

In some cases, prior to decellularization, milling, digesting, lyophilizing, and/or washing, the tendon matrix can be present in a portion of about 40%, about 45%, about 50%, about 55%, about 60%, about 65%, about 70%, about 75%, about 80%, about 85%, about 90%, about 95%, or about 99% by volume of the isolated tendon tissue.

In some cases, prior to decellularization, milling, digesting, lyophilizing, and/or washing, the tendon matrix can be present in a portion from about 50% to about 90%, about 50% to about 80%, about 50% to about 70%, about 50% to about 60%, about 50% to about 55%, 60% to about 90%, about 60% to about 80%, about 60% to about 70%, about 60% to about 65%, 70% to about 90%, about 70% to about 80%, about 70% to about 75%, 80% to about 90%, about 80% to about 85%, or about 85% to about 90% by volume of the isolated tendon tissue.

In some cases, prior to decellularization, milling, digesting, lyophilizing, and/or washing, the tendon matrix can be present in a portion of less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, or less than about 10% by volume of the isolated tendon tissue.

In some cases, after decellularization, milling, digesting, lyophilizing, and/or washing, the tendon matrix can be present in a portion of about 40%, about 45%, about 50%, about 55%, about 60%, about 65%, about 70%, about 75%, about 80%, about 85%, about 90%, about 95%, or about 99% by weight of the decellularized, milled, digested, lyophilized, and/or washed tendon tissue.

In some cases, after decellularization, milling, digesting, lyophilizing, and/or washing, the tendon matrix can be present in a portion from about 50% to about 90%, about 50% to about 80%, about 50% to about 70%, about 50% to about 60%, about 50% to about 55%, 60% to about 90%, about 60% to about 80%, about 60% to about 70%, about 60% to about 65%, 70% to about 90%, about 70% to about 80%, about 70% to about 75%, 80% to about 90%, about 80% to about 85%, or about 85% to about 90% by weight of the decellularized, milled, digested, lyophilized, and/or washed tendon tissue.

In some cases, after decellularization, milling, digesting, lyophilizing, and/or washing, the tendon matrix can be present in a portion of greater than about 99%, greater than about 95%, greater than about 90%, greater than about 85%, greater than about 80%, greater than about 75%, greater than about 70%, greater than about 65%, greater than about 60%, greater than about 55%, greater than about 50%, greater than about 45%, greater than about 40%, greater than about 35%, greater than about 30%, greater than about 25%, greater than about 20%, greater than about 15%, or greater than about 10% by weight of the decellularized, milled, digested, lyophilized, and/or washed tendon tissue.

In some cases, after decellularization, milling, digesting, lyophilizing, and/or washing, the tendon matrix can be present in a portion of about 40%, about 45%, about 50%, about 55%, about 60%, about 65%, about 70%, about 75%, about 80%, about 85%, about 90%, about 95%, or about 99% by volume of the decellularized, milled, digested, lyophilized, and/or washed tendon tissue.

In some cases, after decellularization, milling, digesting, lyophilizing, and/or washing, the tendon matrix can be present in a portion from about 50% to about 90%, about 50% to about 80%, about 50% to about 70%, about 50% to about 60%, about 50% to about 55%, 60% to about 90%, about 60% to about 80%, about 60% to about 70%, about 60% to about 65%, 70% to about 90%, about 70% to about 80%, about 70% to about 75%, 80% to about 90%, about 80% to about 85%, or about 85% to about 90% by volume of the decellularized, milled, digested, lyophilized, and/or washed tendon tissue.

In some cases, after decellularization, milling, digesting, lyophilizing, and/or washing, the tendon matrix can be present in a portion of greater than about 99%, greater than about 95%, greater than about 90%, greater than about 85%, greater than about 80%, greater than about 75%, greater than about 70%, greater than about 65%, greater than about 60%, greater than about 55%, greater than about 50%, greater than about 45%, greater than about 40%, greater than about 35%, greater than about 30%, greater than about 25%, greater than about 20%, greater than about 15%, or greater than about 10% by volume of the decellularized, milled, digested, lyophilized, and/or washed tendon tissue.

In an aspect, the decellularizing step comprises exposing the minced tendon tissue specimen to a solution comprising one or more components selected from the group consisting of a chaotropic salt, a non-ionic detergent, a zwitterionic detergent, a cationic detergent, an anionic detergent, or combinations thereof. In some aspects, the decellularizing step comprises one or more freeze/thaw cycles. In some aspects, the decellularizing step further comprises treatment with DNAase and/or RNAase. In some aspects, the decellularizing step further comprises one or more washes in a balance salt solution, for example, phosphate buffered saline of Hank's balanced salt solution.

In some embodiments, the minced tendon tissue specimen is rinsed in ultrapure water and then decellularized using a solution comprising 1% w/v sodium dodecyl sulfate (SDS) with using moderate stirring. In some embodiments, the moderate stirring is intermittent.

In another aspect, the minced tendon tissue specimen is decellularized using a solution comprising one or more of an ionic detergent, a nonionic detergent, an anionic detergent, or a cationic detergent. In some aspects, the decellularization solution further comprises a chaotropic salt. In some embodiments, the chaotropic salt is urea. In some embodiments, the decellularization solution comprises 0.5 M urea to 8 M urea. In some embodiments, the decellularization solution comprises 2 M to 5 M urea. In some embodiments the decellularization solution comprises about 3 M urea.

In some aspects, the decellularization solution comprises a surfactant, and a chaotropic salt. In some aspects, the decellularization solution further comprises an antifoam agent, for example, Antifoam 204.

In another aspect, the process further comprises a step to precipitate cellular proteins, the process further comprising treating the minced tendon tissue specimen with a concentrated cosmotropic solution. In some embodiments, the concentrated cosmotropic solution is ammonium sulfate. Cosmotropic salting out is accomplished, for example, according to the methods summarized by Wingfield, Curr. Protoc. Protein Sci., APPENDIX 3: Appendix-3F (2001).

Mincing may be accomplished using methods know to the art, for example, first removing sheath, adipose and synovial tissue from the tendon tissue specimen. Then, the tendon tissue specimen is minced into pieces roughly 1 to 4 mm³ in size, then washed with phosphate-buffered saline (PBS).

In an aspect, the stopping and neutralizing step comprises stopping and neutralizing with a solution comprising one or more protease inhibitor selected from the group consisting of TAPI-0, TAPI-1, TAPI-2, marimastat, phosphoramidon, luteolin, PMSF, pepstatin A, leupeptin, E-64, sodium orthovanadate, or combinations thereof.

Decellularization may be monitored by methods known to the art, including, sectioning decellularized specimens and control specimens (i.e. untreated samples of starting donor tendon tissue), then staining with hematoxylin-eosin staining and Masson-Goldner's trichrome stain to detect the cellular components and collagen fibrous structures, respectively. DNA maybe extracted from decellularized samples and untreated, starting samples; decellularized samples should have at least 4-fold less DNA recovered for comparable starting weights. See, e.g., Seif-Naraghi et al., Acta Biomater. 8:3695-3703 (2012).

A decellularized tissue has the extracellular matrix (ECM) component of all or most regions of the tissue, including ECM components of the vascular tree. ECM components can include any one or any combination of the following: fibronectin, fibrillin, laminin, elastin, members of the collagen family (e.g., collagen I, III, and IV), ECM associated growth proteins including growth factors and cytokines, glycosaminoglycans, ground substance, reticular fibers and thrombospondin, which can remain organized as defined structures such as the basal lamina. Successful decellularization can be defined as the absence of detectable myofilaments, endothelial cells, smooth muscle cells, and nuclei in histologic sections using standard histological staining procedures or removal of over 97% of detectable DNA (e.g., as measured by fluorometric assay). Residual cell debris may be removed from the decellularized tissue.

The morphology and the architecture of the ECM can be maintained during and following the process of decellularization. “Morphology” as used herein refers to the overall shape of the of the ECM, while “architecture” as used herein refers to the exterior surface, the interior surface, and the ECM therebetween. The morphology and architecture of the ECM may be examined visually and/or histologically.

One or more compounds can be applied in or on a decellularized tissue to, for example, preserve the decellularized tissue, or to prepare the decellularized tissue for recellularization or integration or implant into a host. Such compounds include, but are not limited to, one or more growth factors (e.g., VEGF, DKK-1, FGF, BMP-1, BMP-4, SDF-1, IGF, and HGF), immune modulating agents (e.g., cytokines, glucocorticoids, IL2R antagonist, leucotriene antagonists), and/or factors that modify the coagulation cascade (e.g., aspirin, heparin-binding proteins, and heparin). In addition, a decellularized tissue may be further treated with, for example, irradiation (e.g., UV, gamma) to reduce or eliminate the presence of any type of microorganism remaining on or in a decellularized tissue.

In some aspects, the disclosure provides a method of making a decellularized tendon matrix (DTM) composition, the composition made using the method further comprises retaining at least 100, at least 99, at least 98, at least 97, at least 96, at least 95, at least 94, at least 93, at least 92, at least 91, at least 90% of the growth factors present in the minced tendon tissue. In some aspects, composition made using method of making a decellularized tendon matrix (DTM) composition, the composition made using the method further comprises retaining at least 85%, at least 80%, at least 75%, at least 70%, at least 65%, at least 60%, at least 55%, at least 50%, at least 45%, or at least 40% of the growth factors present in the minced tendon tissue. In some aspects, the disclosure provides a method of making a decellularized tendon matrix (DTM) composition, the composition made using the method further comprises retaining at least 99%, at least 98%, at least 97%, at least 96%, at least 95%, at least 94%, at least 93%, at least 92%, at least 91%, at least 90%, at least 89%, at least 88%, at least 87%, at least 86%, at least 85%, at least 84%, at least 83%, at least 82%, at least 81%, at least 80%, at least 79%, at least 78%, at least 77%, at least 76%, at least 75%, at least 74%, at least 73%, at least 72%, at least 71%, at least 70%, at least 69%, at least 68%, at least 67%, at least 66%, at least 65%, at least 64%, at least 63%, at least 62%, at least 61%, at least 60%, at least 59%, at least 58%, at least 57%, at least 56% at least 55%, at least 54%, at least 53%, at least 52%, at least 51%, at least 50%, at least 49%, at least 48%, at least 47%, at least 46%, at least 45%, at least 44%, at least 43%, at least 42%, at least 41%, at least 40%, at least 39%, at least 38%, at least 37%, at least 36%, at least 35%, at least 34%, at least 33%, at least 32%, at least 31%, at least 31%, at least 30%, at least 29%, at least 28%, at least 27%, at least 26% at least 25%, at least 24%, at least 23%, at least 22%, at least 21%, at least 20%, at least 19%, at least 18%, at least 17%, at least 16%, at least 15%, at least 14%, at least 13%, at least 12%, at least 11%, or at least 10% of the growth factors present in the minced tendon tissue. In some aspects, the disclosure provides a method of making a decellularized tendon matrix (DTM) composition, wherein the composition made using the method retained between about 70% and about 100% of the growth factors present in the minced tendon tissue before decellularizing. In some aspects, the disclosure provides a method of making a decellularized tendon matrix (DTM) composition, wherein the composition made using the method retained between about 70% and about 75% of the growth factors present in the minced tendon tissue before decellularizing. In some aspects, the disclosure provides a method of making a decellularized tendon matrix (DTM) composition, wherein the composition made using the method retained between about 75% and about 80% of the growth factors present in the minced tendon tissue before decellularizing. In some aspects, the disclosure provides a method of making a decellularized tendon matrix (DTM) composition, wherein the composition made using the method retained between about 80% and about 85% of the growth factors present in the minced tendon tissue before decellularizing. In some aspects, the disclosure provides a method of making a decellularized tendon matrix (DTM) composition, wherein the composition made using the method retained between about 85% and about 90% of the growth factors present in the minced tendon tissue before decellularizing. In some aspects, the disclosure provides a method of making a decellularized tendon matrix (DTM) composition, wherein the composition made using the method retained between about 90% and about 95% of the growth factors present in the minced tendon tissue before decellularizing. In some aspects, the disclosure provides a method of making a decellularized tendon matrix (DTM) composition, wherein the composition made using the method retained between about 95% and about 100% of the growth factors present in the minced tendon tissue before decellularizing. In some aspects, the disclosure provides a method of making a decellularized tendon matrix (DTM) composition, wherein the composition made using the method retained between about 75% and about 95% of the growth factors present in the minced tendon tissue before decellularizing. In some aspects, the disclosure provides a method of making a decellularized tendon matrix (DTM) composition, wherein the composition made using the method retained between about 70% and about 80% of the growth factors present in the minced tendon tissue before decellularizing. In some aspects, the disclosure provides a method of making a decellularized tendon matrix (DTM) composition, wherein the composition made using the method retained between about 80% and about 90% of the growth factors present in the minced tendon tissue before decellularizing. In some embodiments, the growth factors are selected from the group consisting of IGF-1, TGF-β, PDGF, VEGF, bFGF, GDF-5, GDF-6, GDF-7, HGF, and combinations thereof. In some embodiments, the growth factors include at least TGF-β.

In some aspects, the disclosure provides a method of making a decellularized tendon matrix (DTM) composition, the method further comprises retaining at least 90% of the cytokines present in the minced tendon tissue, wherein the growth factors are selected from the group consisting of IGF-1, TGF-β, PDGF, VEGF, bFGF, GDF-5, GDF-6, GDF-7, HGF, and combinations thereof. In some aspects, method of making a decellularized tendon matrix (DTM) composition, the method further comprises retaining at least 99%, at least 98%, at least 97%, at least 96%, at least 95%, at least 94%, at least 93%, at least 92%, at least 91%, at least 90%, at least 89%, at least 88%, at least 87%, at least 86%, at least 85%, at least 84%, at least 83%, at least 82%, at least 81%, at least 80%, at least 79%, at least 78%, at least 77%, at least 76%, at least 75%, at least 74%, at least 73%, at least 72%, at least 71%, at least 70%, at least 69%, at least 68%, at least 67%, at least 66%, at least 65%, at least 64%, at least 63%, at least 62%, at least 61%, at least 60%, at least 59%, at least 58%, at least 57%, at least 56% at least 55%, at least 54%, at least 53%, at least 52%, at least 51%, at least 50%, at least 49%, at least 48%, at least 47%, at least 46%, at least 45%, at least 44%, at least 43%, at least 42%, at least 41%, at least 40%, at least 39%, at least 38%, at least 37%, at least 36%, at least 35%, at least 34%, at least 33%, at least 32%, at least 31%, at least 31%, at least 30%, at least 29%, at least 28%, at least 27%, at least 26% at least 25%, at least 24%, at least 23%, at least 22%, at least 21%, at least 20%, at least 19%, at least 18%, at least 17%, at least 16%, at least 15%, at least 14%, at least 13%, at least 12%, at least 11%, or at least 10% of the growth factors present in the minced tendon tissue, wherein the growth factors are selected from the group consisting of IGF-1, TGF-(3, PDGF, VEGF, bFGF, GDF-5, GDF-6, GDF-7, HGF, and combinations thereof.

In some aspects, the disclosure provides a method of making a decellularized tendon matrix (DTM) composition, the method further comprises retaining at least 90% of TGF-present in the minced tendon tissue. In some aspects, the disclosure provides a method of making a decellularized tendon matrix (DTM) composition, the method further comprises retaining at least 95% of TGF-β present in the minced tendon tissue. In some aspects, the disclosure provides a method of making a decellularized tendon matrix (DTM) composition, the method further comprises retaining at least 99% of TGF-β present in the minced tendon tissue. In some aspects, method of making a decellularized tendon matrix (DTM) composition, the method further comprises retaining at least 99%, at least 98%, at least 97%, at least 96%, at least 95%, at least 94%, at least 93%, at least 92%, at least 91%, at least 90%, at least 89%, at least 88%, at least 87%, at least 86%, at least 85%, at least 84%, at least 83%, at least 82%, at least 81%, at least 80%, at least 79%, at least 78%, at least 77%, at least 76%, at least 75%, at least 74%, at least 73%, at least 72%, at least 71%, at least 70%, at least 69%, at least 68%, at least 67%, at least 66%, at least 65%, at least 64%, at least 63%, at least 62%, at least 61%, at least 60%, at least 59%, at least 58%, at least 57%, at least 56% at least 55%, at least 54%, at least 53%, at least 52%, at least 51%, at least 50%, at least 49%, at least 48%, at least 47%, at least 46%, at least 45%, at least 44%, at least 43%, at least 42%, at least 41%, at least 40%, at least 39%, at least 38%, at least 37%, at least 36%, at least 35%, at least 34%, at least 33%, at least 32%, at least 31%, at least 31%, at least 30%, at least 29%, at least 28%, at least 27%, at least 26% at least 25%, at least 24%, at least 23%, at least 22%, at least 21%, at least 20%, at least 19%, at least 18%, at least 17%, at least 16%, at least 15%, at least 14%, at least 13%, at least 12%, at least 11%, or at least 10% by weight of TGF-β in the native tendon.

In some aspects, method of making a decellularized tendon matrix (DTM) composition, the method further comprises retaining at least 85%, at least 80%, at least 75%, at least 70%, at least 65%, at least 60%, at least 55%, at least 50%, at least 45%, or at least 40% of the growth factors present in the minced tendon tissue, wherein the growth factors are selected from the group consisting of IGF-1, TGF-β, PDGF, VEGF, bFGF, GDF-5, GDF-6, GDF-7, HGF, and combinations thereof.

In some aspects, method of making a decellularized tendon matrix (DTM) composition, the method further comprises increasing the concentration of growth factors present in the decellularized tissue or DTM by at least 500%, at least 250%, at least 200%, at least 150%, at least 100%, at least 95%, at least 90%, at least 85%, at least 80%, at least 75%, at least 70%, at least 65%, at least 60%, at least 55%, at least 50%, at least 45%, at least 40%, at least 35%, at least 30%, at least 25%, at least 20%, at least 15%, at least 10%, at least 5%, wherein the growth factors are selected from the group consisting of IGF-1, TGF-β, PDGF, VEGF, bFGF, GDF-5, GDF-6, GDF-7, HGF, and combinations thereof.

In an aspect, the composition retains 2 or more of the above growth factors, 3 or more of the above growth factors, 4 or more of the above growth factors, 5 or more of the above growth factors, 6 or more of the above growth factors, 7 or more of the above growth factors. In an aspect, the composition retains HGF and one or more growth factors selected from the group consisting of IGF-1, TGF-β, PDGF, VEGF, bFGF, GDF-5, GDF-6, and GDF-7. In an aspect the composition retains IGF-1 and HGF.

In an aspect, the DTM composition further comprises retaining at least at least 85%, at least 84%, at least 83%, at least 82%, at least 81%, at least 80%, at least 79%, at least 78%, at least 77%, at least 76%, at least 75%, at least 74%, at least 73%, at least 72%, at least 71%, at least 70%, at least 69%, at least 68%, at least 67%, at least 66%, at least 65%, at least 64%, at least 63%, at least 62%, at least 61%, at least 60%, at least 59%, at least 58%, at least 57%, at least 56% at least 55%, at least 54%, at least 53%, at least 52%, at least 51%, at least 50%, at least 49%, at least 48%, at least 47%, at least 46%, at least 45%, at least 44%, at least 43%, at least 42%, at least 41%, at least 40%, at least 39%, at least 38%, at least 37%, at least 36%, at least 35%, at least 34%, at least 33%, at least 32%, at least 31%, at least 31%, at least 30%, at least 29%, at least 28%, at least 27%, at least 26% at least 25%, at least 24%, at least 23%, at least 22%, at least 21%, at least 20%, at least 19%, at least 18%, at least 17%, at least 16%, at least 15%, at least 14%, at least 13%, at least 12%, at least 11%, or at least 10% of the IGF-1 and HGF present in the minced tendon tissue.

In some aspects, the disclosure provides a method of making a decellularized tendon matrix (DTM) composition, the method further comprises removing at least 90% of cellular material present in the minced tendon tissue. In some aspects, the disclosure provides a method of making a decellularized tendon matrix (DTM) composition, the method further comprises removing at least 95% of cellular material present in the minced tendon tissue. In some aspects, the disclosure provides a method of making a decellularized tendon matrix (DTM) composition, the method further comprises removing at least 99% of cellular material present in the minced tendon tissue. In some aspects, method of making a decellularized tendon matrix (DTM) composition, the method further comprises removing at least 99%, at least 98%, at least 97%, at least 96%, at least 95%, at least 94%, at least 93%, at least 92%, at least 91%, at least 90%, at least 89%, at least 88%, at least 87%, at least 86%, at least 85%, at least 84%, at least 83%, at least 82%, at least 81%, at least 80%, at least 79%, at least 78%, at least 77%, at least 76%, at least 75%, at least 74%, at least 73%, at least 72%, at least 71%, at least 70%, at least 69%, at least 68%, at least 67%, at least 66%, at least 65%, at least 64%, at least 63%, at least 62%, at least 61%, at least 60%, at least 59%, at least 58%, at least 57%, at least 56% at least 55%, at least 54%, at least 53%, at least 52%, at least 51%, at least 50%, at least 49%, at least 48%, at least 47%, at least 46%, at least 45%, at least 44%, at least 43%, at least 42%, at least 41%, at least 40%, at least 39%, at least 38%, at least 37%, at least 36%, at least 35%, at least 34%, at least 33%, at least 32%, at least 31%, at least 31%, at least 30%, at least 29%, at least 28%, at least 27%, at least 26% at least 25%, at least 24%, at least 23%, at least 22%, at least 21%, at least 20%, at least 19%, at least 18%, at least 17%, at least 16%, at least 15%, at least 14%, at least 13%, at least 12%, at least 11%, or at least 10% by weight of cellular material in the native tendon. In certain embodiments, the disclosure provides a method of making a decellularized tendon matrix (DTM) composition, and the DTM is substantially free of cellular material. In certain embodiments, the disclosure provides a method of making a decellularized tendon matrix (DTM) composition, and the DTM is substantially free of TGF-β producing cells.

In some aspects, the disclosure provides a method of making a decellularized tendon matrix (DTM) composition, the method further comprises removing at least 90% of nucleic acids (e.g., DNA or RNA) present in the minced tendon tissue. In some aspects, the disclosure provides a method of making a decellularized tendon matrix (DTM) composition, the method further comprises removing at least 95% of nucleic acids (e.g., DNA or RNA) present in the minced tendon tissue. In some aspects, the disclosure provides a method of making a decellularized tendon matrix (DTM) composition, the method further comprises removing at least 99% of nucleic acids (e.g., DNA or RNA) present in the minced tendon tissue. In some aspects, method of making a decellularized tendon matrix (DTM) composition, the method further comprises removing at least 99%, at least 98%, at least 97%, at least 96%, at least 95%, at least 94%, at least 93%, at least 92%, at least 91%, at least 90%, at least 89%, at least 88%, at least 87%, at least 86%, at least 85%, at least 84%, at least 83%, at least 82%, at least 81%, at least 80%, at least 79%, at least 78%, at least 77%, at least 76%, at least 75%, at least 74%, at least 73%, at least 72%, at least 71%, at least 70%, at least 69%, at least 68%, at least 67%, at least 66%, at least 65%, at least 64%, at least 63%, at least 62%, at least 61%, at least 60%, at least 59%, at least 58%, at least 57%, at least 56% at least 55%, at least 54%, at least 53%, at least 52%, at least 51%, at least 50%, at least 49%, at least 48%, at least 47%, at least 46%, at least 45%, at least 44%, at least 43%, at least 42%, at least 41%, at least 40%, at least 39%, at least 38%, at least 37%, at least 36%, at least 35%, at least 34%, at least 33%, at least 32%, at least 31%, at least 31%, at least 30%, at least 29%, at least 28%, at least 27%, at least 26% at least 25%, at least 24%, at least 23%, at least 22%, at least 21%, at least 20%, at least 19%, at least 18%, at least 17%, at least 16%, at least 15%, at least 14%, at least 13%, at least 12%, at least 11%, or at least 10% by weight of nucleic acids (e.g., DNA or RNA) in the native tendon. In certain embodiments, the disclosure provides a method of making a decellularized tendon matrix (DTM) composition, and the DTM is substantially free of nucleic acids (e.g., DNA or RNA).

A variety of methods are known to the art, for example, those summarized by Gilpin and Yang, Biomed. Res. Int. 2017: 9831534 (2017). Many methods comprise aggressive detergent extractions and prolonged treatment with promiscuous proteases, for example, pepsin, at extreme, non-physiological pHs. The methods and processes of the present disclosure differ from those known to the art, by employing less promiscuous proteases that are active at physiological pHs. Without being bound by theory, the methods and processes of the present disclosure are less protein denaturing and preserve more functional growth factors in the decellularized tendon matrix. In some aspects, MMP2, MMP9, MMP14, or combinations thereof, are used to prepare decellularized tendon matrix compositions of the disclosure. The target cleavage sites for the MMP family, including MMP2, MMP9, and MMP14, have been mapped using a whole proteome approach by Eckhard et al., Data Brief 7: 299-310 (2017).

### DTM Hydrogels

In another aspect, the present disclosure provides for decellularized tendon matrix hydrogels. Hydrogels may be produced using the intrinsic polymerization capability of pepsin-processed monomeric collagen by manipulating the temperature or pH. These approaches are well known, yet somewhat unpredictable, for example, Drake et al., Biochemistry 5:301-312 (1966) details the production of polymerizable proteolytic fragments of collagen. Other methods, such as those taught by Bahney et al., FASEB J, 25:1486-1496 (2011) and Ungerleider et al., Methods, 84:53-59 (2015), also well known. These well-known methods are particularly unpredictable when applied to protein-rich extracellular matrix tissues.

More reliable and better controlled crosslinking may be effected by using carbodiimide cross linker chemistry. In some embodiments, hydrogels are produced by mixing DTM compositions and reacting with a carboxyl-reactive cross linker, for example, 1-ethyl-3-(3-dimethylaminopropyl)carbodiimide, “EDC.” EDC crosslinking is most efficient in acidic (e.g. about pH 4.5) conditions and best performed in buffers without extraneous carboxyls and amines. IVIES buffer (4-morpholinoethanesulfonic acid) is a suitable carbodiimide reaction buffer. Phosphate buffers and neutral pH (up to 7.2) conditions are compatible with the reaction chemistry, but with lower efficiency; increasing the amount of EDC in a reaction solution can readily compensate for any reduced efficiency. EDC is mixed 1:1 with N-hydroxysuccinimide (NHS) or its water-soluble analog (Sulfo-NHS) is to further improve crosslinking. EDC couples NHS to carboxyls, forming an NHS ester that is considerably more stable than the O-acylisourea intermediate while allowing for efficient conjugation to primary amines at physiologic pH.

In another aspect, a DTM hydrogel is formed by reconstituting DTM in a sterile pharmaceutically acceptable solution for injection.

### Pharmaceutical Compositions for Injection

In one aspect, the disclosure provides a pharmaceutical composition for use in the repair or treatment of tendon tears. In a preferred embodiment, the disclosure provides pharmaceutical composition comprising a DTM hydrogel, that is applied directly to the location of tendon damage. In an aspect, the location of tendon damage is a first degree tear. In another aspect, the location of tendon damage is a second degree tear. In an aspect, the location of tendon damage is a third degree tear.

The pharmaceutical compositions are typically formulated to provide a therapeutically effective amount of a DTM hydrogel, the pharmaceutical composition further comprising one or more pharmaceutically acceptable excipients, carriers, including inert solid diluents and fillers, diluents, including sterile aqueous solution and various organic solvents, permeation enhancers, solubilizers and adjuvants.

In making the compositions of this disclosure, compositions comprising decellularized tendon matrix can also comprise an excipient. Some examples of suitable excipients include lactose, dextrose, sucrose, sorbitol, mannitol, starches, gum acacia, calcium phosphate, alginates, tragacanth, gelatin, calcium silicate, microcrystalline cellulose, PEG, polyvinylpyrrolidone, cellulose, water, sterile saline, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of an active ingredient (e.g., a growth factor) after implant into the patient by employing procedures known in the art.

In some cases, the pharmaceutical compositions described herein may comprise an excipient that can provide long term preservation, bulk up a formulation that contains potent active ingredients, facilitate drug absorption, reduce viscosity, add flavoring, or enhance the solubility of the pharmaceutical composition. Non-limiting examples of excipients can include anti-adherents, binders (e.g., sucrose, lactose, starches, cellulose, gelatin, or polyethylene glycol), coatings (e.g., hydroxypropyl methylcellulose or gelatin), disintegrants, glidants, lubricants, or preservatives (e.g., acids, esters, phenols, mercurial compounds, or ammonium compounds). A pharmaceutical composition of the present disclosure can comprise about 1%, 2%, 3%, 4%, 5%, 6%, 7%, 8%, 9%, 10%, 15%, 20%, 25%, 30%, 35%, 40%, 45%, 50%, or greater than about 50% of the excipient by weight or by volume. For example, a pharmaceutical composition can comprise 5% of an excipient by volume. In another example, a pharmaceutical composition can comprise 8% of an excipient by weight. It is contemplated that one or more vehicles may be chosen based on the active ingredient in the pharmaceutical composition.

In certain embodiments, a pharmaceutical composition of the present disclosure can comprise one or more solubilizers. As used herein, “solubilizers” include compounds such as triacetin, triethylcitrate, ethyl oleate, ethyl caprylate, sodium lauryl sulfate, sodium docusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrrns, ethanol, n-butanoL isopropyl alcohol, cholesterol, bile salts, polyethylene glycol 200-600, glycofurol, transcutol, propylene glycol, and dimethyl isosorbide and the like. A pharmaceutical composition of the present disclosure can comprise about 1%, 2%, 3%, 4%, 5%, 6%, 7%, 8%, 9%, 10%, 15%, 20%, 25%, 30%, 35%, 40%, 45%, 50%, or greater than about 50% of the solubilizer by weight or by volume. For example, a pharmaceutical composition can comprise 10% of a solubilizer by volume. In another example, a pharmaceutical composition can comprise 5% of a solubilizer by weight.

In some embodiments, the compositions comprise a stabilizing agent. In some embodiments, stabilizing agent is selected from, for example, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, and combinations thereof. In some embodiments, amide analogues of stabilizers are also used. Other useful compositions include one or more antioxidants to enhance chemical stability where required. Suitable antioxidants include, by way of example only, ascorbic acid and sodium metabisulfite. In one embodiment, antioxidants are selected from metal chelating agents, thiol containing compounds and other general stabilizing agents.

Still other useful compositions include one or more surfactants to enhance physical stability or for other purposes. Suitable nonionic surfactants include polyoxyethylene fatty acid glycerides and vegetable oils, polyoxyethylene, hydrogenated castor oil, polyoxyethylene alkylethers, alkylphenyl ethers, octoxynol 10, and octoxynol 40.

In some embodiments, the compositions disclosed herein comprise preservatives. Suitable preservatives for use in the compositions described herein include, but are not limited to benzoic acid, boric acid, p-hydroxybenzoates, phenols, chlorinated phenolic compounds, alcohols, quarternary compounds, quaternary ammonium compounds (e.g. benzalkonium chloride, cetyltrimethylammonium bromide or cetylpyridinium chloride), stabilized chlorine dioxide, mercurials (e.g. merfen or thiomersal), or mixtures thereof. In some embodiments, the preservative is methyl paraben. In some embodiments, the methyl paraben is at a concentration of about 0.05% to about 1.0%, about 0.1% to about 0.2% by weight or by volume.

In some embodiments, a composition of the present disclosure can comprise a base, and the base can include sodium stearyl fumarate, diethanolamine cetyl sulfate, isostearate, polyethoxylated castor oil, benzalkonium chloride, nonoxyl 10, octoxynol 9, sodium lauryl sulfate, sorbitan esters (sorbitan monolaurate, sorbitan monooleate, sorbitan monopalmitate, sorbitan monostearate, sorbitan sesquioleate, sorbitan trioleate, sorbitan tristearate, sorbitan laurate, sorbitan oleate, sorbitan palmitate, sorbitan stearate, sorbitan dioleate, sorbitan sesqui-isostearate, sorbitan sesquistearate, sorbitan tri-isostearate), lecithin, pharmaceutical acceptable salts thereof, combinations thereof, or derivatives thereof.

In an embodiment, the concentration of decellularized tendon matrix (DTM) in the DTM hydrogel pharmaceutical compositions is selected from the group consisting of about 0.2 mg/mL to 20 mg/mL; 0.2 mg/mL to 19 mg/mL; 0.2 mg/mL to 18 mg/mL; 0.2 mg/mL to 17 mg/mL; 0.2 mg/mL to 16 mg/mL; 0.2 mg/mL to 15 mg/mL; 0.2 mg/mL to 14 mg/mL; 0.2 mg/mL to 13 mg/mL; 0.2 mg/mL to 12 mg/mL; 0.2 mg/mL to 11 mg/mL; 0.2 mg/mL to 10 mg/mL; 0.2 mg/mL to 9 mg/mL; 0.2 mg/mL to 8 mg/mL; 0.2 mg/mL to 7 mg/mL; 0.2 mg/mL to 6 mg/mL; 0.3 mg/mL to 6 mg/mL; 0.4 mg/mL to 6 mg/mL; 0.5 mg/mL to 6 mg/mL; 0.6 mg/mL to 6 mg/mL; 0.7 mg/mL to 6 mg/mL; 0.8 mg/mL to 6 mg/mL; 0.9 mg/mL to 6 mg/mL; 1 mg/mL to 6 mg/mL; 2 mg/mL to 6 mg/mL; 3 mg/mL to 6 mg/mL; about 3 mg/mL; about 4 mg/mL; about 5 mg/mL; and about 6 mg/mL.

In an embodiment, the concentration of decellularized tendon matrix (DTM) in the DTM hydrogel pharmaceutical compositions is selected from the group consisting of about 1.0 mg/mL to 6 mg/mL; 1.1 mg/mL to 6 mg/mL; 1.2 mg/mL to 6 mg/mL; 1.3 mg/mL to 6 mg/mL; 1.4 mg/mL to 6 mg/mL; 1.5 mg/mL to 6 mg/mL; 1.6 mg/mL to 6 mg/mL; 1.7 mg/mL to 6 mg/mL; 1.8 mg/mL to 6 mg/mL; 1.9 mg/mL to 6 mg/mL; 2.0 mg/mL to 6 mg/mL; 2.1 mg/mL to 6 mg/mL; 2.2 mg/mL to 6 mg/mL; 2.3 mg/mL to 6 mg/mL; 2.4 mg/mL to 6 mg/mL; 2.5 mg/mL to 6 mg/mL; 2.6 mg/mL to 6 mg/mL; 2.7 mg/mL to 6 mg/mL; 2.8 mg/mL to 6 mg/mL; 2.9 mg/mL to 6 mg/mL; 3.0 mg/mL to 6 mg/mL; 3.1 mg/mL to 6 mg/mL; 3.2 mg/mL to 6 mg/mL; 3.3 mg/mL to 6 mg/mL; 3.4 mg/mL to 6 mg/mL; 3.5 mg/mL to 6 mg/mL; 3.6 mg/mL to 6 mg/mL; 3.7 mg/mL to 6 mg/mL; 3.8 mg/mL to 6 mg/mL; 3.9 mg/mL to 6 mg/mL; 4.0 mg/mL to 6 mg/mL; 4.1 mg/mL to 6 mg/mL; 4.2 mg/mL to 6 mg/mL; 4.3 mg/mL to 6 mg/mL; 4.4 mg/mL to 6 mg/mL; 4.5 mg/mL to 6 mg/mL; 4.6 mg/mL to 6 mg/mL; 4.7 mg/mL to 6 mg/mL; 4.8 mg/mL to 6 mg/mL; 4.9 mg/mL to 6 mg/mL; 5.0 mg/mL to 6 mg/mL; 5.1 mg/mL to 6 mg/mL; 5.2 mg/mL to 6 mg/mL; 5.3 mg/mL to 6 mg/mL; 5.4 mg/mL to 6 mg/mL; 5.5 mg/mL to 6 mg/mL; 5.6 mg/mL to 6 mg/mL; 5.7 mg/mL to 6 mg/mL; 5.8 mg/mL to 6 mg/mL; and 6 mg/mL.

In an embodiment, the DTM hydrogel percentage (%) in the pharmaceutical composition is selected from the group consisting of about provided in the pharmaceutical compositions of the disclosure is independently less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, suspending agents, binders, fillers, plasticizers, lubricants, and mixtures thereof.

Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

The composition can further comprise a peptide. The composition can further comprise a protein. The composition can further comprise an amino acid. The composition can further comprise water.

The composition can further comprise at least one growth factor. In some cases, the at least one growth factor can comprise insulin-like growth factor-1, insulin-like growth factor binding protein-3, vascular endothelial growth factor (VEGF), hepatocyte growth factor (HGF), placenta growth factor (PLGF), or any combination thereof. The at least one growth factors can enhance viability, enhance stability of product, differentiation of cells, preservation of sternness, reduce anti-inflammatory, or any combinations thereof. The at least one growth factor can be added to the composition. The at least one growth factor can be added to a subcomponent of the composition. The at least one growth factor can be added to a viscosity modifying component, a plurality of isolated stem cells, an isolated inductive component, an isolated scaffolding component, or any combinations thereof. For example, the at least one growth factor can be added to a composition of the present disclosure comprising a decellularized tendon matrix to enhance host tissue integration with the composition upon transplant into a host. The at least one growth factor can be added prior to forming the composition. The at least one growth factor can be added after forming the composition.

The composition can further comprise at least one of: chemokine ligand 2, macrophage inflammatory protein-1 (MIP-1) alpha, MIP-1 beta, MIP-2, beta-chemokine ligand-5, beta-chemokine ligand-20, alpha-chemokine ligand-14, lipopolysaccharide-induced alpha-chemokine, Granulocyte-macrophage colony-stimulating factor, interleukin IL-1 beta, phorbol myristate acetate, epidermal growth factor, fibroblast growth factor, vascular endothelial growth factor, connective tissue growth factor, platelet-derived growth factor, insulin-like growth factor, nerve growth factor, hepatocyte growth factor, colony-stimulating factor, stem cell factor, keratinocyte growth factor, granulocyte colony-stimulating factor, granulocyte macrophage colony-stimulating factor, glial derived neurotrophic factor, ciliary neurotrophic factor, endothelial-monocyte activating polypeptide, epithelial neutrophil activating peptide, erythropoietin, bone morphogenetic protein, brain-derived neurotrophic factor, transforming growth factor beta, tumor necrosis factor, or any combination thereof. The composition can further comprise at least one hormone. In some cases, the at least one hormone can be prolactin or leptin.

In some cases, there can be six major growth factor families (EGF, FGF, IGF, PDGF, TGF, and VEGF) associated with healing. Examples of such growth factors can include, but are not limited to, platelet derived growth factor (PDGF-A, PDGF-B, PDGF-C, and PDGF-D), insulin-like growth factor I and II (IGF-I and IGF-II), acidic and basic fibroblast growth factor (aFGF and bFGF), alpha and beta transforming growth factor (TGF-a and TGF-β (for example, TGF-beta 1, TGF beta 2, TGF beta 3)), epidermal growth factor (EGF), and others. These growth factors can stimulate mitosis of one or more of the cells involved in healing and can be combined.

Other positive angiogenesis agents co-administered with the compositions disclosed herein can include, but are not limited to, e.g., HGF, TNF-α, angiogenin, IL-8, etc. Still further examples of additional agents can include Platelet-derived growth factor (PDGF) (e.g., Becaplermin (rhPDGF-BB) such as REGRANEX®, adenosine-A2A receptor agonists; keratinocyte growth factor (KGF-2, repifermin; lactoferrin (LF); thymosine beta-4 (T134); thrombin-derived activating receptor peptide (TP508; CHRYSALIN®; adenoviral vector encoding platelet-derived growth factor (PDGF-B); autologous bone marrow stem cells (BMSC); and, engineered living tissue grafts (e.g., Apligraf, etc.). Antibiotic and antiseptic ulcer agents can also be combined. Immunosuppressive treatment (e.g., corticosteroids, radiation therapy, chemotherapy) can be combined with the compositions disclosed herein.

A person having skill in the art will appreciate that additional agents can be co-administered with the composition disclosed herein or administered separately.

Compositions of the disclosure can comprise, in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, et al., eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

The compositions of the disclosure may also be delivered via an impregnated or coated device such as a suture, for example, suture anchor. Such a method of administration may, for example, aid in the prevention or amelioration of tendon damage or injury. A composition of the disclosure may be administered, for example, by local delivery from the suture or suture anchor. In some embodiments, a compound of the disclosure is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters; and Polyether ether ketone (PEEK). Metals or biocomposite materials, for example poly(lactic acid) (PLA) and beta-tricalcium phosphate (β-TCP) are also suitable. PLA/hydroxyapatite may also be used, see, e.g. Dorozhkin, Biomatter, 1:3-56 (2011). Suitable matrices may be nondegrading or may degrade with time, releasing the compound or compounds. Compositions of the disclosure may be applied directly to the sites of tendon injury and/or directly to sites of tendon damage. In some aspects, compositions of the disclosure are applied adjacent to sites of tendon injury and/or adjacent to sites of tendon damage. In another aspect, compositions of the disclosures are applied to tendons in need of regeneration.

DTM hydrogels may be applied to the surface of the suture, suture anchor, or medical device by various methods such as dip/spin coating, spray coating, dip-coating, and/or brush-coating. The compounds may be applied in a solvent and the solvent may be allowed to evaporate, thus forming a layer of hydrogel onto the suture, suture anchor, or medical device. Alternatively, the compound may be located in the body of the suture, suture anchor, or medical device, for example in microchannels or micropores. When implanted, the compound diffuses out of the body of the suture, suture anchor, or medical device to contact the tendon. Such suture, suture anchor, or medical devices may be prepared by dipping a suture, suture anchor, or medical device manufactured to contain such micropores or microchannels into a solution of the compositions of the disclosure in a suitable solvent, followed by evaporation of the solvent. Excess hydrogel on the surface of the suture, suture anchor, or medical device may be removed via an additional brief solvent wash. In yet other embodiments, compounds of the disclosure may be covalently linked to a suture, suture anchor, or medical device. A covalent linker may be used which degrades in vivo, leading to the release of the compound of the disclosure. Any bio-labile linkage may be used for such a purpose, such as ester, amide or anhydride linkages.

In some aspects, DTM hydrogels of the disclosure are directly applied to a tendon. In some aspects, DTM hydrogels of the disclosure are directly applied to a tendon using a surgical or medical needle ranging from a 10-gauge needle to a 25-gauge needle. The needle can be 10-gauge, 11-gauge, 12-gauge, 13-gauge, 14-gauge, 15-gauge, 16-gauge, 18-gauge, 20-gauge, 22-gauge, 23-gauge, 24-gauge, or 25-gauge. In some aspects, the needle is 16-gauge to 20-gauge. The viscosity of the DTM hydrogel may be modulated to optimize the composition for delivery through a particular gauge needle; for example, 16-gauge or 20-gauge.

The rheological properties of the DTM hydrogels of the disclosure may be matched to a particular medical or surgical needle gauge for optimal injection. For example, the dynamic viscosity of the DTM hydrogels of the disclosure are between about 0.05 Pa*s to about 1.0 Pa*s.

The disclosure also provides kits. The kits comprise lyophilized DTM composition, and carbodiimide crosslinking reagents, either alone or in combination in suitable packaging, and written material that can include instructions for use, discussion of clinical studies and listing of side effects. In some embodiments, the kit further comprises an applicator for applying the composition to a tendon in need thereof. In some embodiments, the kit further comprises a removable attachment enabling mixing. In an aspect the kit comprises a syringe with lyophilized DTM, a second syringe with an aqueous resuspension buffer, and a mixing connector, that connects the syringes allowing mixing between the two syringes. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials.

### Methods of Treating Tendons

In an aspect, compositions of the disclosure are used to stimulate tendon regeneration, the method comprising: (i) resuspending a DTM composition according to the present disclosure in a pharmaceutically acceptable carrier; and (ii) applying the resuspended DTM composition to a tendon site in need of stimulating tendon regeneration.

In another aspect, a DTM hydrogel is prepared immediately before treating a subject in need thereof, the method comprising: (i) resuspending a DTM composition according to the present disclosure in a pharmaceutically acceptable carrier; (ii) preparing a DTM hydrogel; and (iii) applying the DTM hydrogel to a tendon site in need of stimulating tendon regeneration. In some aspects, the tendon site in need of stimulating tendon regeneration is a first degree tear. In some aspects, the tendon site in need of stimulating tendon regeneration is a second degree tear; in another aspect, the tendon site in need of stimulating tendon regeneration is a third degree tear. In some aspect, the site is a complete tear.

In some aspects, the tendon site in need of stimulating tendon regeneration is a site with an acute injury. In some aspects, the tendon site in need of stimulating tendon regeneration is selected from the group consisting of lateral epicondylitis, Achilles tendonitis, peroneal tendonitis, patellar, quadriceps tendonitis and combinations thereof.

In some aspects, the DTM hydrogel is prepared using carbodiimide chemistry. In some aspects, the DTM hydrogel is prepared by reconstituting the DTM in a pharmaceutically acceptable sterile solution for injection.

In an aspect, DTM compositions of the disclosure are applied to a tendon site in need of repair by single needle injection. In an aspect, application of DTM compositions of the disclosure is image guided. In some aspects, DTM compositions of the disclosure are applied to a tendon site in need of repair using arthroscopy. In another aspect, DTM compositions of the disclosure are applied to a tendon site in need of repair directly, in the course of an open surgical procedure.

In some aspects, compositions of the disclosure are administered to one or more joints via image guided injection. X-ray, computed tomography (CT), or ultrasound are useful imaging methods for guiding joint injections.

The following clauses describe certain embodiments.

Clause 1101. A decellularized tendon matrix (DTM) composition comprising matrix metalloproteinase (MMP) digested tendon tissue, wherein when formulated in a formulation comprising an amount of fluid and about 1 g of composition, the formulation has a complex modulus plateau between about 10³ Pa and about 10^{6.5} Pa. Clause 1102. A decellularized tendon matrix (DTM) composition comprising matrix metalloproteinase (MMP) digested tendon tissue, wherein when formulated in a formulation comprising an amount of fluid and about 1 g of composition, the formulation has a complex modulus plateau between about 10³ Pa and about 10^{3.5} Pa. Clause 1103. A decellularized tendon matrix (DTM) composition comprising matrix metalloproteinase (MMP) digested tendon tissue, wherein when formulated in a formulation comprising an amount of fluid and about 1 g of composition, the formulation has a complex modulus plateau between about 10^{3.5} Pa and about 10⁴ Pa. Clause 1104. A decellularized tendon matrix (DTM) composition comprising matrix metalloproteinase (MMP) digested tendon tissue, wherein when formulated in a formulation comprising an amount of fluid and about 1 g of composition, the formulation has a complex modulus plateau between about 10⁴ Pa and about 10^{4.5} Pa. Clause 1105. A decellularized tendon matrix (DTM) composition comprising matrix metalloproteinase (MMP) digested tendon tissue, wherein when formulated in a formulation comprising an amount of fluid and about 1 g of composition, the formulation has a complex modulus plateau between about 10^{4.5} Pa and about 10⁵ Pa. Clause 1106. A decellularized tendon matrix (DTM) composition comprising matrix metalloproteinase (MMP) digested tendon tissue, wherein when formulated in a formulation comprising an amount of fluid and about 1 g of composition, the formulation has a complex modulus plateau between about 10⁵ Pa and about 10^{5.5} Pa. Clause 1107. A decellularized tendon matrix (DTM) composition comprising matrix metalloproteinase (MMP) digested tendon tissue, wherein when formulated in a formulation comprising an amount of fluid and about 1 g of composition, the formulation has a complex modulus plateau between about 10^{5.5} Pa and about 10⁶ Pa. Clause 1108. A decellularized tendon matrix (DTM) composition comprising matrix metalloproteinase (MMP) digested tendon tissue, wherein when formulated in a formulation comprising an amount of fluid and about 1 g of composition, the formulation has a complex modulus plateau between about 10⁶ Pa and about 10^{6.5} Pa.

Clause 1201. The composition of any one of clauses 1101 to 1108, wherein the amount of fluid is between about 1 ml and about 7 ml. Clause 1202. The composition of any one of clauses 1101 to 1108, wherein the amount of fluid is from more than 0 ml to about 1 ml. Clause 1203. The composition of any one of clauses 1101 to 1108, wherein the amount of fluid is between about 1 ml and about 2 ml. Clause 1204. The composition of any one of clauses 1101 to 1108, wherein the amount of fluid is between about 2 ml and about 3 ml. Clause 1205. The composition of any one of clauses 1101 to 1108, wherein the amount of fluid is between about 3 ml and about 4 ml. Clause 1206. The composition of any one of clauses 1101 to 1108, wherein the amount of fluid is between about 4 ml and about 5 ml. Clause 1207. The composition of any one of clauses 1101 to 1108, wherein the amount of fluid is between about 5 ml and about 6 ml. Clause 1208. The composition of any one of clauses 1101 to 1108, wherein the amount of fluid is between about 6 ml and about 7 ml. Clause 1209. The composition of any one of clauses 1101 to 1108, wherein the amount of fluid is between about 1 ml and about 10 ml. Clause 1210. The composition of any one of clauses 1101 to 1108, wherein the amount of fluid is about 1 ml. Clause 1211. The composition of any one of clauses 1101 to 1108, wherein the amount of fluid is about 2 ml. Clause 1212. The composition of any one of clauses 1101 to 1108, wherein the amount of fluid is about 3 ml. Clause 1213. The composition of any one of clauses 1101 to 1108, wherein the amount of fluid is about 4 ml. Clause 1214. The composition of any one of clauses 1101 to 1108, wherein the amount of fluid is about 5 ml. Clause 1215. The composition of any one of clauses 1101 to 1108, wherein the amount of fluid is about 6 ml. Clause 1216. The composition of any one of clauses 1101 to 1108, wherein the amount of fluid is about 7 ml. Clause 1217. The composition of any one of clauses 1101 to 1108, wherein the amount of fluid is about 8 ml. Clause 1218. The composition of any one of clauses 1101 to 1108, wherein the amount of fluid is about 9 ml. Clause 1219. The composition of any one of clauses 1101 to 1108, wherein the amount of fluid is about 10 ml.

Clause 1301. The composition of any one of clauses 1101 to 1219, wherein when formulated in a formulation comprising about 1 ml of fluid and about 1 g of composition, the formulation has a complex modulus plateau between about 10⁵ Pa and about 10^{6.5} Pa. Clause 1302. The composition of any one of clauses 1101 to 1219, wherein when formulated in a formulation comprising about 1 ml of fluid and about 1 g of composition, the formulation has a complex modulus plateau between about 10⁵ Pa and about 10^{5.1} Pa. Clause 1303. The composition of any one of clauses 1101 to 1219, wherein when formulated in a formulation comprising about 1 ml of fluid and about 1 g of composition, the formulation has a complex modulus plateau between about 10^{5.1} Pa and about 10^{5.2} Pa. Clause 1304. The composition of any one of clauses 1101 to 1219, wherein when formulated in a formulation comprising about 1 ml of fluid and about 1 g of composition, the formulation has a complex modulus plateau between about 10^{5.2} Pa and about 10^{5.3} Pa. Clause 1305. The composition of any one of clauses 1101 to 1219, wherein when formulated in a formulation comprising about 1 ml of fluid and about 1 g of composition, the formulation has a complex modulus plateau between about 10^{5.3} Pa and about 10^{5.4} Pa. Clause 1306. The composition of any one of clauses 1101 to 1219, wherein when formulated in a formulation comprising about 1 ml of fluid and about 1 g of composition, the formulation has a complex modulus plateau between about 10^{5.4} Pa and about 10^{5.5} Pa. Clause 1307. The composition of any one of clauses 1101 to 1219, wherein when formulated in a formulation comprising about 1 ml of fluid and about 1 g of composition, the formulation has a complex modulus plateau between about 10^{5.5} Pa and about 10^{5.6} Pa. Clause 1308. The composition of any one of clauses 1101 to 1219, wherein when formulated in a formulation comprising about 1 ml of fluid and about 1 g of composition, the formulation has a complex modulus plateau between about 10^{5.6} Pa and about 10^{5.7} Pa. Clause 1309. The composition of any one of clauses 1101 to 1219, wherein when formulated in a formulation comprising about 1 ml of fluid and about 1 g of composition, the formulation has a complex modulus plateau between about 10^{5.7} Pa and about 10^{5.8} Pa. Clause 1310. The composition of any one of clauses 1101 to 1219, wherein when formulated in a formulation comprising about 1 ml of fluid and about 1 g of composition, the formulation has a complex modulus plateau between about 10^{5.8} Pa and about 10^{5.9} Pa. Clause 1311. The composition of any one of clauses 1101 to 1219, wherein when formulated in a formulation comprising about 1 ml of fluid and about 1 g of composition, the formulation has a complex modulus plateau between about 10^{5.9} Pa and about 10⁶ Pa. Clause 1312. The composition of any one of clauses 1101 to 1219, wherein when formulated in a formulation comprising about 1 ml of fluid and about 1 g of composition, the formulation has a complex modulus plateau between about 10⁶ Pa and about 10^{6.1} Pa. Clause 1313. The composition of any one of clauses 1101 to 1219, wherein when formulated in a formulation comprising about 1 ml of fluid and about 1 g of composition, the formulation has a complex modulus plateau between about 10^{6.1} Pa and about 10^{6.2} Pa. Clause 1314. The composition of any one of clauses 1101 to 1219, wherein when formulated in a formulation comprising about 1 ml of fluid and about 1 g of composition, the formulation has a complex modulus plateau between about 10^{6.2} Pa and about 10^{6.3} Pa. Clause 1315. The composition of any one of clauses 1101 to 1219, wherein when formulated in a formulation comprising about 1 ml of fluid and about 1 g of composition, the formulation has a complex modulus plateau between about 10^{6.3} Pa and about 10^{6.4} Pa. Clause 1316. The composition of any one of clauses 1101 to 1219, wherein when formulated in a formulation comprising about 1 ml of fluid and about 1 g of composition, the formulation has a complex modulus plateau between about 10^{6.4} Pa and about 10^{6.5} Pa.

Clause 1401. The composition of any one of clauses 1101 to 1219, wherein when formulated in a formulation comprising about 3 ml of fluid and about 1 g of composition, the formulation has a complex modulus plateau between about 10^{4.5} Pa and about 10^{5.5} Pa. Clause 1402. The composition of any one of clauses 1101 to 1219, wherein when formulated in a formulation comprising about 3 ml of fluid and about 1 g of composition, the formulation has a complex modulus plateau between about 10^{4.5} Pa and about 10^{4.6} Pa. Clause 1403. The composition of any one of clauses 1101 to 1219, wherein when formulated in a formulation comprising about 3 ml of fluid and about 1 g of composition, the formulation has a complex modulus plateau between about 10^{4.6} Pa and about 10^{4.7} Pa. Clause 1404. The composition of any one of clauses 1101 to 1219, wherein when formulated in a formulation comprising about 3 ml of fluid and about 1 g of composition, the formulation has a complex modulus plateau between about 10^{4.7} Pa and about 10^{4.8} Pa. Clause 1405. The composition of any one of clauses 1101 to 1219, wherein when formulated in a formulation comprising about 3 ml of fluid and about 1 g of composition, the formulation has a complex modulus plateau between about 10^{4.8} Pa and about 10^{4.9} Pa. Clause 1406. The composition of any one of clauses 1101 to 1219, wherein when formulated in a formulation comprising about 3 ml of fluid and about 1 g of composition, the formulation has a complex modulus plateau between about 10^{4.9} Pa and about 10⁵ Pa. Clause 1407. The composition of any one of clauses 1101 to 1219, wherein when formulated in a formulation comprising about 3 ml of fluid and about 1 g of composition, the formulation has a complex modulus plateau between about 10⁵ Pa and about 10^{5.1} Pa. Clause 1408. The composition of any one of clauses 1101 to 1219, wherein when formulated in a formulation comprising about 3 ml of fluid and about 1 g of composition, the formulation has a complex modulus plateau between about 10^{5.1} Pa and about 10^{5.2} Pa. Clause 1409. The composition of any one of clauses 1101 to 1219, wherein when formulated in a formulation comprising about 3 ml of fluid and about 1 g of composition, the formulation has a complex modulus plateau between about 10^{5.2} Pa and about 10^{5.3} Pa. Clause 1410. The composition of any one of clauses 1101 to 1219, wherein when formulated in a formulation comprising about 3 ml of fluid and about 1 g of composition, the formulation has a complex modulus plateau between about 10^{5.3} Pa and about 10^{5.4} Pa. Clause 1411. The composition of any one of clauses 1101 to 1219, wherein when formulated in a formulation comprising about 3 ml of fluid and about 1 g of composition, the formulation has a complex modulus plateau between about 10^{5.4} Pa and about 10^{5.5} Pa.

Clause 1501. The composition of any one of clauses 1101 to 1219, wherein when formulated in a formulation comprising about 3 ml of fluid and about 1 g of composition, the formulation has a complex modulus plateau between about 10⁴ Pa and about 10⁵ Pa. Clause 1501. The composition of any one of clauses 1101 to 1219, wherein when formulated in a formulation comprising about 3 ml of fluid and about 1 g of composition, the formulation has a complex modulus plateau between about 10⁴ Pa and about 10^{4.1} Pa. Clause 1502. The composition of any one of clauses 1101 to 1219, wherein when formulated in a formulation comprising about 3 ml of fluid and about 1 g of composition, the formulation has a complex modulus plateau between about 10^{4.1} Pa and about 10^{4.2} Pa. Clause 1503. The composition of any one of clauses 1101 to 1219, wherein when formulated in a formulation comprising about 3 ml of fluid and about 1 g of composition, the formulation has a complex modulus plateau between about 10^{4.2} Pa and about 10^{4.3} Pa. Clause 1504. The composition of any one of clauses 1101 to 1219, wherein when formulated in a formulation comprising about 3 ml of fluid and about 1 g of composition, the formulation has a complex modulus plateau between about 10^{4.3} Pa and about 10^{4.4} Pa. Clause 1505. The composition of any one of clauses 1101 to 1219, wherein when formulated in a formulation comprising about 3 ml of fluid and about 1 g of composition, the formulation has a complex modulus plateau between about 10^{4.4} Pa and about 10^{4.5} Pa. Clause 1506. The composition of any one of clauses 1101 to 1219, wherein when formulated in a formulation comprising about 3 ml of fluid and about 1 g of composition, the formulation has a complex modulus plateau between about 10^{4.5} Pa and about 10^{4.6} Pa. Clause 1507. The composition of any one of clauses 1101 to 1219, wherein when formulated in a formulation comprising about 3 ml of fluid and about 1 g of composition, the formulation has a complex modulus plateau between about 10^{4.6} Pa and about 10^{4.7} Pa. Clause 1508. The composition of any one of clauses 1101 to 1219, wherein when formulated in a formulation comprising about 3 ml of fluid and about 1 g of composition, the formulation has a complex modulus plateau between about 10^{4.7} Pa and about 10^{4.8} Pa. Clause 1509. The composition of any one of clauses 1101 to 1219, wherein when formulated in a formulation comprising about 3 ml of fluid and about 1 g of composition, the formulation has a complex modulus plateau between about 10^{4.8} Pa and about 10^{4.9} Pa. Clause 1510. The composition of any one of clauses 1101 to 1219, wherein when formulated in a formulation comprising about 3 ml of fluid and about 1 g of composition, the formulation has a complex modulus plateau between about 10^{4.9} Pa and about 10⁵ Pa.

Clause 1601. The composition of any one of clauses 1101 to 1219, wherein when formulated in a formulation comprising about 5 ml of fluid and about 1 g of composition, the formulation has a complex modulus plateau between about 10^{3.5} Pa and about 10⁵ Pa. Clause 1601. The composition of any one of clauses 1101 to 1219, wherein when formulated in a formulation comprising about 5 ml of fluid and about 1 g of composition, the formulation has a complex modulus plateau between about 10^{3.5} Pa and about 10^{3.6} Pa. Clause 1602. The composition of any one of clauses 1101 to 1219, wherein when formulated in a formulation comprising about 5 ml of fluid and about 1 g of composition, the formulation has a complex modulus plateau between about 10^{3.6} Pa and about 10^{3.7} Pa. Clause 1603. The composition of any one of clauses 1101 to 1219, wherein when formulated in a formulation comprising about 5 ml of fluid and about 1 g of composition, the formulation has a complex modulus plateau between about 10^{3.7} Pa and about 10^{3.8} Pa. Clause 1604. The composition of any one of clauses 1101 to 1219, wherein when formulated in a formulation comprising about 5 ml of fluid and about 1 g of composition, the formulation has a complex modulus plateau between about 10^{3.8} Pa and about 10^{3.9} Pa. Clause 1605. The composition of any one of clauses 1101 to 1219, wherein when formulated in a formulation comprising about 5 ml of fluid and about 1 g of composition, the formulation has a complex modulus plateau between about 10^{3.9} Pa and about 10⁴ Pa. Clause 1606. The composition of any one of clauses 1101 to 1219, wherein when formulated in a formulation comprising about 5 ml of fluid and about 1 g of composition, the formulation has a complex modulus plateau between about 10⁴ Pa and about 10^{4.1} Pa. Clause 1607. The composition of any one of clauses 1101 to 1219, wherein when formulated in a formulation comprising about 5 ml of fluid and about 1 g of composition, the formulation has a complex modulus plateau between about 10^{4.1} Pa and about 10^{4.2} Pa. Clause 1608. The composition of any one of clauses 1101 to 1219, wherein when formulated in a formulation comprising about 5 ml of fluid and about 1 g of composition, the formulation has a complex modulus plateau between about 10^{4.2} Pa and about 10^{4.3} Pa. Clause 1609. The composition of any one of clauses 1101 to 1219, wherein when formulated in a formulation comprising about 5 ml of fluid and about 1 g of composition, the formulation has a complex modulus plateau between about 10^{4.3} Pa and about 10^{4.4} Pa. Clause 1610. The composition of any one of clauses 1101 to 1219, wherein when formulated in a formulation comprising about 5 ml of fluid and about 1 g of composition, the formulation has a complex modulus plateau between about 10^{4.4} Pa and about 10^{4.5} Pa. Clause 1611. The composition of any one of clauses 1101 to 1219, wherein when formulated in a formulation comprising about 5 ml of fluid and about 1 g of composition, the formulation has a complex modulus plateau between about 10^{4.5} Pa and about 10^{4.6} Pa. Clause 1612. The composition of any one of clauses 1101 to 1219, wherein when formulated in a formulation comprising about 5 ml of fluid and about 1 g of composition, the formulation has a complex modulus plateau between about 10^{4.6} Pa and about 10^{4.7} Pa. Clause 1613. The composition of any one of clauses 1101 to 1219, wherein when formulated in a formulation comprising about 5 ml of fluid and about 1 g of composition, the formulation has a complex modulus plateau between about 10^{4.7} Pa and about 10^{4.8} Pa. Clause 1614. The composition of any one of clauses 1101 to 1219, wherein when formulated in a formulation comprising about 5 ml of fluid and about 1 g of composition, the formulation has a complex modulus plateau between about 10^{4.8} Pa and about 10^{4.9} Pa. Clause 1615. The composition of any one of clauses 1101 to 1219, wherein when formulated in a formulation comprising about 5 ml of fluid and about 1 g of composition, the formulation has a complex modulus plateau between about 10^{4.9} Pa and about 10⁵ Pa.

Clause 1701. The composition of any one of clauses 1101 to 1219, wherein when formulated in a formulation comprising between about 3 ml and about 5 ml of fluid and about 1 g of composition, the formulation has a complex modulus plateau between about 10^{3.5} Pa and about 10^{5.5} Pa. Clause 1702. The composition of any one of clauses 1101 to 1219, wherein when formulated in a formulation comprising between about 3 ml and about 5 ml of fluid and about 1 g of composition, the formulation has a complex modulus plateau between about 10^{3.5} Pa and about 10^{3.6} Pa. Clause 1703. The composition of any one of clauses 1101 to 1219, wherein when formulated in a formulation comprising between about 3 ml and about 5 ml of fluid and about 1 g of composition, the formulation has a complex modulus plateau between about 10^{3.6} Pa and about 10^{3.7} Pa. Clause 1704. The composition of any one of clauses 1101 to 1219, wherein when formulated in a formulation comprising between about 3 ml and about 5 ml of fluid and about 1 g of composition, the formulation has a complex modulus plateau between about 10^{3.7} Pa and about 10^{3.8} Pa. Clause 1705. The composition of any one of clauses 1101 to 1219, wherein when formulated in a formulation comprising between about 3 ml and about 5 ml of fluid and about 1 g of composition, the formulation has a complex modulus plateau between about 10^{3.8} Pa and about 10^{3.9} Pa. Clause 1706. The composition of any one of clauses 1101 to 1219, wherein when formulated in a formulation comprising between about 3 ml and about 5 ml of fluid and about 1 g of composition, the formulation has a complex modulus plateau between about 10^{3.9} Pa and about 10⁴ Pa. Clause 1707. The composition of any one of clauses 1101 to 1219, wherein when formulated in a formulation comprising between about 3 ml and about 5 ml of fluid and about 1 g of composition, the formulation has a complex modulus plateau between about 10⁴ Pa and about 10^{4.1} Pa. Clause 1708. The composition of any one of clauses 1101 to 1219, wherein when formulated in a formulation comprising between about 3 ml and about 5 ml of fluid and about 1 g of composition, the formulation has a complex modulus plateau between about 10^{4.1} Pa and about 10^{4.2} Pa. Clause 1709. The composition of any one of clauses 1101 to 1219, wherein when formulated in a formulation comprising between about 3 ml and about 5 ml of fluid and about 1 g of composition, the formulation has a complex modulus plateau between about 10^{4.2} Pa and about 10^{4.3} Pa. Clause 1710. The composition of any one of clauses 1101 to 1219, wherein when formulated in a formulation comprising between about 3 ml and about 5 ml of fluid and about 1 g of composition, the formulation has a complex modulus plateau between about 10^{4.3} Pa and about 10^{4.4} Pa. Clause 1711. The composition of any one of clauses 1101 to 1219, wherein when formulated in a formulation comprising between about 3 ml and about 5 ml of fluid and about 1 g of composition, the formulation has a complex modulus plateau between about 10^{4.4} Pa and about 10^{4.5} Pa. Clause 1712. The composition of any one of clauses 1101 to 1219, wherein when formulated in a formulation comprising between about 3 ml and about 5 ml of fluid and about 1 g of composition, the formulation has a complex modulus plateau between about 10^{4.5} Pa and about 10^{4.6} Pa. Clause 1713. The composition of any one of clauses 1101 to 1219, wherein when formulated in a formulation comprising between about 3 ml and about 5 ml of fluid and about 1 g of composition, the formulation has a complex modulus plateau between about 10^{4.6} Pa and about 10^{4.7} Pa. Clause 1714. The composition of any one of clauses 1101 to 1219, wherein when formulated in a formulation comprising between about 3 ml and about 5 ml of fluid and about 1 g of composition, the formulation has a complex modulus plateau between about 10^{4.7} Pa and about 10^{4.8} Pa. Clause 1715. The composition of any one of clauses 1101 to 1219, wherein when formulated in a formulation comprising between about 3 ml and about 5 ml of fluid and about 1 g of composition, the formulation has a complex modulus plateau between about 10^{4.8} Pa and about 10^{4.9} Pa. Clause 1716. The composition of any one of clauses 1101 to 1219, wherein when formulated in a formulation comprising between about 3 ml and about 5 ml of fluid and about 1 g of composition, the formulation has a complex modulus plateau between about 10^{4.9} Pa and about 10⁵ Pa. Clause 1717. The composition of any one of clauses 1101 to 1219, wherein when formulated in a formulation comprising between about 3 ml and about 5 ml of fluid and about 1 g of composition, the formulation has a complex modulus plateau between about 10⁵ Pa and about 10^{5.1} Pa. Clause 1718. The composition of any one of clauses 1101 to 1219, wherein when formulated in a formulation comprising between about 3 ml and about 5 ml of fluid and about 1 g of composition, the formulation has a complex modulus plateau between about 10^{5.1} Pa and about 10^{5.2} Pa. Clause 1719. The composition of any one of clauses 1101 to 1219, wherein when formulated in a formulation comprising between about 3 ml and about 5 ml of fluid and about 1 g of composition, the formulation has a complex modulus plateau between about 10^{5.2} Pa and about 10^{5.3} Pa. Clause 1720. The composition of any one of clauses 1101 to 1219, wherein when formulated in a formulation comprising between about 3 ml and about 5 ml of fluid and about 1 g of composition, the formulation has a complex modulus plateau between about 10^{5.3} Pa and about 10^{5.4} Pa. Clause 1721. The composition of any one of clauses 1101 to 1219, wherein when formulated in a formulation comprising between about 3 ml and about 5 ml of fluid and about 1 g of composition, the formulation has a complex modulus plateau between about 10^{5.4} Pa and about 10^{5.5} Pa.

Clause 1801. The composition of any one of clauses 1101 to 1219, wherein when formulated in a formulation comprising about 7 ml of fluid and about 1 g of composition, the formulation has a complex modulus plateau between about 10³ Pa and about 10^{4.5} Pa. Clause 1802. The composition of any one of clauses 1101 to 1219, wherein when formulated in a formulation comprising about 7 ml of fluid and about 1 g of composition, the formulation has a complex modulus plateau between about 10³ Pa and about 10^{3.1} Pa. Clause 1803. The composition of any one of clauses 1101 to 1219, wherein when formulated in a formulation comprising about 7 ml of fluid and about 1 g of composition, the formulation has a complex modulus plateau between about 10^{3.1} Pa and about 10^{3.2} Pa. Clause 1804. The composition of any one of clauses 1101 to 1219, wherein when formulated in a formulation comprising about 7 ml of fluid and about 1 g of composition, the formulation has a complex modulus plateau between about 10^{3.2} Pa and about 10^{3.3} Pa. Clause 1805. The composition of any one of clauses 1101 to 1219, wherein when formulated in a formulation comprising about 7 ml of fluid and about 1 g of composition, the formulation has a complex modulus plateau between about 10^{3.3} Pa and about 10^{3.4} Pa. Clause 1806. The composition of any one of clauses 1101 to 1219, wherein when formulated in a formulation comprising about 7 ml of fluid and about 1 g of composition, the formulation has a complex modulus plateau between about 10^{3.4} Pa and about 10^{3.5} Pa. Clause 1807. The composition of any one of clauses 1101 to 1219, wherein when formulated in a formulation comprising about 7 ml of fluid and about 1 g of composition, the formulation has a complex modulus plateau between about 10^{3.5} Pa and about 10^{3.6} Pa. Clause 1808. The composition of any one of clauses 1101 to 1219, wherein when formulated in a formulation comprising about 7 ml of fluid and about 1 g of composition, the formulation has a complex modulus plateau between about 10^{3.6} Pa and about 10^{3.7} Pa. Clause 1809. The composition of any one of clauses 1101 to 1219, wherein when formulated in a formulation comprising about 7 ml of fluid and about 1 g of composition, the formulation has a complex modulus plateau between about 10^{3.7} Pa and about 10^{3.8} Pa. Clause 1810. The composition of any one of clauses 1101 to 1219, wherein when formulated in a formulation comprising about 7 ml of fluid and about 1 g of composition, the formulation has a complex modulus plateau between about 10^{3.8} Pa and about 10^{3.9} Pa. Clause 1811. The composition of any one of clauses 1101 to 1219, wherein when formulated in a formulation comprising about 7 ml of fluid and about 1 g of composition, the formulation has a complex modulus plateau between about 10^{3.9} Pa and about 10⁴ Pa. Clause 1812. The composition of any one of clauses 1101 to 1219, wherein when formulated in a formulation comprising about 7 ml of fluid and about 1 g of composition, the formulation has a complex modulus plateau between about 10⁴ Pa and about 10^{4.1} Pa. Clause 1813. The composition of any one of clauses 1101 to 1219, wherein when formulated in a formulation comprising about 7 ml of fluid and about 1 g of composition, the formulation has a complex modulus plateau between about 10^{4.1} Pa and about 10^{4.2} Pa. Clause 1814. The composition of any one of clauses 1101 to 1219, wherein when formulated in a formulation comprising about 7 ml of fluid and about 1 g of composition, the formulation has a complex modulus plateau between about 10^{4.2} Pa and about 10^{4.3} Pa. Clause 1815. The composition of any one of clauses 1101 to 1219, wherein when formulated in a formulation comprising about 7 ml of fluid and about 1 g of composition, the formulation has a complex modulus plateau between about 10^{4.3} Pa and about 10^{4.4} Pa. Clause 1816. The composition of any one of clauses 1101 to 1219, wherein when formulated in a formulation comprising about 7 ml of fluid and about 1 g of composition, the formulation has a complex modulus plateau between about 10^{4.4} Pa and about 10^{4.5} Pa.

Clause 1901. The composition of any one of clauses 1101 to 1219, wherein when formulated in a formulation comprising between about 3 ml and about 7 ml of fluid and about 1 g of composition, the formulation has a complex modulus plateau between about 10³ Pa and about 10^{5.5} Pa. Clause 1902. The composition of any one of clauses 1101 to 1219, wherein when formulated in a formulation comprising between about 3 ml and about 7 ml of fluid and about 1 g of composition, the formulation has a complex modulus plateau between about 10³ Pa and about 10^{3.1} Pa. Clause 1903. The composition of any one of clauses 1101 to 1219, wherein when formulated in a formulation comprising between about 3 ml and about 7 ml of fluid and about 1 g of composition, the formulation has a complex modulus plateau between about 10^{3.1} Pa and about 10^{3.2} Pa. Clause 1904. The composition of any one of clauses 1101 to 1219, wherein when formulated in a formulation comprising between about 3 ml and about 7 ml of fluid and about 1 g of composition, the formulation has a complex modulus plateau between about 10^{3.2} Pa and about 10^{3.3} Pa. Clause 1905. The composition of any one of clauses 1101 to 1219, wherein when formulated in a formulation comprising between about 3 ml and about 7 ml of fluid and about 1 g of composition, the formulation has a complex modulus plateau between about 10^{3.3} Pa and about 10^{3.4} Pa. Clause 1906. The composition of any one of clauses 1101 to 1219, wherein when formulated in a formulation comprising between about 3 ml and about 7 ml of fluid and about 1 g of composition, the formulation has a complex modulus plateau between about 10^{3.4} Pa and about 10^{3.5} Pa. Clause 1907. The composition of any one of clauses 1101 to 1219, wherein when formulated in a formulation comprising between about 3 ml and about 7 ml of fluid and about 1 g of composition, the formulation has a complex modulus plateau between about 10^{3.5} Pa and about 10^{3.6} Pa. Clause 1908. The composition of any one of clauses 1101 to 1219, wherein when formulated in a formulation comprising between about 3 ml and about 7 ml of fluid and about 1 g of composition, the formulation has a complex modulus plateau between about 10^{3.6} Pa and about 10^{3.7} Pa. Clause 1909. The composition of any one of clauses 1101 to 1219, wherein when formulated in a formulation comprising between about 3 ml and about 7 ml of fluid and about 1 g of composition, the formulation has a complex modulus plateau between about 10^{3.7} Pa and about 10^{3.8} Pa. Clause 1910. The composition of any one of clauses 1101 to 1219, wherein when formulated in a formulation comprising between about 3 ml and about 7 ml of fluid and about 1 g of composition, the formulation has a complex modulus plateau between about 10^{3.8} Pa and about 10^{3.9} Pa. Clause 1911. The composition of any one of clauses 1101 to 1219, wherein when formulated in a formulation comprising between about 3 ml and about 7 ml of fluid and about 1 g of composition, the formulation has a complex modulus plateau between about 10^{3.9} Pa and about 10⁴ Pa. Clause 1912. The composition of any one of clauses 1101 to 1219, wherein when formulated in a formulation comprising between about 3 ml and about 7 ml of fluid and about 1 g of composition, the formulation has a complex modulus plateau between about 10⁴ Pa and about 10^{4.1} Pa. Clause 1913. The composition of any one of clauses 1101 to 1219, wherein when formulated in a formulation comprising between about 3 ml and about 7 ml of fluid and about 1 g of composition, the formulation has a complex modulus plateau between about 10^{4.1} Pa and about 10^{4.2} Pa. Clause 1914. The composition of any one of clauses 1101 to 1219, wherein when formulated in a formulation comprising between about 3 ml and about 7 ml of fluid and about 1 g of composition, the formulation has a complex modulus plateau between about 10^{4.2} Pa and about 10^{4.3} Pa. Clause 1915. The composition of any one of clauses 1101 to 1219, wherein when formulated in a formulation comprising between about 3 ml and about 7 ml of fluid and about 1 g of composition, the formulation has a complex modulus plateau between about 10^{4.3} Pa and about 10^{4.4} Pa. Clause 1916. The composition of any one of clauses 1101 to 1219, wherein when formulated in a formulation comprising between about 3 ml and about 7 ml of fluid and about 1 g of composition, the formulation has a complex modulus plateau between about 10^{4.4} Pa and about 10^{4.5} Pa. Clause 1917. The composition of any one of clauses 1101 to 1219, wherein when formulated in a formulation comprising between about 3 ml and about 7 ml of fluid and about 1 g of composition, the formulation has a complex modulus plateau between about 10^{4.5} Pa and about 10^{4.6} Pa. Clause 1918. The composition of any one of clauses 1101 to 1219, wherein when formulated in a formulation comprising between about 3 ml and about 7 ml of fluid and about 1 g of composition, the formulation has a complex modulus plateau between about 10^{4.6} Pa and about 10^{4.7} Pa. Clause 1919. The composition of any one of clauses 1101 to 1219, wherein when formulated in a formulation comprising between about 3 ml and about 7 ml of fluid and about 1 g of composition, the formulation has a complex modulus plateau between about 10^{4.7} Pa and about 10^{4.8} Pa. Clause 1920. The composition of any one of clauses 1101 to 1219, wherein when formulated in a formulation comprising between about 3 ml and about 7 ml of fluid and about 1 g of composition, the formulation has a complex modulus plateau between about 10^{4.8} Pa and about 10^{4.9} Pa. Clause 1921. The composition of any one of clauses 1101 to 1219, wherein when formulated in a formulation comprising between about 3 ml and about 7 ml of fluid and about 1 g of composition, the formulation has a complex modulus plateau between about 10^{4.9} Pa and about 10⁵ Pa. Clause 1922. The composition of any one of clauses 1101 to 1219, wherein when formulated in a formulation comprising between about 3 ml and about 7 ml of fluid and about 1 g of composition, the formulation has a complex modulus plateau between about 10⁵ Pa and about 10^{5.1} Pa. Clause 1923. The composition of any one of clauses 1101 to 1219, wherein when formulated in a formulation comprising between about 3 ml and about 7 ml of fluid and about 1 g of composition, the formulation has a complex modulus plateau between about 10^{5.1} Pa and about 10^{5.2} Pa. Clause 1924. The composition of any one of clauses 1101 to 1219, wherein when formulated in a formulation comprising between about 3 ml and about 7 ml of fluid and about 1 g of composition, the formulation has a complex modulus plateau between about 10^{5.2} Pa and about 10^{5.3} Pa. Clause 1925. The composition of any one of clauses 1101 to 1219, wherein when formulated in a formulation comprising between about 3 ml and about 7 ml of fluid and about 1 g of composition, the formulation has a complex modulus plateau between about 10^{5.3} Pa and about 10^{5.4} Pa. Clause 1926. The composition of any one of clauses 1101 to 1219, wherein when formulated in a formulation comprising between about 3 ml and about 7 ml of fluid and about 1 g of composition, the formulation has a complex modulus plateau between about 10^{5.4} Pa and about 10^{5.5} Pa.

Clause 2100. The composition of any one of clauses 1101 to 1219, wherein when formulated in a formulation comprising between about 1 ml and about 7 ml of fluid and about 1 g of composition, the formulation has a complex modulus plateau between about 10³ Pa and about 10^{6.5} Pa. Clause 2101. The composition of any one of clauses 1101 to 1219, wherein when formulated in a formulation comprising between about 1 ml and about 7 ml of fluid and about 1 g of composition, the formulation has a complex modulus plateau between about 10³ Pa and about 10^{3.1} Pa. Clause 2102. The composition of any one of clauses 1101 to 1219, wherein when formulated in a formulation comprising between about 1 ml and about 7 ml of fluid and about 1 g of composition, the formulation has a complex modulus plateau between about 10^{3.1} Pa and about 10^{3.2} Pa. Clause 2103. The composition of any one of clauses 1101 to 1219, wherein when formulated in a formulation comprising between about 1 ml and about 7 ml of fluid and about 1 g of composition, the formulation has a complex modulus plateau between about 10^{3.2} Pa and about 10^{3.3} Pa. Clause 2104. The composition of any one of clauses 1101 to 1219, wherein when formulated in a formulation comprising between about 1 ml and about 7 ml of fluid and about 1 g of composition, the formulation has a complex modulus plateau between about 10^{3.3} Pa and about 10^{3.4} Pa. Clause 2105. The composition of any one of clauses 1101 to 1219, wherein when formulated in a formulation comprising between about 1 ml and about 7 ml of fluid and about 1 g of composition, the formulation has a complex modulus plateau between about 10^{3.4} Pa and about 10^{3.5} Pa. Clause 2106. The composition of any one of clauses 1101 to 1219, wherein when formulated in a formulation comprising between about 1 ml and about 7 ml of fluid and about 1 g of composition, the formulation has a complex modulus plateau between about 10^{3.5} Pa and about 10^{3.6} Pa. Clause 2107. The composition of any one of clauses 1101 to 1219, wherein when formulated in a formulation comprising between about 1 ml and about 7 ml of fluid and about 1 g of composition, the formulation has a complex modulus plateau between about 10^{3.6} Pa and about 10^{3.7} Pa. Clause 2108. The composition of any one of clauses 1101 to 1219, wherein when formulated in a formulation comprising between about 1 ml and about 7 ml of fluid and about 1 g of composition, the formulation has a complex modulus plateau between about 10^{3.7} Pa and about 10^{3.8} Pa. Clause 2109. The composition of any one of clauses 1101 to 1219, wherein when formulated in a formulation comprising between about 1 ml and about 7 ml of fluid and about 1 g of composition, the formulation has a complex modulus plateau between about 10^{3.8} Pa and about 10^{3.9} Pa. Clause 2110. The composition of any one of clauses 1101 to 1219, wherein when formulated in a formulation comprising between about 1 ml and about 7 ml of fluid and about 1 g of composition, the formulation has a complex modulus plateau between about 10^{3.9} Pa and about 10⁴ Pa. Clause 2111. The composition of any one of clauses 1101 to 1219, wherein when formulated in a formulation comprising between about 1 ml and about 7 ml of fluid and about 1 g of composition, the formulation has a complex modulus plateau between about 10⁴ Pa and about 10^{4.1} Pa. Clause 2112. The composition of any one of clauses 1101 to 1219, wherein when formulated in a formulation comprising between about 1 ml and about 7 ml of fluid and about 1 g of composition, the formulation has a complex modulus plateau between about 10^{4.1} Pa and about 10^{4.2} Pa. Clause 2113. The composition of any one of clauses 1101 to 1219, wherein when formulated in a formulation comprising between about 1 ml and about 7 ml of fluid and about 1 g of composition, the formulation has a complex modulus plateau between about 10^{4.2} Pa and about 10^{4.3} Pa. Clause 2114. The composition of any one of clauses 1101 to 1219, wherein when formulated in a formulation comprising between about 1 ml and about 7 ml of fluid and about 1 g of composition, the formulation has a complex modulus plateau between about 10^{4.3} Pa and about 10^{4.4} Pa. Clause 2115. The composition of any one of clauses 1101 to 1219, wherein when formulated in a formulation comprising between about 1 ml and about 7 ml of fluid and about 1 g of composition, the formulation has a complex modulus plateau between about 10^{4.4} Pa and about 10^{4.5} Pa. Clause 2116. The composition of any one of clauses 1101 to 1219, wherein when formulated in a formulation comprising between about 1 ml and about 7 ml of fluid and about 1 g of composition, the formulation has a complex modulus plateau between about 10^{4.5} Pa and about 10^{4.6} Pa. Clause 2117. The composition of any one of clauses 1101 to 1219, wherein when formulated in a formulation comprising between about 1 ml and about 7 ml of fluid and about 1 g of composition, the formulation has a complex modulus plateau between about 10^{4.6} Pa and about 10^{4.7} Pa. Clause 2118. The composition of any one of clauses 1101 to 1219, wherein when formulated in a formulation comprising between about 1 ml and about 7 ml of fluid and about 1 g of composition, the formulation has a complex modulus plateau between about 10^{4.7} Pa and about 10^{4.8} Pa. Clause 2119. The composition of any one of clauses 1101 to 1219, wherein when formulated in a formulation comprising between about 1 ml and about 7 ml of fluid and about 1 g of composition, the formulation has a complex modulus plateau between about 10^{4.8} Pa and about 10^{4.9} Pa. Clause 2120. The composition of any one of clauses 1101 to 1219, wherein when formulated in a formulation comprising between about 1 ml and about 7 ml of fluid and about 1 g of composition, the formulation has a complex modulus plateau between about 10^{4.9} Pa and about 10⁵ Pa. Clause 2121. The composition of any one of clauses 1101 to 1219, wherein when formulated in a formulation comprising between about 1 ml and about 7 ml of fluid and about 1 g of composition, the formulation has a complex modulus plateau between about 10⁵ Pa and about 10^{5.1} Pa. Clause 2122. The composition of any one of clauses 1101 to 1219, wherein when formulated in a formulation comprising between about 1 ml and about 7 ml of fluid and about 1 g of composition, the formulation has a complex modulus plateau between about 10^{5.1} Pa and about 10^{5.2} Pa. Clause 2123. The composition of any one of clauses 1101 to 1219, wherein when formulated in a formulation comprising between about 1 ml and about 7 ml of fluid and about 1 g of composition, the formulation has a complex modulus plateau between about 10^{5.2} Pa and about 10^{5.3} Pa. Clause 2124. The composition of any one of clauses 1101 to 1219, wherein when formulated in a formulation comprising between about 1 ml and about 7 ml of fluid and about 1 g of composition, the formulation has a complex modulus plateau between about 10^{5.3} Pa and about 10^{5.4} Pa. Clause 2125. The composition of any one of clauses 1101 to 1219, wherein when formulated in a formulation comprising between about 1 ml and about 7 ml of fluid and about 1 g of composition, the formulation has a complex modulus plateau between about 10^{5.4} Pa and about 10^{5.5} Pa. Clause 2126. The composition of any one of clauses 1101 to 1219, wherein when formulated in a formulation comprising between about 1 ml and about 7 ml of fluid and about 1 g of composition, the formulation has a complex modulus plateau between about 10^{5.5} Pa and about 10^{5.6} Pa. Clause 2127. The composition of any one of clauses 1101 to 1219, wherein when formulated in a formulation comprising between about 1 ml and about 7 ml of fluid and about 1 g of composition, the formulation has a complex modulus plateau between about 10^{5.6} Pa and about 10^{5.7} Pa. Clause 2128. The composition of any one of clauses 1101 to 1219, wherein when formulated in a formulation comprising between about 1 ml and about 7 ml of fluid and about 1 g of composition, the formulation has a complex modulus plateau between about 10^{5.7} Pa and about 10^{5.8} Pa. Clause 2129. The composition of any one of clauses 1101 to 1219, wherein when formulated in a formulation comprising between about 1 ml and about 7 ml of fluid and about 1 g of composition, the formulation has a complex modulus plateau between about 10^{5.8} Pa and about 10^{5.9} Pa. Clause 2130. The composition of any one of clauses 1101 to 1219, wherein when formulated in a formulation comprising between about 1 ml and about 7 ml of fluid and about 1 g of composition, the formulation has a complex modulus plateau between about 10^{5.9} Pa and about 10⁶ Pa. Clause 2131. The composition of any one of clauses 1101 to 1219, wherein when formulated in a formulation comprising between about 1 ml and about 7 ml of fluid and about 1 g of composition, the formulation has a complex modulus plateau between about 10⁶ Pa and about 10^{6.1} Pa. Clause 2132. The composition of any one of clauses 1101 to 1219, wherein when formulated in a formulation comprising between about 1 ml and about 7 ml of fluid and about 1 g of composition, the formulation has a complex modulus plateau between about 10^{6.1} Pa and about 10^{6.2} Pa. Clause 2133. The composition of any one of clauses 1101 to 1219, wherein when formulated in a formulation comprising between about 1 ml and about 7 ml of fluid and about 1 g of composition, the formulation has a complex modulus plateau between about 10^{6.2} Pa and about 10^{6.3} Pa. Clause 2134. The composition of any one of clauses 1101 to 1219, wherein when formulated in a formulation comprising between about 1 ml and about 7 ml of fluid and about 1 g of composition, the formulation has a complex modulus plateau between about 10^{6.3} Pa and about 10^{6.4} Pa. Clause 2135. The composition of any one of clauses 1101 to 1219, wherein when formulated in a formulation comprising between about 1 ml and about 7 ml of fluid and about 1 g of composition, the formulation has a complex modulus plateau between about 10^{6.4} Pa and about 10^{6.5} Pa.

Clause 2200. The composition of any one of clauses 1101 to 1219, wherein when formulated in a formulation comprising about 1 ml of fluid and about 1 g of composition, the formulation has a storage modulus between about 10⁵ Pa and about 10^{6.5} Pa at an angular frequency between about 10⁻¹ rad/s and about 10² rad/s. Clause 2201. The composition of any one of clauses 1101 to 1219, wherein when formulated in a formulation comprising about 1 ml of fluid and about 1 g of composition, the formulation has a storage modulus between about 10⁵ Pa and about 10^{5.1} Pa at an angular frequency between about 10⁻¹ rad/s and about 10² rad/s. Clause 2202. The composition of any one of clauses 1101 to 1219, wherein when formulated in a formulation comprising about 1 ml of fluid and about 1 g of composition, the formulation has a storage modulus between about 10^{5.1} Pa and about 10^{5.2} Pa at an angular frequency between about 10⁻¹ rad/s and about 10² rad/s. Clause 2203. The composition of any one of clauses 1101 to 1219, wherein when formulated in a formulation comprising about 1 ml of fluid and about 1 g of composition, the formulation has a storage modulus between about 10^{5.2} Pa and about 10^{5.3} Pa at an angular frequency between about 10⁻¹ rad/s and about 10² rad/s. Clause 2204. The composition of any one of clauses 1101 to 1219, wherein when formulated in a formulation comprising about 1 ml of fluid and about 1 g of composition, the formulation has a storage modulus between about 10^{5.3} Pa and about 10^{5.4} Pa at an angular frequency between about 10⁻¹ rad/s and about 10² rad/s. Clause 2205. The composition of any one of clauses 1101 to 1219, wherein when formulated in a formulation comprising about 1 ml of fluid and about 1 g of composition, the formulation has a storage modulus between about 10^{5.4} Pa and about 10^{5.5} Pa at an angular frequency between about 10⁻¹ rad/s and about 10² rad/s. Clause 2206. The composition of any one of clauses 1101 to 1219, wherein when formulated in a formulation comprising about 1 ml of fluid and about 1 g of composition, the formulation has a storage modulus between about 10^{5.5} Pa and about 10^{5.6} Pa at an angular frequency between about 10⁻¹ rad/s and about 10² rad/s. Clause 2207. The composition of any one of clauses 1101 to 1219, wherein when formulated in a formulation comprising about 1 ml of fluid and about 1 g of composition, the formulation has a storage modulus between about 10^{5.6} Pa and about 10^{5.7} Pa at an angular frequency between about 10⁻¹ rad/s and about 10² rad/s. Clause 2208. The composition of any one of clauses 1101 to 1219, wherein when formulated in a formulation comprising about 1 ml of fluid and about 1 g of composition, the formulation has a storage modulus between about 10^{5.7} Pa and about 10^{5.8} Pa at an angular frequency between about 10⁻¹ rad/s and about 10² rad/s. Clause 2209. The composition of any one of clauses 1101 to 1219, wherein when formulated in a formulation comprising about 1 ml of fluid and about 1 g of composition, the formulation has a storage modulus between about 10^{5.8} Pa and about 10^{5.9} Pa at an angular frequency between about 10⁻¹ rad/s and about 10² rad/s. Clause 2210. The composition of any one of clauses 1101 to 1219, wherein when formulated in a formulation comprising about 1 ml of fluid and about 1 g of composition, the formulation has a storage modulus between about 10^{5.9} Pa and about 10⁶ Pa at an angular frequency between about 10⁻¹ rad/s and about 10² rad/s. Clause 2211. The composition of any one of clauses 1101 to 1219, wherein when formulated in a formulation comprising about 1 ml of fluid and about 1 g of composition, the formulation has a storage modulus between about 10⁶ Pa and about 10^{6.1} Pa at an angular frequency between about 10⁻¹ rad/s and about 10² rad/s. Clause 2212. The composition of any one of clauses 1101 to 1219, wherein when formulated in a formulation comprising about 1 ml of fluid and about 1 g of composition, the formulation has a storage modulus between about 10^{6.1} Pa and about 10^{6.2} Pa at an angular frequency between about 10⁻¹ rad/s and about 10² rad/s. Clause 2213. The composition of any one of clauses 1101 to 1219, wherein when formulated in a formulation comprising about 1 ml of fluid and about 1 g of composition, the formulation has a storage modulus between about 10^{6.2} Pa and about 10^{6.3} Pa at an angular frequency between about 10⁻¹ rad/s and about 10² rad/s. Clause 2214. The composition of any one of clauses 1101 to 1219, wherein when formulated in a formulation comprising about 1 ml of fluid and about 1 g of composition, the formulation has a storage modulus between about 10^{6.3} Pa and about 10^{6.4} Pa at an angular frequency between about 10⁻¹ rad/s and about 10² rad/s. Clause 2215. The composition of any one of clauses 1101 to 1219, wherein when formulated in a formulation comprising about 1 ml of fluid and about 1 g of composition, the formulation has a storage modulus between about 10^{6.4} Pa and about 10^{6.5} Pa at an angular frequency between about 10⁻¹ rad/s and about 10² rad/s.

Clause 2300. The composition of any one of clauses 1101 to 1219, wherein when formulated in a formulation comprising about 1 ml of fluid and about 1 g of composition, the formulation has a loss modulus between about 10^{4.5} Pa and about 10⁶ Pa at an angular frequency between about 10⁻¹ rad/s and about 10² rad/s. Clause 2301. The composition of any one of clauses 1101 to 1219, wherein when formulated in a formulation comprising about 1 ml of fluid and about 1 g of composition, the formulation has a loss modulus between about 10^{4.5} Pa and about 10^{4.6} Pa at an angular frequency between about 10⁻¹ rad/s and about 10² rad/s. Clause 2302. The composition of any one of clauses 1101 to 1219, wherein when formulated in a formulation comprising about 1 ml of fluid and about 1 g of composition, the formulation has a loss modulus between about 10^{4.6} Pa and about 10^{4.7} Pa at an angular frequency between about 10⁻¹ rad/s and about 10² rad/s. Clause 2303. The composition of any one of clauses 1101 to 1219, wherein when formulated in a formulation comprising about 1 ml of fluid and about 1 g of composition, the formulation has a loss modulus between about 10^{4.7} Pa and about 10^{4.8} Pa at an angular frequency between about 10⁻¹ rad/s and about 10² rad/s. Clause 2304. The composition of any one of clauses 1101 to 1219, wherein when formulated in a formulation comprising about 1 ml of fluid and about 1 g of composition, the formulation has a loss modulus between about 10^{4.8} Pa and about 10^{4.9} Pa at an angular frequency between about 10⁻¹ rad/s and about 10² rad/s. Clause 2305. The composition of any one of clauses 1101 to 1219, wherein when formulated in a formulation comprising about 1 ml of fluid and about 1 g of composition, the formulation has a loss modulus between about 10^{4.9} Pa and about 10⁵ Pa at an angular frequency between about 10⁻¹ rad/s and about 10² rad/s. Clause 2306. The composition of any one of clauses 1101 to 1219, wherein when formulated in a formulation comprising about 1 ml of fluid and about 1 g of composition, the formulation has a loss modulus between about 10⁵ Pa and about 10^{5.1} Pa at an angular frequency between about 10⁻¹ rad/s and about 10² rad/s. Clause 2307. The composition of any one of clauses 1101 to 1219, wherein when formulated in a formulation comprising about 1 ml of fluid and about 1 g of composition, the formulation has a loss modulus between about 10^{5.1} Pa and about 10^{5.2} Pa at an angular frequency between about 10⁻¹ rad/s and about 10² rad/s. Clause 2308. The composition of any one of clauses 1101 to 1219, wherein when formulated in a formulation comprising about 1 ml of fluid and about 1 g of composition, the formulation has a loss modulus between about 10^{5.2} Pa and about 10^{5.3} Pa at an angular frequency between about 10⁻¹ rad/s and about 10² rad/s. Clause 2309. The composition of any one of clauses 1101 to 1219, wherein when formulated in a formulation comprising about 1 ml of fluid and about 1 g of composition, the formulation has a loss modulus between about 10^{5.3} Pa and about 10^{5.4} Pa at an angular frequency between about 10⁻¹ rad/s and about 10² rad/s. Clause 2310. The composition of any one of clauses 1101 to 1219, wherein when formulated in a formulation comprising about 1 ml of fluid and about 1 g of composition, the formulation has a loss modulus between about 10^{5.4} Pa and about 10^{5.5} Pa at an angular frequency between about 10⁻¹ rad/s and about 10² rad/s. Clause 2311. The composition of any one of clauses 1101 to 1219, wherein when formulated in a formulation comprising about 1 ml of fluid and about 1 g of composition, the formulation has a loss modulus between about 10^{5.5} Pa and about 10^{5.6} Pa at an angular frequency between about 10⁻¹ rad/s and about 10² rad/s. Clause 2312. The composition of any one of clauses 1101 to 1219, wherein when formulated in a formulation comprising about 1 ml of fluid and about 1 g of composition, the formulation has a loss modulus between about 10^{5.6} Pa and about 10^{5.7} Pa at an angular frequency between about 10⁻¹ rad/s and about 10² rad/s. Clause 2313. The composition of any one of clauses 1101 to 1219, wherein when formulated in a formulation comprising about 1 ml of fluid and about 1 g of composition, the formulation has a loss modulus between about 10^{5.7} Pa and about 10^{5.8} Pa at an angular frequency between about 10⁻¹ rad/s and about 10² rad/s. Clause 2314. The composition of any one of clauses 1101 to 1219, wherein when formulated in a formulation comprising about 1 ml of fluid and about 1 g of composition, the formulation has a loss modulus between about 10^{5.8} Pa and about 10^{5.9} Pa at an angular frequency between about 10⁻¹ rad/s and about 10² rad/s. Clause 2315. The composition of any one of clauses 1101 to 1219, wherein when formulated in a formulation comprising about 1 ml of fluid and about 1 g of composition, the formulation has a loss modulus between about 10^{5.9} Pa and about 10⁶ Pa at an angular frequency between about 10⁻¹ rad/s and about 10² rad/s.

Clause 2400. The composition of any one of clauses 1101 to 1219, wherein when formulated in a formulation comprising about 3 ml of fluid and about 1 g of composition, the formulation has a storage modulus between about 10⁴ Pa and about 10^{5.5} Pa at an angular frequency between about 10⁻¹ rad/s and about 10² rad/s. Clause 2401. The composition of any one of clauses 1101 to 1219, wherein when formulated in a formulation comprising about 3 ml of fluid and about 1 g of composition, the formulation has a storage modulus between about 10⁴ Pa and about 10^{4.1} Pa at an angular frequency between about 10⁻¹ rad/s and about 10² rad/s. Clause 2402. The composition of any one of clauses 1101 to 1219, wherein when formulated in a formulation comprising about 3 ml of fluid and about 1 g of composition, the formulation has a storage modulus between about 10^{4.1} Pa and about 10^{4.2} Pa at an angular frequency between about 10⁻¹ rad/s and about 10² rad/s. Clause 2403. The composition of any one of clauses 1101 to 1219, wherein when formulated in a formulation comprising about 3 ml of fluid and about 1 g of composition, the formulation has a storage modulus between about 10^{4.2} Pa and about 10^{4.3} Pa at an angular frequency between about 10⁻¹ rad/s and about 10² rad/s. Clause 2404. The composition of any one of clauses 1101 to 1219, wherein when formulated in a formulation comprising about 3 ml of fluid and about 1 g of composition, the formulation has a storage modulus between about 10^{4.3} Pa and about 10^{4.4} Pa at an angular frequency between about 10⁻¹ rad/s and about 10² rad/s. Clause 2405. The composition of any one of clauses 1101 to 1219, wherein when formulated in a formulation comprising about 3 ml of fluid and about 1 g of composition, the formulation has a storage modulus between about 10^{4.4} Pa and about 10^{4.5} Pa at an angular frequency between about 10⁻¹ rad/s and about 10² rad/s. Clause 2406. The composition of any one of clauses 1101 to 1219, wherein when formulated in a formulation comprising about 3 ml of fluid and about 1 g of composition, the formulation has a storage modulus between about 10^{4.5} Pa and about 10^{4.6} Pa at an angular frequency between about 10⁻¹ rad/s and about 10² rad/s. Clause 2407. The composition of any one of clauses 1101 to 1219, wherein when formulated in a formulation comprising about 3 ml of fluid and about 1 g of composition, the formulation has a storage modulus between about 10^{4.6} Pa and about 10^{4.7} Pa at an angular frequency between about 10⁻¹ rad/s and about 10² rad/s. Clause 2408. The composition of any one of clauses 1101 to 1219, wherein when formulated in a formulation comprising about 3 ml of fluid and about 1 g of composition, the formulation has a storage modulus between about 10^{4.7} Pa and about 10^{4.8} Pa at an angular frequency between about 10⁻¹ rad/s and about 10² rad/s. Clause 2409. The composition of any one of clauses 1101 to 1219, wherein when formulated in a formulation comprising about 3 ml of fluid and about 1 g of composition, the formulation has a storage modulus between about 10^{4.8} Pa and about 10^{4.9} Pa at an angular frequency between about 10⁻¹ rad/s and about 10² rad/s. Clause 2410. The composition of any one of clauses 1101 to 1219, wherein when formulated in a formulation comprising about 3 ml of fluid and about 1 g of composition, the formulation has a storage modulus between about 10^{4.9} Pa and about 10⁵ Pa at an angular frequency between about 10⁻¹ rad/s and about 10² rad/s. Clause 2411. The composition of any one of clauses 1101 to 1219, wherein when formulated in a formulation comprising about 3 ml of fluid and about 1 g of composition, the formulation has a storage modulus between about 10⁵ Pa and about 10^{5.1} Pa at an angular frequency between about 10⁻¹ rad/s and about 10² rad/s. Clause 2412. The composition of any one of clauses 1101 to 1219, wherein when formulated in a formulation comprising about 3 ml of fluid and about 1 g of composition, the formulation has a storage modulus between about 10^{5.1} Pa and about 10^{5.2} Pa at an angular frequency between about 10⁻¹ rad/s and about 10² rad/s. Clause 2413. The composition of any one of clauses 1101 to 1219, wherein when formulated in a formulation comprising about 3 ml of fluid and about 1 g of composition, the formulation has a storage modulus between about 10^{5.2} Pa and about 10^{5.3} Pa at an angular frequency between about 10⁻¹ rad/s and about 10² rad/s. Clause 2414. The composition of any one of clauses 1101 to 1219, wherein when formulated in a formulation comprising about 3 ml of fluid and about 1 g of composition, the formulation has a storage modulus between about 10^{5.3} Pa and about 10^{5.4} Pa at an angular frequency between about 10⁻¹ rad/s and about 10² rad/s. Clause 2415. The composition of any one of clauses 1101 to 1219, wherein when formulated in a formulation comprising about 3 ml of fluid and about 1 g of composition, the formulation has a storage modulus between about 10^{5.4} Pa and about 10^{5.5} Pa at an angular frequency between about 10⁻¹ rad/s and about 10² rad/s.

Clause 2500. The composition of any one of clauses 1101 to 1219, wherein when formulated in a formulation comprising about 3 ml of fluid and about 1 g of composition, the formulation has a loss modulus between about 10^{3.5} Pa and about 10⁵ Pa at an angular frequency between about 10⁻¹ rad/s and about 10² rad/s. Clause 2501. The composition of any one of clauses 1101 to 1219, wherein when formulated in a formulation comprising about 3 ml of fluid and about 1 g of composition, the formulation has a loss modulus between about 10^{3.5} Pa and about 10^{3.6} Pa at an angular frequency between about 10⁻¹ rad/s and about 10² rad/s. Clause 2502. The composition of any one of clauses 1101 to 1219, wherein when formulated in a formulation comprising about 3 ml of fluid and about 1 g of composition, the formulation has a loss modulus between about 10^{3.6} Pa and about 10^{3.7} Pa at an angular frequency between about 10⁻¹ rad/s and about 10² rad/s. Clause 2503. The composition of any one of clauses 1101 to 1219, wherein when formulated in a formulation comprising about 3 ml of fluid and about 1 g of composition, the formulation has a loss modulus between about 10^{3.7} Pa and about 10^{3.8} Pa at an angular frequency between about 10⁻¹ rad/s and about 10² rad/s. Clause 2504. The composition of any one of clauses 1101 to 1219, wherein when formulated in a formulation comprising about 3 ml of fluid and about 1 g of composition, the formulation has a loss modulus between about 10^{3.8} Pa and about 10^{3.9} Pa at an angular frequency between about 10⁻¹ rad/s and about 10² rad/s. Clause 2505. The composition of any one of clauses 1101 to 1219, wherein when formulated in a formulation comprising about 3 ml of fluid and about 1 g of composition, the formulation has a loss modulus between about 10^{3.9} Pa and about 10⁴ Pa at an angular frequency between about 10⁻¹ rad/s and about 10² rad/s. Clause 2506. The composition of any one of clauses 1101 to 1219, wherein when formulated in a formulation comprising about 3 ml of fluid and about 1 g of composition, the formulation has a loss modulus between about 10⁴ Pa and about 10^{4.1} Pa at an angular frequency between about 10⁻¹ rad/s and about 10² rad/s. Clause 2507. The composition of any one of clauses 1101 to 1219, wherein when formulated in a formulation comprising about 3 ml of fluid and about 1 g of composition, the formulation has a loss modulus between about 10^{4.1} Pa and about 10^{4.2} Pa at an angular frequency between about 10⁻¹ rad/s and about 10² rad/s. Clause 2508. The composition of any one of clauses 1101 to 1219, wherein when formulated in a formulation comprising about 3 ml of fluid and about 1 g of composition, the formulation has a loss modulus between about 10^{4.2} Pa and about 10^{4.3} Pa at an angular frequency between about 10⁻¹ rad/s and about 10² rad/s. Clause 2509. The composition of any one of clauses 1101 to 1219, wherein when formulated in a formulation comprising about 3 ml of fluid and about 1 g of composition, the formulation has a loss modulus between about 10^{4.3} Pa and about 10^{4.4} Pa at an angular frequency between about 10⁻¹ rad/s and about 10² rad/s. Clause 2510. The composition of any one of clauses 1101 to 1219, wherein when formulated in a formulation comprising about 3 ml of fluid and about 1 g of composition, the formulation has a loss modulus between about 10^{4.4} Pa and about 10^{4.5} Pa at an angular frequency between about 10⁻¹ rad/s and about 10² rad/s. Clause 2511. The composition of any one of clauses 1101 to 1219, wherein when formulated in a formulation comprising about 3 ml of fluid and about 1 g of composition, the formulation has a loss modulus between about 10^{4.5} Pa and about 10^{4.6} Pa at an angular frequency between about 10⁻¹ rad/s and about 10² rad/s. Clause 2512. The composition of any one of clauses 1101 to 1219, wherein when formulated in a formulation comprising about 3 ml of fluid and about 1 g of composition, the formulation has a loss modulus between about 10^{4.6} Pa and about 10^{4.7} Pa at an angular frequency between about 10⁻¹ rad/s and about 10² rad/s. Clause 2513. The composition of any one of clauses 1101 to 1219, wherein when formulated in a formulation comprising about 3 ml of fluid and about 1 g of composition, the formulation has a loss modulus between about 10^{4.7} Pa and about 10^{4.8} Pa at an angular frequency between about 10⁻¹ rad/s and about 10² rad/s. Clause 2514. The composition of any one of clauses 1101 to 1219, wherein when formulated in a formulation comprising about 3 ml of fluid and about 1 g of composition, the formulation has a loss modulus between about 10^{4.8} Pa and about 10^{4.9} Pa at an angular frequency between about 10⁻¹ rad/s and about 10² rad/s. Clause 2515. The composition of any one of clauses 1101 to 1219, wherein when formulated in a formulation comprising about 3 ml of fluid and about 1 g of composition, the formulation has a loss modulus between about 10^{4.9} Pa and about 10⁵ Pa at an angular frequency between about 10⁻¹ rad/s and about 10² rad/s.

Clause 2600. The composition of any one of clauses 1101 to 1219, wherein when formulated in a formulation comprising about 5 ml of fluid and about 1 g of composition, the formulation has a storage modulus between about 10³ Pa and about 10^{5.5} Pa at an angular frequency between about 10⁻¹ rad/s and about 10² rad/s. Clause 2601. The composition of any one of clauses 1101 to 1219, wherein when formulated in a formulation comprising about 5 ml of fluid and about 1 g of composition, the formulation has a storage modulus between about 10³ Pa and about 10^{3.1} Pa at an angular frequency between about 10⁻¹ rad/s and about 10² rad/s. Clause 2602. The composition of any one of clauses 1101 to 1219, wherein when formulated in a formulation comprising about 5 ml of fluid and about 1 g of composition, the formulation has a storage modulus between about 10^{3.1} Pa and about 10^{3.2} Pa at an angular frequency between about 10⁻¹ rad/s and about 10² rad/s. Clause 2603. The composition of any one of clauses 1101 to 1219, wherein when formulated in a formulation comprising about 5 ml of fluid and about 1 g of composition, the formulation has a storage modulus between about 10^{3.2} Pa and about 10^{3.3} Pa at an angular frequency between about 10⁻¹ rad/s and about 10² rad/s. Clause 2604. The composition of any one of clauses 1101 to 1219, wherein when formulated in a formulation comprising about 5 ml of fluid and about 1 g of composition, the formulation has a storage modulus between about 10^{3.3} Pa and about 10^{3.4} Pa at an angular frequency between about 10⁻¹ rad/s and about 10² rad/s. Clause 2605. The composition of any one of clauses 1101 to 1219, wherein when formulated in a formulation comprising about 5 ml of fluid and about 1 g of composition, the formulation has a storage modulus between about 10^{3.4} Pa and about 10^{3.5} Pa at an angular frequency between about 10⁻¹ rad/s and about 10² rad/s. Clause 2606. The composition of any one of clauses 1101 to 1219, wherein when formulated in a formulation comprising about 5 ml of fluid and about 1 g of composition, the formulation has a storage modulus between about 10^{3.5} Pa and about 10^{3.6} Pa at an angular frequency between about 10⁻¹ rad/s and about 10² rad/s. Clause 2607. The composition of any one of clauses 1101 to 1219, wherein when formulated in a formulation comprising about 5 ml of fluid and about 1 g of composition, the formulation has a storage modulus between about 10^{3.6} Pa and about 10^{3.7} Pa at an angular frequency between about 10⁻¹ rad/s and about 10² rad/s. Clause 2608. The composition of any one of clauses 1101 to 1219, wherein when formulated in a formulation comprising about 5 ml of fluid and about 1 g of composition, the formulation has a storage modulus between about 10^{3.7} Pa and about 10^{3.8} Pa at an angular frequency between about 10⁻¹ rad/s and about 10² rad/s. Clause 2609. The composition of any one of clauses 1101 to 1219, wherein when formulated in a formulation comprising about 5 ml of fluid and about 1 g of composition, the formulation has a storage modulus between about 10^{3.8} Pa and about 10^{3.9} Pa at an angular frequency between about 10⁻¹ rad/s and about 10² rad/s. Clause 2610. The composition of any one of clauses 1101 to 1219, wherein when formulated in a formulation comprising about 5 ml of fluid and about 1 g of composition, the formulation has a storage modulus between about 10^{3.9} Pa and about 10⁴ Pa at an angular frequency between about 10⁻¹ rad/s and about 10² rad/s. Clause 2611. The composition of any one of clauses 1101 to 1219, wherein when formulated in a formulation comprising about 5 ml of fluid and about 1 g of composition, the formulation has a storage modulus between about 10⁴ Pa and about 10^{4.1} Pa at an angular frequency between about 10⁻¹ rad/s and about 10² rad/s. Clause 2612. The composition of any one of clauses 1101 to 1219, wherein when formulated in a formulation comprising about 5 ml of fluid and about 1 g of composition, the formulation has a storage modulus between about 10^{4.1} Pa and about 10^{4.2} Pa at an angular frequency between about 10⁻¹ rad/s and about 10² rad/s. Clause 2613. The composition of any one of clauses 1101 to 1219, wherein when formulated in a formulation comprising about 5 ml of fluid and about 1 g of composition, the formulation has a storage modulus between about 10^{4.2} Pa and about 10^{4.3} Pa at an angular frequency between about 10⁻¹ rad/s and about 10² rad/s. Clause 2614. The composition of any one of clauses 1101 to 1219, wherein when formulated in a formulation comprising about 5 ml of fluid and about 1 g of composition, the formulation has a storage modulus between about 10^{4.3} Pa and about 10^{4.4} Pa at an angular frequency between about 10⁻¹ rad/s and about 10² rad/s. Clause 2615. The composition of any one of clauses 1101 to 1219, wherein when formulated in a formulation comprising about 5 ml of fluid and about 1 g of composition, the formulation has a storage modulus between about 10^{4.4} Pa and about 10^{4.5} Pa at an angular frequency between about 10⁻¹ rad/s and about 10² rad/s. Clause 2616. The composition of any one of clauses 1101 to 1219, wherein when formulated in a formulation comprising about 5 ml of fluid and about 1 g of composition, the formulation has a storage modulus between about 10^{4.5} Pa and about 10^{4.6} Pa at an angular frequency between about 10⁻¹ rad/s and about 10² rad/s. Clause 2617. The composition of any one of clauses 1101 to 1219, wherein when formulated in a formulation comprising about 5 ml of fluid and about 1 g of composition, the formulation has a storage modulus between about 10^{4.6} Pa and about 10^{4.7} Pa at an angular frequency between about 10⁻¹ rad/s and about 10² rad/s. Clause 2618. The composition of any one of clauses 1101 to 1219, wherein when formulated in a formulation comprising about 5 ml of fluid and about 1 g of composition, the formulation has a storage modulus between about 10^{4.7} Pa and about 10^{4.8} Pa at an angular frequency between about 10⁻¹ rad/s and about 10² rad/s. Clause 2619. The composition of any one of clauses 1101 to 1219, wherein when formulated in a formulation comprising about 5 ml of fluid and about 1 g of composition, the formulation has a storage modulus between about 10^{4.8} Pa and about 10^{4.9} Pa at an angular frequency between about 10⁻¹ rad/s and about 10² rad/s. Clause 2620. The composition of any one of clauses 1101 to 1219, wherein when formulated in a formulation comprising about 5 ml of fluid and about 1 g of composition, the formulation has a storage modulus between about 10^{4.9} Pa and about 10⁵ Pa at an angular frequency between about 10⁻¹ rad/s and about 10² rad/s. Clause 2621. The composition of any one of clauses 1101 to 1219, wherein when formulated in a formulation comprising about 5 ml of fluid and about 1 g of composition, the formulation has a storage modulus between about 10⁵ Pa and about 10^{5.1} Pa at an angular frequency between about 10⁻¹ rad/s and about 10² rad/s. Clause 2622. The composition of any one of clauses 1101 to 1219, wherein when formulated in a formulation comprising about 5 ml of fluid and about 1 g of composition, the formulation has a storage modulus between about 10^{5.1} Pa and about 10^{5.2} Pa at an angular frequency between about 10⁻¹ rad/s and about 10² rad/s. Clause 2623. The composition of any one of clauses 1101 to 1219, wherein when formulated in a formulation comprising about 5 ml of fluid and about 1 g of composition, the formulation has a storage modulus between about 10^{5.2} Pa and about 10^{5.3} Pa at an angular frequency between about 10⁻¹ rad/s and about 10² rad/s. Clause 2624. The composition of any one of clauses 1101 to 1219, wherein when formulated in a formulation comprising about 5 ml of fluid and about 1 g of composition, the formulation has a storage modulus between about 10^{5.3} Pa and about 10^{5.4} Pa at an angular frequency between about 10⁻¹ rad/s and about 10² rad/s. Clause 2625. The composition of any one of clauses 1101 to 1219, wherein when formulated in a formulation comprising about 5 ml of fluid and about 1 g of composition, the formulation has a storage modulus between about 10^{5.4} Pa and about 10^{5.5} Pa at an angular frequency between about 10⁻¹ rad/s and about 10² rad/s.

Clause 2700. The composition of any one of clauses 1101 to 1219, wherein when formulated in a formulation comprising about 5 ml of fluid and about 1 g of composition, the formulation has a loss modulus between about 10³ Pa and about 10⁵ Pa at an angular frequency between about 10⁻¹ rad/s and about 10² rad/s. Clause 2701. The composition of any one of clauses 1101 to 1219, wherein when formulated in a formulation comprising about 5 ml of fluid and about 1 g of composition, the formulation has a loss modulus between about 10³ Pa and about 10^{3.1} Pa at an angular frequency between about 10⁻¹ rad/s and about 10² rad/s. Clause 2702. The composition of any one of clauses 1101 to 1219, wherein when formulated in a formulation comprising about 5 ml of fluid and about 1 g of composition, the formulation has a loss modulus between about 10^{3.1} Pa and about 10^{3.2} Pa at an angular frequency between about 10⁻¹ rad/s and about 10² rad/s. Clause 2703. The composition of any one of clauses 1101 to 1219, wherein when formulated in a formulation comprising about 5 ml of fluid and about 1 g of composition, the formulation has a loss modulus between about 10^{3.2} Pa and about 10^{3.3} Pa at an angular frequency between about 10⁻¹ rad/s and about 10² rad/s. Clause 2704. The composition of any one of clauses 1101 to 1219, wherein when formulated in a formulation comprising about 5 ml of fluid and about 1 g of composition, the formulation has a loss modulus between about 10^{3.3} Pa and about 10^{3.4} Pa at an angular frequency between about 10⁻¹ rad/s and about 10² rad/s. Clause 2705. The composition of any one of clauses 1101 to 1219, wherein when formulated in a formulation comprising about 5 ml of fluid and about 1 g of composition, the formulation has a loss modulus between about 10^{3.4} Pa and about 10^{3.5} Pa at an angular frequency between about 10⁻¹ rad/s and about 10² rad/s. Clause 2706. The composition of any one of clauses 1101 to 1219, wherein when formulated in a formulation comprising about 5 ml of fluid and about 1 g of composition, the formulation has a loss modulus between about 10^{3.5} Pa and about 10^{3.6} Pa at an angular frequency between about 10⁻¹ rad/s and about 10² rad/s. Clause 2707. The composition of any one of clauses 1101 to 1219, wherein when formulated in a formulation comprising about 5 ml of fluid and about 1 g of composition, the formulation has a loss modulus between about 10^{3.6} Pa and about 10^{3.7} Pa at an angular frequency between about 10⁻¹ rad/s and about 10² rad/s. Clause 2708. The composition of any one of clauses 1101 to 1219, wherein when formulated in a formulation comprising about 5 ml of fluid and about 1 g of composition, the formulation has a loss modulus between about 10^{3.7} Pa and about 10^{3.8} Pa at an angular frequency between about 10⁻¹ rad/s and about 10² rad/s. Clause 2709. The composition of any one of clauses 1101 to 1219, wherein when formulated in a formulation comprising about 5 ml of fluid and about 1 g of composition, the formulation has a loss modulus between about 10^{3.8} Pa and about 10^{3.9} Pa at an angular frequency between about 10⁻¹ rad/s and about 10² rad/s. Clause 2710. The composition of any one of clauses 1101 to 1219, wherein when formulated in a formulation comprising about 5 ml of fluid and about 1 g of composition, the formulation has a loss modulus between about 10^{3.9} Pa and about 10⁴ Pa at an angular frequency between about 10⁻¹ rad/s and about 10² rad/s. Clause 2711. The composition of any one of clauses 1101 to 1219, wherein when formulated in a formulation comprising about 5 ml of fluid and about 1 g of composition, the formulation has a loss modulus between about 10⁴ Pa and about 10^{4.1} Pa at an angular frequency between about 10⁻¹ rad/s and about 10² rad/s. Clause 2712. The composition of any one of clauses 1101 to 1219, wherein when formulated in a formulation comprising about 5 ml of fluid and about 1 g of composition, the formulation has a loss modulus between about 10^{4.1} Pa and about 10^{4.2} Pa at an angular frequency between about 10⁻¹ rad/s and about 10² rad/s. Clause 2713. The composition of any one of clauses 1101 to 1219, wherein when formulated in a formulation comprising about 5 ml of fluid and about 1 g of composition, the formulation has a loss modulus between about 10^{4.2} Pa and about 10^{4.3} Pa at an angular frequency between about 10⁻¹ rad/s and about 10² rad/s. Clause 2714. The composition of any one of clauses 1101 to 1219, wherein when formulated in a formulation comprising about 5 ml of fluid and about 1 g of composition, the formulation has a loss modulus between about 10^{4.3} Pa and about 10^{4.4} Pa at an angular frequency between about 10⁻¹ rad/s and about 10² rad/s. Clause 2715. The composition of any one of clauses 1101 to 1219, wherein when formulated in a formulation comprising about 5 ml of fluid and about 1 g of composition, the formulation has a loss modulus between about 10^{4.4} Pa and about 10^{4.5} Pa at an angular frequency between about 10⁻¹ rad/s and about 10² rad/s. Clause 2716. The composition of any one of clauses 1101 to 1219, wherein when formulated in a formulation comprising about 5 ml of fluid and about 1 g of composition, the formulation has a loss modulus between about 10^{4.5} Pa and about 10^{4.6} Pa at an angular frequency between about 10⁻¹ rad/s and about 10² rad/s. Clause 2717. The composition of any one of clauses 1101 to 1219, wherein when formulated in a formulation comprising about 5 ml of fluid and about 1 g of composition, the formulation has a loss modulus between about 10^{4.6} Pa and about 10^{4.7} Pa at an angular frequency between about 10⁻¹ rad/s and about 10² rad/s. Clause 2718. The composition of any one of clauses 1101 to 1219, wherein when formulated in a formulation comprising about 5 ml of fluid and about 1 g of composition, the formulation has a loss modulus between about 10^{4.7} Pa and about 10^{4.8} Pa at an angular frequency between about 10⁻¹ rad/s and about 10² rad/s. Clause 2719. The composition of any one of clauses 1101 to 1219, wherein when formulated in a formulation comprising about 5 ml of fluid and about 1 g of composition, the formulation has a loss modulus between about 10^{4.8} Pa and about 10^{4.9} Pa at an angular frequency between about 10⁻¹ rad/s and about 10² rad/s. Clause 2720. The composition of any one of clauses 1101 to 1219, wherein when formulated in a formulation comprising about 5 ml of fluid and about 1 g of composition, the formulation has a loss modulus between about 10^{4.9} Pa and about 10⁵ Pa at an angular frequency between about 10⁻¹ rad/s and about 10² rad/s.

Clause 2800. The composition of any one of clauses 1101 to 1219, wherein when formulated in a formulation comprising about 7 ml of fluid and about 1 g of composition, the formulation has a storage modulus between about 10^{2.5} Pa and about 10^{4.5} Pa at an angular frequency between about 10⁻¹ rad/s and about 10² rad/s. Clause 2801. The composition of any one of clauses 1101 to 1219, wherein when formulated in a formulation comprising about 7 ml of fluid and about 1 g of composition, the formulation has a storage modulus between about 10^{2.5} Pa and about 10^{2.6} Pa at an angular frequency between about 10⁻¹ rad/s and about 10² rad/s. Clause 2802. The composition of any one of clauses 1101 to 1219, wherein when formulated in a formulation comprising about 7 ml of fluid and about 1 g of composition, the formulation has a storage modulus between about 10^{2.6} Pa and about 10^{2.7} Pa at an angular frequency between about 10⁻¹ rad/s and about 10² rad/s. Clause 2803. The composition of any one of clauses 1101 to 1219, wherein when formulated in a formulation comprising about 7 ml of fluid and about 1 g of composition, the formulation has a storage modulus between about 10^{2.7} Pa and about 10^{2.8} Pa at an angular frequency between about 10⁻¹ rad/s and about 10² rad/s. Clause 2804. The composition of any one of clauses 1101 to 1219, wherein when formulated in a formulation comprising about 7 ml of fluid and about 1 g of composition, the formulation has a storage modulus between about 10^{2.8} Pa and about 10^{2.9} Pa at an angular frequency between about 10⁻¹ rad/s and about 10² rad/s. Clause 2805. The composition of any one of clauses 1101 to 1219, wherein when formulated in a formulation comprising about 7 ml of fluid and about 1 g of composition, the formulation has a storage modulus between about 10^{2.9} Pa and about 10³ Pa at an angular frequency between about 10⁻¹ rad/s and about 10² rad/s. Clause 2806. The composition of any one of clauses 1101 to 1219, wherein when formulated in a formulation comprising about 7 ml of fluid and about 1 g of composition, the formulation has a storage modulus between about 10³ Pa and about 10^{3.1} Pa at an angular frequency between about 10⁻¹ rad/s and about 10² rad/s. Clause 2807. The composition of any one of clauses 1101 to 1219, wherein when formulated in a formulation comprising about 7 ml of fluid and about 1 g of composition, the formulation has a storage modulus between about 10^{3.1} Pa and about 10^{3.2} Pa at an angular frequency between about 10⁻¹ rad/s and about 10² rad/s. Clause 2808. The composition of any one of clauses 1101 to 1219, wherein when formulated in a formulation comprising about 7 ml of fluid and about 1 g of composition, the formulation has a storage modulus between about 10^{3.2} Pa and about 10^{3.3} Pa at an angular frequency between about 10⁻¹ rad/s and about 10² rad/s. Clause 2809. The composition of any one of clauses 1101 to 1219, wherein when formulated in a formulation comprising about 7 ml of fluid and about 1 g of composition, the formulation has a storage modulus between about 10^{3.3} Pa and about 10^{3.4} Pa at an angular frequency between about 10⁻¹ rad/s and about 10² rad/s. Clause 2810. The composition of any one of clauses 1101 to 1219, wherein when formulated in a formulation comprising about 7 ml of fluid and about 1 g of composition, the formulation has a storage modulus between about 10^{3.4} Pa and about 10^{3.5} Pa at an angular frequency between about 10⁻¹ rad/s and about 10² rad/s. Clause 2811. The composition of any one of clauses 1101 to 1219, wherein when formulated in a formulation comprising about 7 ml of fluid and about 1 g of composition, the formulation has a storage modulus between about 10^{3.5} Pa and about 10^{3.6} Pa at an angular frequency between about 10⁻¹ rad/s and about 10² rad/s. Clause 2812. The composition of any one of clauses 1101 to 1219, wherein when formulated in a formulation comprising about 7 ml of fluid and about 1 g of composition, the formulation has a storage modulus between about 10^{3.6} Pa and about 10^{3.7} Pa at an angular frequency between about 10⁻¹ rad/s and about 10² rad/s. Clause 2813. The composition of any one of clauses 1101 to 1219, wherein when formulated in a formulation comprising about 7 ml of fluid and about 1 g of composition, the formulation has a storage modulus between about 10^{3.7} Pa and about 10^{3.8} Pa at an angular frequency between about 10⁻¹ rad/s and about 10² rad/s. Clause 2814. The composition of any one of clauses 1101 to 1219, wherein when formulated in a formulation comprising about 7 ml of fluid and about 1 g of composition, the formulation has a storage modulus between about 10^{3.8} Pa and about 10^{3.9} Pa at an angular frequency between about 10⁻¹ rad/s and about 10² rad/s. Clause 2815. The composition of any one of clauses 1101 to 1219, wherein when formulated in a formulation comprising about 7 ml of fluid and about 1 g of composition, the formulation has a storage modulus between about 10^{3.9} Pa and about 10⁴ Pa at an angular frequency between about 10⁻¹ rad/s and about 10² rad/s. Clause 2816. The composition of any one of clauses 1101 to 1219, wherein when formulated in a formulation comprising about 7 ml of fluid and about 1 g of composition, the formulation has a storage modulus between about 10⁴ Pa and about 10^{4.1} Pa at an angular frequency between about 10⁻¹ rad/s and about 10² rad/s. Clause 2817. The composition of any one of clauses 1101 to 1219, wherein when formulated in a formulation comprising about 7 ml of fluid and about 1 g of composition, the formulation has a storage modulus between about 10^{4.1} Pa and about 10^{4.2} Pa at an angular frequency between about 10⁻¹ rad/s and about 10² rad/s. Clause 2818. The composition of any one of clauses 1101 to 1219, wherein when formulated in a formulation comprising about 7 ml of fluid and about 1 g of composition, the formulation has a storage modulus between about 10^{4.2} Pa and about 10^{4.3} Pa at an angular frequency between about 10⁻¹ rad/s and about 10² rad/s. Clause 2819. The composition of any one of clauses 1101 to 1219, wherein when formulated in a formulation comprising about 7 ml of fluid and about 1 g of composition, the formulation has a storage modulus between about 10^{4.3} Pa and about 10^{4.4} Pa at an angular frequency between about 10⁻¹ rad/s and about 10² rad/s. Clause 2820. The composition of any one of clauses 1101 to 1219, wherein when formulated in a formulation comprising about 7 ml of fluid and about 1 g of composition, the formulation has a storage modulus between about 10^{4.4} Pa and about 10^{4.5} Pa at an angular frequency between about 10⁻¹ rad/s and about 10² rad/s.

Clause 2900. The composition of any one of clauses 1101 to 1219, wherein when formulated in a formulation comprising about 7 ml of fluid and about 1 g of composition, the formulation has a loss modulus between about 10² Pa and about 10⁴ Pa at an angular frequency between about 10⁻¹ rad/s and about 10² rad/s. Clause 2901. The composition of any one of clauses 1101 to 1219, wherein when formulated in a formulation comprising about 7 ml of fluid and about 1 g of composition, the formulation has a loss modulus between about 10² Pa and about 10^{2.1} Pa at an angular frequency between about 10⁻¹ rad/s and about 10² rad/s. Clause 2902. The composition of any one of clauses 1101 to 1219, wherein when formulated in a formulation comprising about 7 ml of fluid and about 1 g of composition, the formulation has a loss modulus between about 10^{2.1} Pa and about 10^{2.2} Pa at an angular frequency between about 10⁻¹ rad/s and about 10² rad/s. Clause 2903. The composition of any one of clauses 1101 to 1219, wherein when formulated in a formulation comprising about 7 ml of fluid and about 1 g of composition, the formulation has a loss modulus between about 10^{2.2} Pa and about 10^{2.3} Pa at an angular frequency between about 10⁻¹ rad/s and about 10² rad/s. Clause 2904. The composition of any one of clauses 1101 to 1219, wherein when formulated in a formulation comprising about 7 ml of fluid and about 1 g of composition, the formulation has a loss modulus between about 10^{2.3} Pa and about 10^{2.4} Pa at an angular frequency between about 10⁻¹ rad/s and about 10² rad/s. Clause 2905. The composition of any one of clauses 1101 to 1219, wherein when formulated in a formulation comprising about 7 ml of fluid and about 1 g of composition, the formulation has a loss modulus between about 10^{2.4} Pa and about 10^{2.5} Pa at an angular frequency between about 10⁻¹ rad/s and about 10² rad/s. Clause 2906. The composition of any one of clauses 1101 to 1219, wherein when formulated in a formulation comprising about 7 ml of fluid and about 1 g of composition, the formulation has a loss modulus between about 10^{2.5} Pa and about 10^{2.6} Pa at an angular frequency between about 10⁻¹ rad/s and about 10² rad/s. Clause 2907. The composition of any one of clauses 1101 to 1219, wherein when formulated in a formulation comprising about 7 ml of fluid and about 1 g of composition, the formulation has a loss modulus between about 10^{2.6} Pa and about 10^{2.7} Pa at an angular frequency between about 10⁻¹ rad/s and about 10² rad/s. Clause 2908. The composition of any one of clauses 1101 to 1219, wherein when formulated in a formulation comprising about 7 ml of fluid and about 1 g of composition, the formulation has a loss modulus between about 10^{2.7} Pa and about 10^{2.8} Pa at an angular frequency between about 10⁻¹ rad/s and about 10² rad/s. Clause 2909. The composition of any one of clauses 1101 to 1219, wherein when formulated in a formulation comprising about 7 ml of fluid and about 1 g of composition, the formulation has a loss modulus between about 10^{2.8} Pa and about 10^{2.9} Pa at an angular frequency between about 10⁻¹ rad/s and about 10² rad/s. Clause 2910. The composition of any one of clauses 1101 to 1219, wherein when formulated in a formulation comprising about 7 ml of fluid and about 1 g of composition, the formulation has a loss modulus between about 10^{2.9} Pa and about 10³ Pa at an angular frequency between about 10⁻¹ rad/s and about 10² rad/s. Clause 2911. The composition of any one of clauses 1101 to 1219, wherein when formulated in a formulation comprising about 7 ml of fluid and about 1 g of composition, the formulation has a loss modulus between about 10³ Pa and about 10^{3.1} Pa at an angular frequency between about 10⁻¹ rad/s and about 10² rad/s. Clause 2912. The composition of any one of clauses 1101 to 1219, wherein when formulated in a formulation comprising about 7 ml of fluid and about 1 g of composition, the formulation has a loss modulus between about 10^{3.1} Pa and about 10^{3.2} Pa at an angular frequency between about 10⁻¹ rad/s and about 10² rad/s. Clause 2913. The composition of any one of clauses 1101 to 1219, wherein when formulated in a formulation comprising about 7 ml of fluid and about 1 g of composition, the formulation has a loss modulus between about 10^{3.2} Pa and about 10^{3.3} Pa at an angular frequency between about 10⁻¹ rad/s and about 10² rad/s. Clause 2914. The composition of any one of clauses 1101 to 1219, wherein when formulated in a formulation comprising about 7 ml of fluid and about 1 g of composition, the formulation has a loss modulus between about 10^{3.3} Pa and about 10^{3.4} Pa at an angular frequency between about 10⁻¹ rad/s and about 10² rad/s. Clause 2915. The composition of any one of clauses 1101 to 1219, wherein when formulated in a formulation comprising about 7 ml of fluid and about 1 g of composition, the formulation has a loss modulus between about 10^{3.4} Pa and about 10^{3.5} Pa at an angular frequency between about 10⁻¹ rad/s and about 10² rad/s. Clause 2916. The composition of any one of clauses 1101 to 1219, wherein when formulated in a formulation comprising about 7 ml of fluid and about 1 g of composition, the formulation has a loss modulus between about 10^{3.5} Pa and about 10^{3.6} Pa at an angular frequency between about 10⁻¹ rad/s and about 10² rad/s. Clause 2917. The composition of any one of clauses 1101 to 1219, wherein when formulated in a formulation comprising about 7 ml of fluid and about 1 g of composition, the formulation has a loss modulus between about 10^{3.6} Pa and about 10^{3.7} Pa at an angular frequency between about 10⁻¹ rad/s and about 10² rad/s. Clause 2918. The composition of any one of clauses 1101 to 1219, wherein when formulated in a formulation comprising about 7 ml of fluid and about 1 g of composition, the formulation has a loss modulus between about 10^{3.7} Pa and about 10^{3.8} Pa at an angular frequency between about 10⁻¹ rad/s and about 10² rad/s. Clause 2919. The composition of any one of clauses 1101 to 1219, wherein when formulated in a formulation comprising about 7 ml of fluid and about 1 g of composition, the formulation has a loss modulus between about 10^{3.8} Pa and about 10^{3.9} Pa at an angular frequency between about 10⁻¹ rad/s and about 10² rad/s. Clause 2920. The composition of any one of clauses 1101 to 1219, wherein when formulated in a formulation comprising about 7 ml of fluid and about 1 g of composition, the formulation has a loss modulus between about 10^{3.9} Pa and about 10⁴ Pa at an angular frequency between about 10⁻¹ rad/s and about 10² rad/s.

Clause 3000. The composition of any one of clauses 1101 to 1219, wherein when formulated in a formulation comprising about 3 ml of fluid and about 1 g of composition, the formulation has a storage modulus between about 10^{3.5} Pa and about 10^{5.5} Pa at an angular frequency between about 10⁻¹ rad/s and about 10² rad/s. Clause 3001. The composition of any one of clauses 1101 to 1219, wherein when formulated in a formulation comprising about 3 ml of fluid and about 1 g of composition, the formulation has a storage modulus between about 10^{3.5} Pa and about 10^{3.6} Pa at an angular frequency between about 10⁻¹ rad/s and about 10² rad/s. Clause 3002. The composition of any one of clauses 1101 to 1219, wherein when formulated in a formulation comprising about 3 ml of fluid and about 1 g of composition, the formulation has a storage modulus between about 10^{3.6} Pa and about 10^{3.7} Pa at an angular frequency between about 10⁻¹ rad/s and about 10² rad/s. Clause 3003. The composition of any one of clauses 1101 to 1219, wherein when formulated in a formulation comprising about 3 ml of fluid and about 1 g of composition, the formulation has a storage modulus between about 10^{3.7} Pa and about 10^{3.8} Pa at an angular frequency between about 10⁻¹ rad/s and about 10² rad/s. Clause 3004. The composition of any one of clauses 1101 to 1219, wherein when formulated in a formulation comprising about 3 ml of fluid and about 1 g of composition, the formulation has a storage modulus between about 10^{3.8} Pa and about 10^{3.9} Pa at an angular frequency between about 10⁻¹ rad/s and about 10² rad/s. Clause 3005. The composition of any one of clauses 1101 to 1219, wherein when formulated in a formulation comprising about 3 ml of fluid and about 1 g of composition, the formulation has a storage modulus between about 10^{3.9} Pa and about 10⁴ Pa at an angular frequency between about 10⁻¹ rad/s and about 10² rad/s. Clause 3006. The composition of any one of clauses 1101 to 1219, wherein when formulated in a formulation comprising about 3 ml of fluid and about 1 g of composition, the formulation has a storage modulus between about 10⁴ Pa and about 10^{4.1} Pa at an angular frequency between about 10⁻¹ rad/s and about 10² rad/s. Clause 3007. The composition of any one of clauses 1101 to 1219, wherein when formulated in a formulation comprising about 3 ml of fluid and about 1 g of composition, the formulation has a storage modulus between about 10^{4.1} Pa and about 10^{4.2} Pa at an angular frequency between about 10⁻¹ rad/s and about 10² rad/s. Clause 3008. The composition of any one of clauses 1101 to 1219, wherein when formulated in a formulation comprising about 3 ml of fluid and about 1 g of composition, the formulation has a storage modulus between about 10^{4.2} Pa and about 10^{4.3} Pa at an angular frequency between about 10⁻¹ rad/s and about 10² rad/s. Clause 3009. The composition of any one of clauses 1101 to 1219, wherein when formulated in a formulation comprising about 3 ml of fluid and about 1 g of composition, the formulation has a storage modulus between about 10^{4.3} Pa and about 10^{4.4} Pa at an angular frequency between about 10⁻¹ rad/s and about 10² rad/s. Clause 3010. The composition of any one of clauses 1101 to 1219, wherein when formulated in a formulation comprising about 3 ml of fluid and about 1 g of composition, the formulation has a storage modulus between about 10^{4.4} Pa and about 10^{4.5} Pa at an angular frequency between about 10⁻¹ rad/s and about 10² rad/s. Clause 3011. The composition of any one of clauses 1101 to 1219, wherein when formulated in a formulation comprising about 3 ml of fluid and about 1 g of composition, the formulation has a storage modulus between about 10^{4.5} Pa and about 10^{4.6} Pa at an angular frequency between about 10⁻¹ rad/s and about 10² rad/s. Clause 3012. The composition of any one of clauses 1101 to 1219, wherein when formulated in a formulation comprising about 3 ml of fluid and about 1 g of composition, the formulation has a storage modulus between about 10^{4.6} Pa and about 10^{4.7} Pa at an angular frequency between about 10⁻¹ rad/s and about 10² rad/s. Clause 3013. The composition of any one of clauses 1101 to 1219, wherein when formulated in a formulation comprising about 3 ml of fluid and about 1 g of composition, the formulation has a storage modulus between about 10^{4.7} Pa and about 10^{4.8} Pa at an angular frequency between about 10⁻¹ rad/s and about 10² rad/s. Clause 3014. The composition of any one of clauses 1101 to 1219, wherein when formulated in a formulation comprising about 3 ml of fluid and about 1 g of composition, the formulation has a storage modulus between about 10^{4.8} Pa and about 10^{4.9} Pa at an angular frequency between about 10⁻¹ rad/s and about 10² rad/s. Clause 3015. The composition of any one of clauses 1101 to 1219, wherein when formulated in a formulation comprising about 3 ml of fluid and about 1 g of composition, the formulation has a storage modulus between about 10^{4.9} Pa and about 10⁵ Pa at an angular frequency between about 10⁻¹ rad/s and about 10² rad/s. Clause 3016. The composition of any one of clauses 1101 to 1219, wherein when formulated in a formulation comprising about 3 ml of fluid and about 1 g of composition, the formulation has a storage modulus between about 10⁵ Pa and about 10^{5.1} Pa at an angular frequency between about 10⁻¹ rad/s and about 10² rad/s. Clause 3017. The composition of any one of clauses 1101 to 1219, wherein when formulated in a formulation comprising about 3 ml of fluid and about 1 g of composition, the formulation has a storage modulus between about 10^{5.1} Pa and about 10^{5.2} Pa at an angular frequency between about 10⁻¹ rad/s and about 10² rad/s. Clause 3018. The composition of any one of clauses 1101 to 1219, wherein when formulated in a formulation comprising about 3 ml of fluid and about 1 g of composition, the formulation has a storage modulus between about 10^{5.2} Pa and about 10^{5.3} Pa at an angular frequency between about 10⁻¹ rad/s and about 10² rad/s. Clause 3019. The composition of any one of clauses 1101 to 1219, wherein when formulated in a formulation comprising about 3 ml of fluid and about 1 g of composition, the formulation has a storage modulus between about 10^{5.3} Pa and about 10^{5.4} Pa at an angular frequency between about 10⁻¹ rad/s and about 10² rad/s. Clause 3020. The composition of any one of clauses 1101 to 1219, wherein when formulated in a formulation comprising about 3 ml of fluid and about 1 g of composition, the formulation has a storage modulus between about 10^{5.4} Pa and about 10⁵ Pa at an angular frequency between about 10⁻¹ rad/s and about 10² rad/s.

Clause 3100. The composition of any one of clauses 1101 to 1219, wherein when formulated in a formulation comprising about 3 ml of fluid and about 1 g of composition, the formulation has a loss modulus between about 10³ Pa and about 10⁵ Pa at an angular frequency between about 10⁻¹ rad/s and about 10² rad/s. Clause 3101. The composition of any one of clauses 1101 to 1219, wherein when formulated in a formulation comprising about 3 ml of fluid and about 1 g of composition, the formulation has a loss modulus between about 10³ Pa and about 10^{3.1} Pa at an angular frequency between about 10⁻¹ rad/s and about 10² rad/s. Clause 3102. The composition of any one of clauses 1101 to 1219, wherein when formulated in a formulation comprising about 3 ml of fluid and about 1 g of composition, the formulation has a loss modulus between about 10^{3.1} Pa and about 10^{3.2} Pa at an angular frequency between about 10⁻¹ rad/s and about 10² rad/s. Clause 3103. The composition of any one of clauses 1101 to 1219, wherein when formulated in a formulation comprising about 3 ml of fluid and about 1 g of composition, the formulation has a loss modulus between about 10^{3.2} Pa and about 10^{3.3} Pa at an angular frequency between about 10⁻¹ rad/s and about 10² rad/s. Clause 3104. The composition of any one of clauses 1101 to 1219, wherein when formulated in a formulation comprising about 3 ml of fluid and about 1 g of composition, the formulation has a loss modulus between about 10^{3.3} Pa and about 10^{3.4} Pa at an angular frequency between about 10⁻¹ rad/s and about 10² rad/s. Clause 3105. The composition of any one of clauses 1101 to 1219, wherein when formulated in a formulation comprising about 3 ml of fluid and about 1 g of composition, the formulation has a loss modulus between about 10^{3.4} Pa and about 10^{3.5} Pa at an angular frequency between about 10⁻¹ rad/s and about 10² rad/s. Clause 3106. The composition of any one of clauses 1101 to 1219, wherein when formulated in a formulation comprising about 3 ml of fluid and about 1 g of composition, the formulation has a loss modulus between about 10^{3.5} Pa and about 10^{3.6} Pa at an angular frequency between about 10⁻¹ rad/s and about 10² rad/s. Clause 3107. The composition of any one of clauses 1101 to 1219, wherein when formulated in a formulation comprising about 3 ml of fluid and about 1 g of composition, the formulation has a loss modulus between about 10^{3.6} Pa and about 10^{3.7} Pa at an angular frequency between about 10⁻¹ rad/s and about 10² rad/s. Clause 3108. The composition of any one of clauses 1101 to 1219, wherein when formulated in a formulation comprising about 3 ml of fluid and about 1 g of composition, the formulation has a loss modulus between about 10^{3.7} Pa and about 10^{3.8} Pa at an angular frequency between about 10⁻¹ rad/s and about 10² rad/s. Clause 3109. The composition of any one of clauses 1101 to 1219, wherein when formulated in a formulation comprising about 3 ml of fluid and about 1 g of composition, the formulation has a loss modulus between about 10^{3.8} Pa and about 10^{3.9} Pa at an angular frequency between about 10⁻¹ rad/s and about 10² rad/s. Clause 3110. The composition of any one of clauses 1101 to 1219, wherein when formulated in a formulation comprising about 3 ml of fluid and about 1 g of composition, the formulation has a loss modulus between about 10^{3.9} Pa and about 10⁴ Pa at an angular frequency between about 10⁻¹ rad/s and about 10² rad/s. Clause 3111. The composition of any one of clauses 1101 to 1219, wherein when formulated in a formulation comprising about 3 ml of fluid and about 1 g of composition, the formulation has a loss modulus between about 10⁴ Pa and about 10^{4.1} Pa at an angular frequency between about 10⁻¹ rad/s and about 10² rad/s. Clause 3112. The composition of any one of clauses 1101 to 1219, wherein when formulated in a formulation comprising about 3 ml of fluid and about 1 g of composition, the formulation has a loss modulus between about 10^{4.1} Pa and about 10^{4.2} Pa at an angular frequency between about 10⁻¹ rad/s and about 10² rad/s. Clause 3113. The composition of any one of clauses 1101 to 1219, wherein when formulated in a formulation comprising about 3 ml of fluid and about 1 g of composition, the formulation has a loss modulus between about 10^{4.2} Pa and about 10^{4.3} Pa at an angular frequency between about 10⁻¹ rad/s and about 10² rad/s. Clause 3114. The composition of any one of clauses 1101 to 1219, wherein when formulated in a formulation comprising about 3 ml of fluid and about 1 g of composition, the formulation has a loss modulus between about 10^{4.3} Pa and about 10^{4.4} Pa at an angular frequency between about 10⁻¹ rad/s and about 10² rad/s. Clause 3115. The composition of any one of clauses 1101 to 1219, wherein when formulated in a formulation comprising about 3 ml of fluid and about 1 g of composition, the formulation has a loss modulus between about 10^{4.4} Pa and about 10^{4.5} Pa at an angular frequency between about 10⁻¹ rad/s and about 10² rad/s. Clause 3116. The composition of any one of clauses 1101 to 1219, wherein when formulated in a formulation comprising about 3 ml of fluid and about 1 g of composition, the formulation has a loss modulus between about 10^{4.5} Pa and about 10^{4.6} Pa at an angular frequency between about 10⁻¹ rad/s and about 10² rad/s. Clause 3117. The composition of any one of clauses 1101 to 1219, wherein when formulated in a formulation comprising about 3 ml of fluid and about 1 g of composition, the formulation has a loss modulus between about 10^{4.6} Pa and about 10^{4.7} Pa at an angular frequency between about 10⁻¹ rad/s and about 10² rad/s. Clause 3118. The composition of any one of clauses 1101 to 1219, wherein when formulated in a formulation comprising about 3 ml of fluid and about 1 g of composition, the formulation has a loss modulus between about 10^{4.7} Pa and about 10^{4.8} Pa at an angular frequency between about 10⁻¹ rad/s and about 10² rad/s. Clause 3119. The composition of any one of clauses 1101 to 1219, wherein when formulated in a formulation comprising about 3 ml of fluid and about 1 g of composition, the formulation has a loss modulus between about 10^{4.8} Pa and about 10^{4.9} Pa at an angular frequency between about 10⁻¹ rad/s and about 10² rad/s. Clause 3120. The composition of any one of clauses 1101 to 1219, wherein when formulated in a formulation comprising about 3 ml of fluid and about 1 g of composition, the formulation has a loss modulus between about 10^{4.9} Pa and about 10⁵ Pa at an angular frequency between about 10⁻¹ rad/s and about 10² rad/s.

Clause 3200. The composition of any one of clauses 1101 to 3120, wherein the formulation has a phase angle plateau between about 5° and about 25°. Clause 3201. The composition of any one of clauses 1101 to 3120, wherein the formulation has a phase angle plateau of about 3°. Clause 3202. The composition of any one of clauses 1101 to 3120, wherein the formulation has a phase angle plateau of about 4°. Clause 3203. The composition of any one of clauses 1101 to 3120, wherein the formulation has a phase angle plateau of about 5°. Clause 3204. The composition of any one of clauses 1101 to 3120, wherein the formulation has a phase angle plateau of about 6°. Clause 3205. The composition of any one of clauses 1101 to 3120, wherein the formulation has a phase angle plateau of about 7°. Clause 3206. The composition of any one of clauses 1101 to 3120, wherein the formulation has a phase angle plateau of about 8°. Clause 3207. The composition of any one of clauses 1101 to 3120, wherein the formulation has a phase angle plateau of about 9°. Clause 3208. The composition of any one of clauses 1101 to 3120, wherein the formulation has a phase angle plateau of about 10°. Clause 3209. The composition of any one of clauses 1101 to 3120, wherein the formulation has a phase angle plateau of about 11°. Clause 3210. The composition of any one of clauses 1101 to 3120, wherein the formulation has a phase angle plateau of about 12°. Clause 3211. The composition of any one of clauses 1101 to 3120, wherein the formulation has a phase angle plateau of about 13°. Clause 3212. The composition of any one of clauses 1101 to 3120, wherein the formulation has a phase angle plateau of about 14°. Clause 3213. The composition of any one of clauses 1101 to 3120, wherein the formulation has a phase angle plateau of about 15°. Clause 3214. The composition of any one of clauses 1101 to 3120, wherein the formulation has a phase angle plateau of about 16°. Clause 3215. The composition of any one of clauses 1101 to 3120, wherein the formulation has a phase angle plateau of about 17°. Clause 3216. The composition of any one of clauses 1101 to 3120, wherein the formulation has a phase angle plateau of about 18°. Clause 3217. The composition of any one of clauses 1101 to 3120, wherein the formulation has a phase angle plateau of about 19°. Clause 3218. The composition of any one of clauses 1101 to 3120, wherein the formulation has a phase angle plateau of about 20°. Clause 3219. The composition of any one of clauses 1101 to 3120, wherein the formulation has a phase angle plateau of about 21°. Clause 3220. The composition of any one of clauses 1101 to 3120, wherein the formulation has a phase angle plateau of about 22°. Clause 3221. The composition of any one of clauses 1101 to 3120, wherein the formulation has a phase angle plateau of about 23°. Clause 3222. The composition of any one of clauses 1101 to 3120, wherein the formulation has a phase angle plateau of about 24°. Clause 3223. The composition of any one of clauses 1101 to 3120, wherein the formulation has a phase angle plateau of about 25°. Clause 3224. The composition of any one of clauses 1101 to 3120, wherein the formulation has a phase angle plateau of about 26°. Clause 3225. The composition of any one of clauses 1101 to 3120, wherein the formulation has a phase angle plateau of about 27°. Clause 3226. The composition of any one of clauses 1101 to 3120, wherein the formulation has a phase angle plateau of about 28°. Clause 3227. The composition of any one of clauses 1101 to 3120, wherein the formulation has a phase angle plateau of about 29°. Clause 3228. The composition of any one of clauses 1101 to 3120, wherein the formulation has a phase angle plateau of about 30°.

Clause 3300. The composition of any one of clauses 1101 to 3228, wherein the formulation has a yield stress between about 50 Pa and about 12,500 Pa. Clause 3301. The composition of any one of clauses 1101 to 3228, wherein the formulation has a yield stress between about 50 Pa and about 250 Pa. Clause 3302. The composition of any one of clauses 1101 to 3228, wherein the formulation has a yield stress between about 250 Pa and about 750 Pa. Clause 3303. The composition of any one of clauses 1101 to 3228, wherein the formulation has a yield stress between about 750 Pa and about 1,500 Pa. Clause 3304. The composition of any one of clauses 1101 to 3228, wherein the formulation has a yield stress between about 1,500 Pa and about 2,500 Pa. Clause 3305. The composition of any one of clauses 1101 to 3228, wherein the formulation has a yield stress between about 2,500 Pa and about 3,500 Pa. Clause 3306. The composition of any one of clauses 1101 to 3228, wherein the formulation has a yield stress between about 3,500 Pa and about 5,000 Pa. Clause 3307. The composition of any one of clauses 1101 to 3228, wherein the formulation has a yield stress between about 5,000 Pa and about 12,500 Pa. Clause 3308. The composition of any one of clauses 1101 to 3228, wherein the formulation has a yield stress between about 50 Pa and about 7,500 Pa. Clause 3309. The composition of any one of clauses 1101 to 3228, wherein the formulation has a yield stress between about 7,500 Pa and about 10,000 Pa. Clause 3310. The composition of any one of clauses 1101 to 3228, wherein the formulation has a yield stress between about 10,000 Pa and about 12,500 Pa.

Clause 3400. The composition of any one of clauses 1101 to 3310, wherein the formulation is aged between about 5 minutes and about 60 minutes before measuring any one of storage modulus, loss modulus, complex modulus, phase angle, and/or yield stress. Clause 3401. The composition of any one of clauses 1101 to 3310, wherein the formulation is aged between about 1 minute and about 5 minutes before measuring any one of storage modulus, loss modulus, complex modulus, phase angle, and/or yield stress. Clause 3402. The composition of any one of clauses 1101 to 3310, wherein the formulation is aged between about 5 minutes and about 10 minutes before measuring any one of storage modulus, loss modulus, complex modulus, phase angle, and/or yield stress. Clause 3403. The composition of any one of clauses 1101 to 3310, wherein the formulation is aged between about 10 minutes and about 15 minutes before measuring any one of storage modulus, loss modulus, complex modulus, phase angle, and/or yield stress. Clause 3404. The composition of any one of clauses 1101 to 3310, wherein the formulation is aged between about 15 minutes and about 20 minutes before measuring any one of storage modulus, loss modulus, complex modulus, phase angle, and/or yield stress. Clause 3405. The composition of any one of clauses 1101 to 3310, wherein the formulation is aged between about 20 minutes and about 25 minutes before measuring any one of storage modulus, loss modulus, complex modulus, phase angle, and/or yield stress. Clause 3406. The composition of any one of clauses 1101 to 3310, wherein the formulation is aged between about 25 minutes and about 30 minutes before measuring any one of storage modulus, loss modulus, complex modulus, phase angle, and/or yield stress. Clause 3407. The composition of any one of clauses 1101 to 3310, wherein the formulation is aged between about 30 minutes and about 35 minutes before measuring any one of storage modulus, loss modulus, complex modulus, phase angle, and/or yield stress. Clause 3408. The composition of any one of clauses 1101 to 3310, wherein the formulation is aged between about 35 minutes and about 40 minutes before measuring any one of storage modulus, loss modulus, complex modulus, phase angle, and/or yield stress. Clause 3409. The composition of any one of clauses 1101 to 3310, wherein the formulation is aged between about 40 minutes and about 45 minutes before measuring any one of storage modulus, loss modulus, complex modulus, phase angle, and/or yield stress. Clause 3410. The composition of any one of clauses 1101 to 3310, wherein the formulation is aged between about 45 minutes and about 50 minutes before measuring any one of storage modulus, loss modulus, complex modulus, phase angle, and/or yield stress. Clause 3411. The composition of any one of clauses 1101 to 3310, wherein the formulation is aged between about 50 minutes and about 55 minutes before measuring any one of storage modulus, loss modulus, complex modulus, phase angle, and/or yield stress. Clause 3412. The composition of any one of clauses 1101 to 3310, wherein the formulation is aged between about 55 minutes and about 60 minutes before measuring any one of storage modulus, loss modulus, complex modulus, phase angle, and/or yield stress.

Clause 3500. The composition of any one of clauses 1101 to 3412, wherein the fluid is phosphate-buffered saline (PBS).

Clause 3600. The composition of any one of clauses 1101 to 3500, wherein the tendon tissue is of human or animal origin.

Clause 4000. The composition of any one of clauses 1101 to 3600, wherein the composition is made by a method comprising a decellularizing step comprising contacting the tendon tissue with a DNase solution.

Clause 4010. The composition of clause 4000, wherein the method further comprises contacting a decellularized tendon tissue with a metalloproteinase (MMP) solution.

Clause 4020. The composition of clause 4000 or clause 4010, wherein the method further comprises a lyophilizing step.

Clause 4030. The composition of any one of clauses 4000 to 4020, wherein the method further comprises a reconstituting step.

Clause 4040. The composition of any one of clauses 4000 to 4030, wherein the method further comprises a washing step.

Clause 4050. The composition of any one of clauses 4000 to 4040, wherein the method further comprises a filtering step.

Clause 4060. The composition of any one of clauses 1101 to 4050, wherein the MMP comprises a collagenase.

Clause 5000. A method of modulating the storage modulus, loss modulus, and/or complex modulus of the formulation comprising the composition of any one of clauses 1101 to 4060, the method comprising aging the formulation for a period of time.

Clause 5100. The method of clause 5000, wherein the period of time is about 30 minutes. Clause 5101. The method of clause 5000, wherein the period of time is between about 5 minutes and about 10 minutes. Clause 5102. The method of clause 5000, wherein the period of time is between about 10 minutes and about 15 minutes. Clause 5103. The method of clause 5000, wherein the period of time is between about 15 minutes and about 20 minutes. Clause 5104. The method of clause 5000, wherein the period of time is between about 20 minutes and about 25 minutes. Clause 5105. The method of clause 5000, wherein the period of time is between about 25 minutes and about 30 minutes. Clause 5106. The method of clause 5000, wherein the period of time is between about 30 minutes and about 35 minutes. Clause 5107. The method of clause 5000, wherein the period of time is between about 35 minutes and about 40 minutes. Clause 5108. The method of clause 5000, wherein the period of time is between about 40 minutes and about 45 minutes. Clause 5109. The method of clause 5000, wherein the period of time is between about 45 minutes and about 50 minutes. Clause 5110. The method of clause 5000, wherein the period of time is between about 50 minutes and about 55 minutes. Clause 5111. The method of clause 5000, wherein the period of time is between about 55 minutes and about 60 minutes. Clause 5112. The method of clause 5000, wherein the period of time is between about 60 minutes and about 120 minutes.

Clause 5200. The method of clause 5000 or clause 5100, wherein the modulation of the storage modulus, loss modulus, and/or complex modulus is within one order of magnitude. Clause 5201. The method of clause 5000 or clause 5100, wherein the modulation of the storage modulus, loss modulus, and/or complex modulus is within 0.5 order of magnitude. Clause 5202. The method of clause 5000 or clause 5100, wherein the modulation of the storage modulus, loss modulus, and/or complex modulus is within 0.6 order of magnitude. Clause 5203. The method of clause 5000 or clause 5100, wherein the modulation of the storage modulus, loss modulus, and/or complex modulus is within 0.7 order of magnitude. Clause 5204. The method of clause 5000 or clause 5100, wherein the modulation of the storage modulus, loss modulus, and/or complex modulus is within 0.8 order of magnitude. Clause 5205. The method of clause 5000 or clause 5100, wherein the modulation of the storage modulus, loss modulus, and/or complex modulus is within 0.9 order of magnitude. Clause 5206. The method of clause 5000 or clause 5100, wherein the modulation of the storage modulus, loss modulus, and/or complex modulus is within 1 order of magnitude. Clause 5207. The method of clause 5000 or clause 5100, wherein the modulation of the storage modulus, loss modulus, and/or complex modulus is within 1.1 order of magnitude. Clause 5208. The method of clause 5000 or clause 5100, wherein the modulation of the storage modulus, loss modulus, and/or complex modulus is within 1.2 order of magnitude. Clause 5209. The method of clause 5000 or clause 5100, wherein the modulation of the storage modulus, loss modulus, and/or complex modulus is within 1.3 order of magnitude. Clause 5210. The method of clause 5000 or clause 5100, wherein the modulation of the storage modulus, loss modulus, and/or complex modulus is within 1.4 order of magnitude. Clause 5211. The method of clause 5000 or clause 5100, wherein the modulation of the storage modulus, loss modulus, and/or complex modulus is within 1.5 order of magnitude.

Clause 6000. A method of stimulating cartilage regeneration, bone regeneration, or tendon enthesis regeneration in a subject in need thereof, the method comprising administering to the subject the composition of any one of clauses 1101 to 5211 or any formulation thereof.

Clause 6100. A method of stimulating angiogenesis in a cartilage tissue, a bone tissue, or enthesis tissue in a subject in need thereof, the method comprising administering to the subject the composition of any one of clauses 1101 to 5211 or any formulation thereof.

Clause 6200. The method of clause 6000 or clause 6100, wherein the formulation comprises a lyophilized form of the composition and a fluid.

Clause 6300. The method of claim 6200, wherein the ratio of fluid to the composition is between about 1 ml and about 7 ml of fluid to about 1 g of composition. Clause 6301. The method of claim 6200, wherein the ratio of fluid to the composition is between about 0.5 ml and about 1 ml of fluid to about 1 g of composition. Clause 6302. The method of claim 6200, wherein the ratio of fluid to the composition is between about 1 ml and about 1.5 ml of fluid to about 1 g of composition. Clause 6303. The method of claim 6200, wherein the ratio of fluid to the composition is between about 1.5 ml and about 2 ml of fluid to about 1 g of composition. Clause 6304. The method of claim 6200, wherein the ratio of fluid to the composition is between about 2 ml and about 2.5 ml of fluid to about 1 g of composition. Clause 6305. The method of claim 6200, wherein the ratio of fluid to the composition is between about 2.5 ml and about 3 ml of fluid to about 1 g of composition. Clause 6306. The method of claim 6200, wherein the ratio of fluid to the composition is between about 3 ml and about 3.5 ml of fluid to about 1 g of composition. Clause 6307. The method of claim 6200, wherein the ratio of fluid to the composition is between about 3.5 ml and about 4 ml of fluid to about 1 g of composition. Clause 6308. The method of claim 6200, wherein the ratio of fluid to the composition is between about 4 ml and about 4.5 ml of fluid to about 1 g of composition. Clause 6309. The method of claim 6200, wherein the ratio of fluid to the composition is between about 4.5 ml and about 5 ml of fluid to about 1 g of composition. Clause 6310. The method of claim 6200, wherein the ratio of fluid to the composition is between about 5 ml and about 5.5 ml of fluid to about 1 g of composition. Clause 6311. The method of claim 6200, wherein the ratio of fluid to the composition is between about 5.5 ml and about 6 ml of fluid to about 1 g of composition. Clause 6312. The method of claim 6200, wherein the ratio of fluid to the composition is between about 6 ml and about 6.5 ml of fluid to about 1 g of composition. Clause 6313. The method of claim 6200, wherein the ratio of fluid to the composition is between about 6.5 ml and about 7 ml of fluid to about 1 g of composition. Clause 6314. The method of claim 6200, wherein the ratio of fluid to the composition is between about 7 ml and about 7.5 ml of fluid to about 1 g of composition. Clause 6315. The method of claim 6200, wherein the ratio of fluid to the composition is between about 7.5 ml and about 8 ml of fluid to about 1 g of composition. Clause 6316. The method of claim 6200, wherein the ratio of fluid to the composition is between about 8 ml and about 8.5 ml of fluid to about 1 g of composition. Clause 6317. The method of claim 6200, wherein the ratio of fluid to the composition is between about 8.5 ml and about 9 ml of fluid to about 1 g of composition. Clause 6318. The method of claim 6200, wherein the ratio of fluid to the composition is between about 9 ml and about 9.5 ml of fluid to about 1 g of composition. Clause 6319. The method of claim 6200, wherein the ratio of fluid to the composition is between about 9.5 ml and about 10 ml of fluid to about 1 g of composition.

Clause 6400. The method of any one of clauses 6200 or clause 6319, wherein the fluid is phosphate-buffered saline (PBS).

Clause 12001. A decellularized tendon matrix (DTM) composition comprising matrix metalloproteinase (MMP) digested tendon tissue.

Clause 12002. The decellularized tendon matrix (DTM) composition of clause 12001, further comprising an antimicrobial agent.

Clause 12003. The decellularized tendon matrix (DTM) composition of clause 12001, further comprising a sterile aqueous carrier solution.

Clause 12004. The decellularized tendon matrix (DTM) of any one of clauses 12001-12003, wherein the DTM is protein rich retains at least 50% of the growth factors present in the minced tendon tissue.

Clause 12005. A method of making a decellularized tendon matrix (DTM) composition, the method comprising one or more steps selected from: mincing a tendon tissue specimen; decellularizing the minced tendon tissue specimen; milling; digesting; stopping; neutralizing; washing; and lyophilizing.

Clause 12006. The method of clause 12005, wherein the digesting step comprises digesting with a matrix metalloproteinase (MMP) selected from the group consisting of MMP-2, MMP-9, MMP-14, and combinations thereof.

Clause 12007. A decellularized tendon matrix (DTM) composition wherein the DTM composition is prepared by a process comprising one or more steps selected from: mincing a tendon tissue specimen; decellularizing the minced tendon tissue specimen; digesting; and lyophilizing.

Clause 12008. A decellularized tendon matrix (DTM) composition wherein the DTM composition is prepared by a process comprising one or more steps selected from: mincing a tendon tissue specimen; decellularizing the minced tendon tissue specimen; milling; digesting; stopping and neutralizing; washing; and lyophilizing.

Clause 12009. The composition of any one clauses 12007 or 12008, wherein the decellularizing step comprises exposing the minced tendon tissue specimen to a solution comprising one or more components selected from the group consisting of a chaotropic salt, a non-ionic detergent, a zwitterionic detergent, a cationic detergent, an anionic detergent, or combinations thereof.

Clause 12010. The composition of any one of clauses 12007 or 12008, wherein the digesting step comprises digesting with a solution comprising a matrix metalloproteinase (MMP).

Clause 12011. The composition of clause 12010 wherein the matrix metalloproteinase (MMP) is selected from the group consisting of MMP-2, MMP-9, MMP-14, or combinations thereof.

Clause 12012. The composition of any one of clauses 12007 or 12008, wherein the stopping and neutralizing step comprises stopping and neutralizing with a solution comprising one or more protease inhibitor selected from the group consisting of TAPI-0, TAPI-1, TAPI-2, marimastat, phosphoramidon, luteolin, PMSF, pepstatin A, leupeptin, E-64, sodium orthovanadate, or combinations thereof.

Clause 12013. A method of stimulating tendon regeneration, the method comprising: (i) resuspending a DTM composition according to clause 12001 or 12007 in a pharmaceutically acceptable carrier; and (ii) applying the resuspended DTM composition to a tendon site in need of stimulating tendon regeneration.

Clause 12014. A decellularized tendon matrix (DTM) hydrogel, comprising a resuspended DTM composition according to clause 12001 or 12004, 1-ethyl-3-[3-dimethylaminopropyl]carbodiimide (EDC), and PEG-N-hydroxysuccinimide (NHS) ester.

Clause 12015. A soft-cast decellularized tendon matrix (DTM) object, wherein the soft-cast object is prepared by a process comprising one or more steps selected from: resuspending a decellularized tendon matrix (DTM) composition according to clause 12001 or 12004 in a physiological buffer; mixing the DTM composition with PEG-N-hydroxysuccinimide (NHS) ester to produce a soft hydrogel; transferring the soft hydrogel to a three dimensional mold; and curing and inactivating the polymerization reaction.

Clause 12016. A decellularized tendon matrix (DTM) hydrogel, comprising a resuspended DTM composition according to clause 12001 or 12007, further comprising 1-ethyl-3-[3-dimethylaminopropyl]carbodiimide (EDC) and a water-soluble coupling agent selected from N-hydroxysuccinimide (NHS) or a N-hydroxysulfosuccinimide (sulfoNHS) in conjunction with the (EDC) coupling agent.

Clause 12017. A method of treating a tendon tear and/or stimulating tendon regeneration in a subject, the method comprising: obtaining a decellularized tendon matrix (DTM) composition comprising matrix metalloproteinase (MMP) digested tendon tissue; resuspending the DTM composition in a pharmaceutically acceptable carrier; and applying the resuspended DTM composition to a tendon site in need of stimulating tendon regeneration.

Clause 12018. A decellularized tendon matrix produced from a native tendon, the decellularized tendon matrix comprising greater than 90% by weight of TGF-β in the native tendon.

Clause 12019. The decellularized tendon matrix of clause 12018, the decellularized tendon matrix comprising greater than 95% by weight of TGF-β in the native tendon.

Clause 12020. The decellularized tendon matrix of clause 12018, the decellularized tendon matrix comprising greater than 99% by weight of TGF-β in the native tendon.

Clause 12021. The decellularized tendon matrix of any one of clauses 12018 to 12020, comprising less than 5% by weight of cellular material in the native tendon.

Clause 12022. The decellularized tendon matrix of any one of clauses 12018 to 12020, comprising less than 2% by weight of cellular material in the native tendon.

Clause 12023. The decellularized tendon matrix of any one of clauses 12018 to 12020, comprising less than 1% by weight of cellular material in the native tendon.

Clause 12024. The decellularized tendon matrix of any one of clauses 12018 to 12020, comprising less than 0.1% by weight of cellular material in the native tendon.

Clause 12025. The decellularized tendon matrix of any one of clauses 12018 to 12024, wherein the decellularized tendon matrix is substantially free of TGF-β producing cells.

Clause 12026. The decellularized tendon matrix of any one of clauses 12018 to 12025, comprising less than 5% by weight of DNA in the native tendon.

Clause 12027. The decellularized tendon matrix of any one of clauses 12018 to 12025, comprising less than 2% by weight of DNA in the native tendon.

Clause 12028. The decellularized tendon matrix of any one of clauses 12018 to 12025, comprising less than 1% by weight of DNA in the native tendon.

Clause 12029. The decellularized tendon matrix of any one of clauses 12018 to 12025, comprising less than 0.1% by weight of DNA in the native tendon.

Clause 12030. The decellularized tendon matrix of any one of clauses 12018 to 12025, wherein the decellularized tendon matrix is substantially free of DNA.

Clause 12031. A method of producing a decellularized tendon matrix (DTM) composition from a tendon, the method comprising: decellularizing the tendon thereby producing a decellularized tendon; contacting the decellularized tendon with an enzymatic solution comprising a matrix metalloproteinase (MMP) to produce a digested, decellularized tendon; lyophilizing the digested, decellularized tendon to produce a lyophilized tendon; and reconstituting the lyophilized tendon to produce a decellularized tendon matrix.

Clause 12032. The method of clause 12031, wherein the decellularizing comprises contacting the tendon with a DNase solution.

Clause 12033. The method of clause 12032, wherein the DNase solution comprises about 10 to about 100 Units of DNase per milliliter of solvent, about 25 to about 75 Units of DNase per milliliter of solvent, about 40 to about 60 Units of DNase per milliliter of solvent, about 40 to about 60 Units of DNase per milliliter of solvent, or about 50 Units of DNase per milliliter of solvent.

Clause 12034. The method of any one of clauses 12032 or 12033, wherein the decellularizing comprises contacting the tendon with between about 4 milliliters and about 50 milliliters of the DNase solution per 1 gram of tendon.

Clause 12035. The method of clause 12034, wherein the decellularizing comprises contacting the tendon with between about 5 milliliters and about 10 milliliters of the DNase solution per 1 gram of tendon.

Clause 12036. The method of clause 12034, wherein the decellularizing comprises contacting the tendon with between about 10 milliliters and about 50 milliliters of the DNase solution per 1 gram of tendon.

Clause 12037. The method of any one of clauses 12034 to 12036, wherein the contacting occurs for a period of about 1 hour, and optionally occurs on a shaker.

Clause 12038. The method of any one of clauses 12031 to 12037, wherein the decellularizing further comprises washing the tendon with phosphate buffered saline.

Clause 12039. The method of any one of clauses 12031 to 12038, wherein the decellularizing further comprises filtering the tendon.

Clause 12040. The method of any one of clauses 12031 to 12039, wherein the lyophilizing comprises freezing the digested, decellularized tendon at minus 80° C. for at least about 30 minutes.

Clause 12041. The method of clause 12039, wherein the tendon is filtered through a 70 micrometer strainer using centrifugation at between about 1500 G to about 2500 G for between about 1 minute and about 15 minutes.

Clause 12042. The method of any one of clauses 12031 to 12041, wherein the MMP comprises collagenase.

Clause 12043. The method of clause 12042, wherein the collagenase is selected from the group consisting of Collagenase Type I, Collagenase Type III, and a combination thereof.

Clause 12044. The method of clause 12043 comprising Collagenase Type I, wherein the concentration of the Collagenase Type I in the enzymatic solution is about 2 milligrams per milliliter.

Clause 12045. The method of clause 12043 comprising Collagenase Type III, wherein the concentration of the Collagenase Type III in the enzymatic solution is about 1 milligram per milliliter.

Clause 12046. The method of any one of clauses 12031 to 12045, wherein the decellularized tendon is contacted with between about 10 milliliters and about 50 milliliters of the enzymatic solution per 1 gram of tendon.

Clause 12047. The method of any one of clauses 12018 to 12045, wherein the decellularized tendon is contacted with between about 5 milliliters and about 10 milliliters of the enzymatic solution per 1 gram of tendon.

Clause 12048. The method of any one of clauses 12031 to 12047, wherein the decellularized tendon is contacted with the enzymatic solution for about 24 hours.

Clause 12049. The method of any one of clauses 12031 to 12047, wherein the decellularized tendon is contacted with the enzymatic solution for about 12 hours.

Clause 12050. The method of any one of clauses 12031 to 12049, wherein the decellularized tendon is contacted with the enzymatic solution at about 37° C.

Clause 12051. The method of any one of clauses 12031 to 12050, wherein the reconstituting comprises mixing between about 2 microliters and about 5 microliters of solvent with about 1 milligram of lyophilized tendon.

Clause 12052. A decellularized tendon matrix produced according to the method of any one of clauses 12031 to 12051.

Clause 12053. A decellularized tendon matrix for implantation into a subject produced according to the method of any one of clauses 12031 to 12051.

Clause 12054. A tissue regeneration scaffold for implantation into a patient comprising the decellularized tendon matrix material produced according to the method of any one of clauses 12031 to 12051.

Clause 12055. The decellularized tendon matrix material of any one of clauses 12018 to 12030 and/or 12052 to 12054, further comprising an excipient.

Clause 12056. A composition comprising (i) a decellularized tendon matrix (DTM) composition or DTM hydrogel, comprising a resuspended DTM composition of any of the preceding clauses, 1-ethyl-3-[3-dimethylaminopropyl]carbodiimide (EDC), and PEG-N-hydroxysuccinimide (NHS) ester and (ii) a decellularized or devitalized cartilage matrix (DCM) composition or DCM hydrogel.

Clause 12057. The composition of clause 12056, wherein the percent total of DTM composition or DTM hydrogel to percent total of DCM composition or DCM hydrogel is about 1:99 by weight, about 5:95 by weight, about 10:90 by weight, about 15:85 by weight, about 20:80 by weight, about 25:75 by weight, about 30:70 by weight, about 35:65 by weight, about 40:60 by weight, about 45:55 by weight, about 50:50 by weight, about 55:45 by weight, about 60:40 by weight, about 65:35 by weight, about 70:30 by weight, about 75:25 by weight, about 80:20 by weight, about 85:15 by weight, about 90:10 by weight, about 95:5 by weight, or about 99:1 by weight.

Clause 12058. The composition of clause 12056, wherein the percent total of DTM composition or DTM hydrogel to percent total of DCM composition or DCM hydrogel is between about 1:99 by weight and about 99:1 by weight.

Clause 12059. A method of stimulating cartilage regeneration, bone regeneration, or tendon enthesis regeneration in a subject in need thereof, the method comprising administering to the subject a composition of any of the preceding clauses.

Clause 12060. A method of stimulating angiogenesis in a cartilage tissue, a bone tissue, or enthesis tissue in a subject in need thereof, the method comprising administering to the subject a composition of any of the preceding clauses.

Although the present disclosure has been described in considerable detail with reference to various versions thereof, other versions are possible. Therefore, the spirit and scope of the appended claims should not be limited to the description of the preferred versions contained herein.

The reader's attention is directed to all papers and documents which are filed concurrently with this specification and which are open to public inspection with this specification, and the contents or all such papers and documents are incorporated by reference herein. Furthermore, all publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls. All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is only one example of a generic series of equivalent or similar features.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1 Preparation of Decellularized Tendon Matrix

A human cadaveric Achilles tendon is washed with phosphate buffered saline (PBS), pH 7.4, then the sheath, adipose and synovial tissue is removed from tendon tissue specimen. The tendon tissue specimen is then minced into pieces roughly 1 to 4 mm³ in size, then washed with phosphate-buffered saline (PBS).

The minced tendon pieces are immersed in decellularization solution, comprising 1% w/v sodium dodecyl sulfate (SDS), and moderately agitated. The minced material is carefully washed multiple exchanges of ultrapure water to remove residual SDS and cellular components.

The material is then flash frozen then milled yielding a heterogeneous material with a range of particle sizes. The resulting material is then resuspended in MMP digestion buffer. This suspension is incubated.

Stop solution is then added to halt MMP digestion; the buffer is then changed and neutralization solution. The material is then washed with multiple buffer exchanges of wash buffer, and then lyophilized. Decellularization is assayed by comparing SYTO Green 11 (nuclear) staining of native tendon starting material to the final DTM product. Decellularization is further confirmed using Hematoxylin & Eosin, 4′,6-diamidino-2-phenylindole (DAPI) staining, agarose gel electrophoresis, and quantification of remnant DNA. The DTM product is substantially free of nuclear staining. Remnant DNA is present at or below about 2 ng/mL.

MALDI-TOF mass spectrometry is used to demonstrate the presence of TGF-β in the DTM product.

### Example 2 Characterization of DTM Hydrogels

A DTM hydrogel is prepared by resuspending a DTM of the disclosure in a pharmaceutically acceptable sterile solution for injection. Then the following methods, according to Zuidema et al., J. Biomed. Mater. Res. B Appl. Biomater., 102:1063-73 (2014) are used to characterize the resulting DTM hydrogel: (1) Time sweep to determine the gelation time of the hydrogel. (2) Strain sweep to determine the linear-viscoelastic region of the hydrogel with respect to strain. (3) Frequency sweep to determine the linear equilibrium modulus plateau of the hydrogel. (4) Time sweep with values obtained from strain and frequency sweeps to accurately report the equilibrium moduli and gelation time.

### Example 3 DTM Processing for Maintaining a Native Growth Factor Profile

Decellularization and enzymatic processing techniques were developed to generate a decellularized tendon matrix putty that preserves TGF-β bioactivity in order to promote tissue regeneration.

Tendons have a poor regenerative capacity and typically heal through scarring rather than with a native-like tissue structure resulting in diminished mechanical strength. As a consequence, tendon repairs, such as rotator cuff repairs, have failure rates ranging from 20 to 90% depending on patient age, tear size and other biological factors. There is an unmet clinical need to stimulate tendon healing to produce a stronger regenerate in order to improve patient outcomes.

Decellularized extracellular matrix (ECM) have been frequently utilized as a regenerative material for tissue engineering as it retains proteins and growth factors native to the tissue and also can provide structural support. There are multiple growth factors which drive tendon remodeling, specifically transforming growth factor beta (TGF-β), has been studied for its role in regenerative healing. It has been shown that TGF-β signaling is critical in the formation of tendons during development. Following injury, TGF-β is temporally regulated to promote healing by stimulating collagen production and angiogenesis. Furthermore, exogenous TGF-β1 injections were reported to increase collagen type I and III mRNA and an increase in biomechanical function of the repaired tendons was also found in this group.

Objectives—(i) To develop a decellularization technique, (ii) To develop a method for enzymatically digesting decellularized tendons, and (iii) To characterize the protein profile of decellularized tendon matrix (DTM).

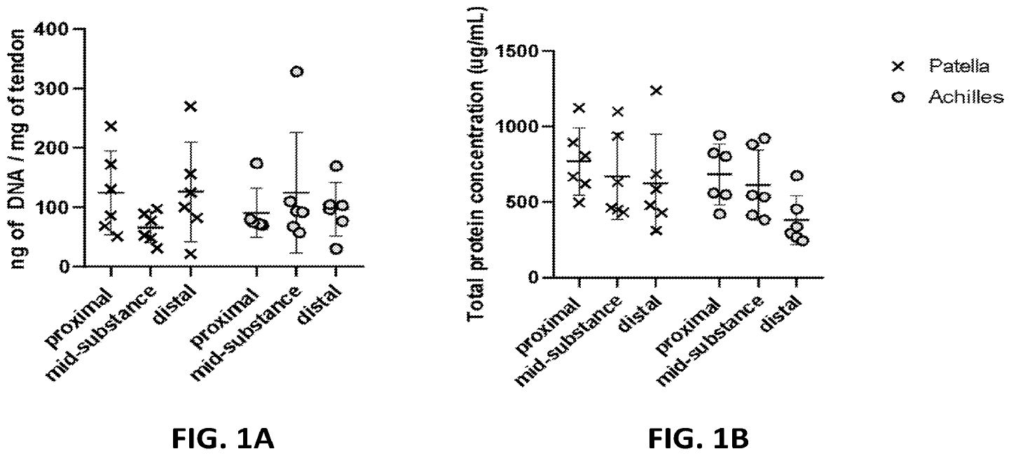

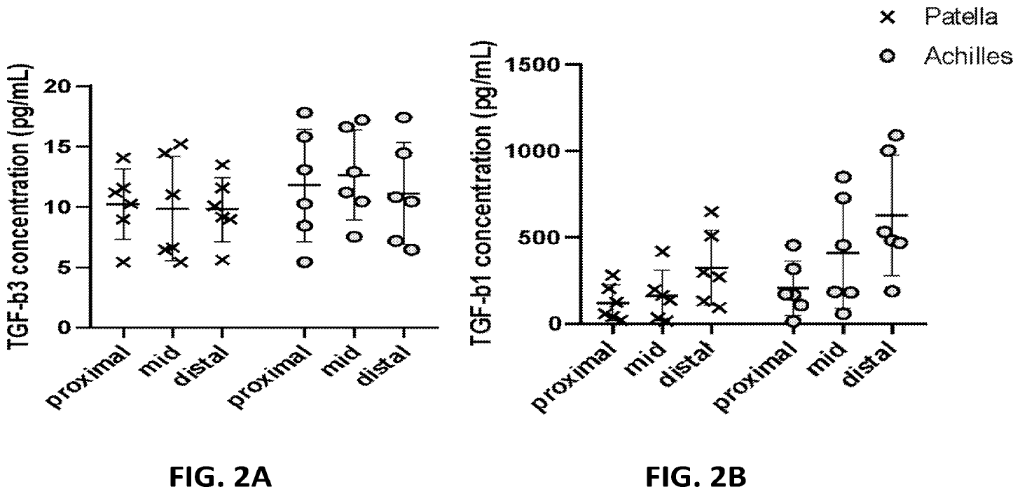

Native Tendon Characterization—The goal was to determine which tendons were best to develop an allograft product. Patella and Achilles tendons were characterized for DNA content and native protein concentrations. Any differences between location and protein profile within each source (i.e. proximal vs. distal) was also determined. As shown in FIGS. 1A-B and 2A-B, no significant difference between patella and Achilles tendons was found. DNA content was measured using DNEasy kits (Qiagen). Total protein content was measured using a BCA kit (Thermo Scientific). As TGF-β is a pivotal growth factor in tendon healing, it is important to determine preprocessed (native) TGF-β concentrations within each tendon (patella vs. Achilles) and its location (proximal, mid, distal) (see, e.g., FIGS. 2A-B).

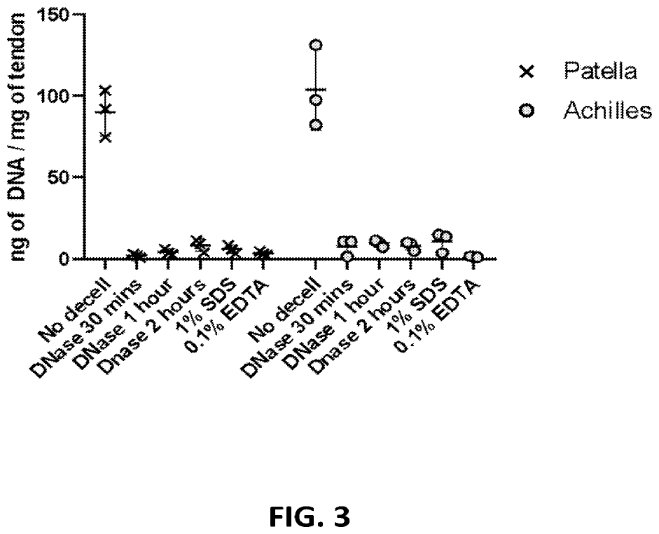

Detergent-free Decellularization—The aim of the study was to develop a gentler and faster method of decellularization compared to traditional detergent-based method. DNase was compared to detergents, such as SDS and EDTA, which often have long processing times (1-2 weeks). Different time and concentrations of DNase were tested. As shown in FIG. 3, it was determined that 1 hour of decell with DNase 50 U was significantly different than the native DNA content and was shown to be equivalent to traditional methods.

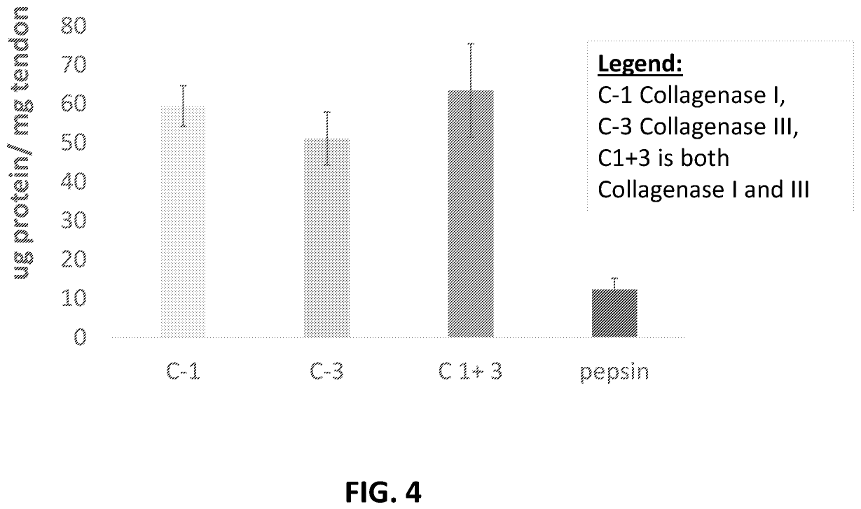

Collagenase Digestion Maximizes Protein Content—Enzymatic digestion allows for decellularized tendons to be manipulated into surgical friendly forms, such as an injectable system or a putty. Enzymatic digestion was modified in order to maximize functionality of growth factors. As shown in FIG. 4, collagenase I, III and a combination of the two were compared to pepsin digestion. All tendon samples were measured in μg total protein per mg tissue (μg protein/mg tissue). To make the enzymatic solutions, collagenase I (Life Technologies) was used at 2 mg/l mL PBS, collagenase III (MP Biomedicals) at 1 mg/l mL PBS and pepsin (Sigma) at 1 mg/l mL 0.1 M HCl. All samples were incubated for 24 hours.

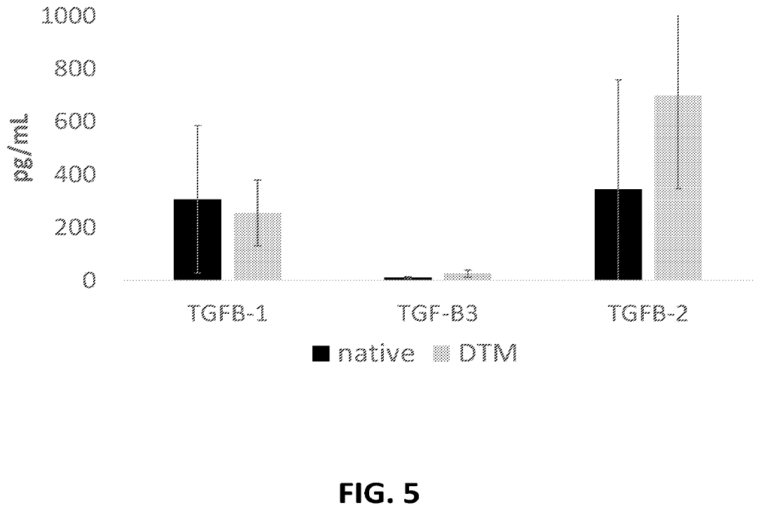

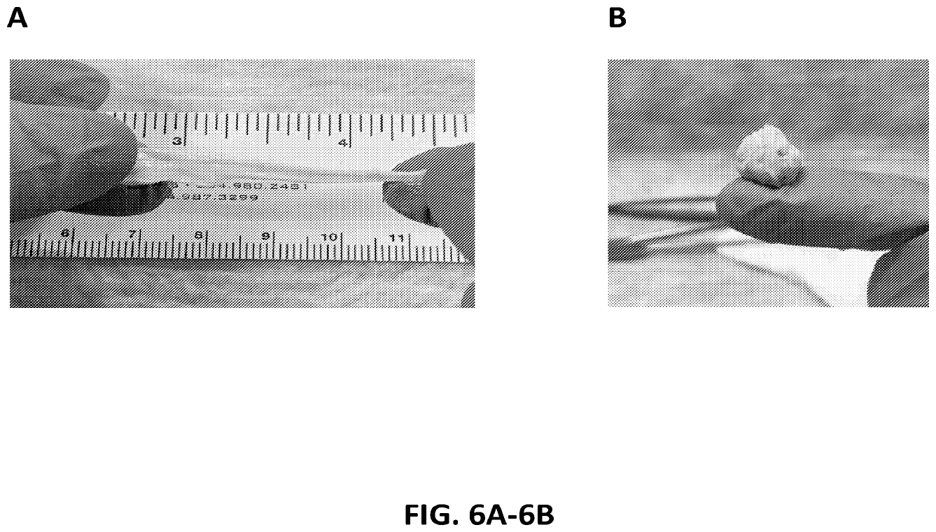

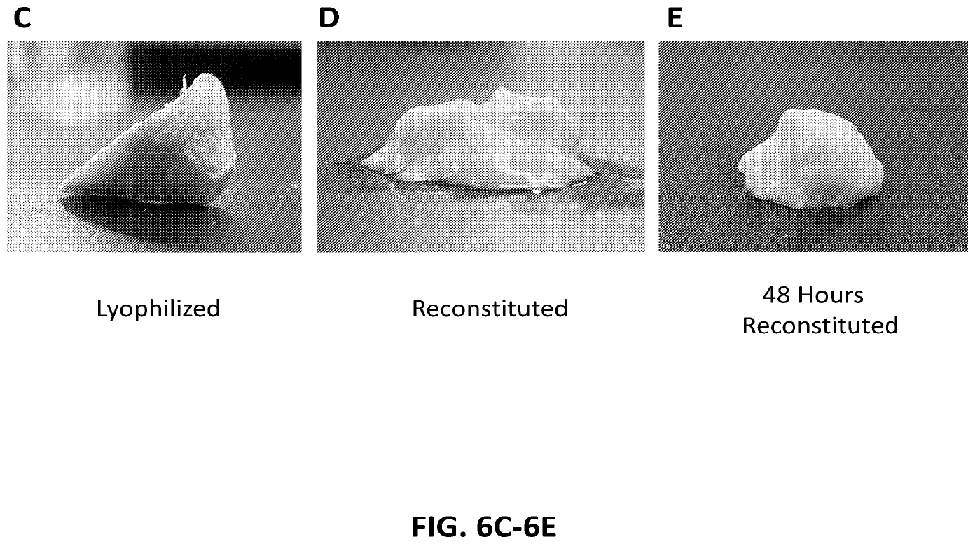

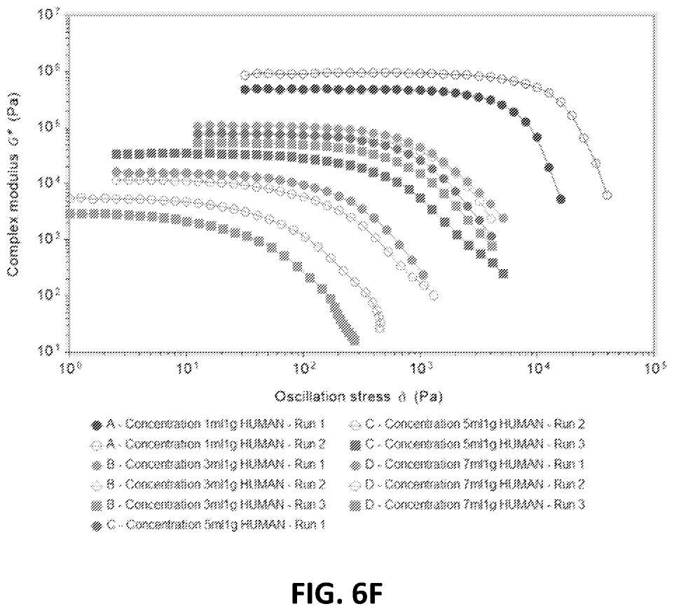

Decellularized Tendon Matrix (DTM) Maintains TGF-β Proteins—To ensure that our decellularized tendon matrix (DTM) maintained bioactivity, TGF-β levels of the native tissue were compared to the processed product. TGFβ I, II and III all play an important role in tendon healing and repair. Following enzymatic digestion and a final lyophilization step, 30 μg of total protein was measured per sample based on the BCA results. A TGF-β Milliplex kit (Millipore Sigma) was utilized for measurement of all DTM samples. As shown in FIG. 5, The final prototype of DTM retains TGFβ I, II and III. As shown in FIGS. 6A-B, DTM processing facilitates an elastic characteristic which has the capacity to stretch (FIG. 6A) without being pulled apart.

There is an unmet clinical need to stimulate tendon healing to produce a stronger regenerate in order to improve patient outcomes. Current standard of care in tendon repair has high failure rates due in part to excessive scarring leading to reduced biomechanical functionality of the joint. In this study, a technique to generate a decellularized tendon matrix putty that preserves TGF-β bioactivity in order to promote tissue regeneration has been developed. Additional testing is being done, such as in vitro assays focused on cellular response to the DTM and an in vivo rotator cuff repair model to further characterize DTM efficacy in promoting tendon repair.

### Example 4 Tendon Decellularization, and Enzymatic Digestion and Reconstitution of Decellularized Tendon Matrix (DTM)

The aim was to develop a gentler and faster method of decellularization compared to traditional detergent-based method. DNase was compared to detergents, such as SDS and EDTA, which often have long processing times (1-2 weeks). Different time and concentrations of DNase were tested. It was determined that 1 hour of decell with DNase 50 U was significantly different than the native DNA content and was shown to be equivalent to traditional methods. DTM was prepared according to the following procedure.

Method of Tendon Decellularization—First, the tendon is weighed and recorded. Next, the tendon is minced into homogenously sized, smaller pieces. Next, to decellularized, the minced pieces are placed in DNase solution (see, e.g., table below; at 0.5 g tendon/mL DNase solution; DNase solution: 50 U DNase I per 1 mL 1×PBS; for 2 gram minced tendon, place in 4 mL 1×PBS and add 200 U DNase). Next, incubate at 56° C. for 1 hour with moderate shaking. Next, to wash the DTM, add 1×PBS at twice the initial volume (if 1 mL DNase solution was added, add 2 mL of 1×PBS). Next, place the DTM on 70 um cell strainers and centrifuge at 2000 G for 5-10 mins. Finally, freeze at −80° C. for at least 30 minutes, and place the tube in lyophilizer.

| | Putty | Injectable |
|---|---|---|
| Broad Range | .02-.25 g | .02-.25 g |
| | tendon/mL | endon/mL |
| | collagenase | collagenase |
| | solution | solution |
| Optimal | .10-.20 g | .02-.1 g |
| Range | tendon/mL | tendon/ |
| | collagenase | collagenase |
| | solution | solution |

Enzymatic Digestion (Injectable DTM)—First, the decellularized tendon is weighed and recorded. Next, To create an injectable, weigh out 0.02-0.10 g tendon and add 1 mL collagenase solution (Collagenase type I @ 2 mg/mL, Collagenase type III @ 1 mg/mL in 1×PBS). Next, incubate at 37° C. for 24 hours. Next, to wash the DTM, add 1×PBS at twice the initial volume (if 1 mL collagenase solution was added, add 2 mL of 1×PBS). Next, place the DTM on 70 um cell strainers and centrifuge at 2000 G for 5-10 mins. Next, place the DTM into a new microcentrifuge tube with 1 mL of PBS, and vortex for 30 sec. Next, place this solution into a 100 KDa filter, and spin at 12,000 G for 5 mins. Finally, freeze at −80° C. for at least 30 minutes, and place the tube in lyophilizer.

Enzymatic Digestion (Putty DTM)—First, the decellularized tendon is weighed and recorded. Next, To create a putty, weigh out 0.10-0.20 g tendon and add 1 mL collagenase solution (Collagenase type I @ 2 mg/mL, Collagenase type III @ 1 mg/mL in 1×PBS). Next, incubate at 37° C. for 12 hours. Next, to wash the DTM, add 1×PBS at twice the initial volume (if 1 mL collagenase solution was added, add 2 mL of 1×PBS). Next, place the DTM on 70 um cell strainers and centrifuge at 2000 G for 5-10 mins. Next, place the DTM into a new microcentrifuge tube with 1 mL of PBS, and vortex for 30 sec. Next, place this solution into a 100 KDa filter, and spin at 12,000 G for 5 mins. Finally, freeze at −80° C. for at least 30 minutes, and lyophilize.

Reconstitution—Add 2-5 uL of 1×PBS/mg tendon, and add additional PBS can be added until you reach desired consistency.

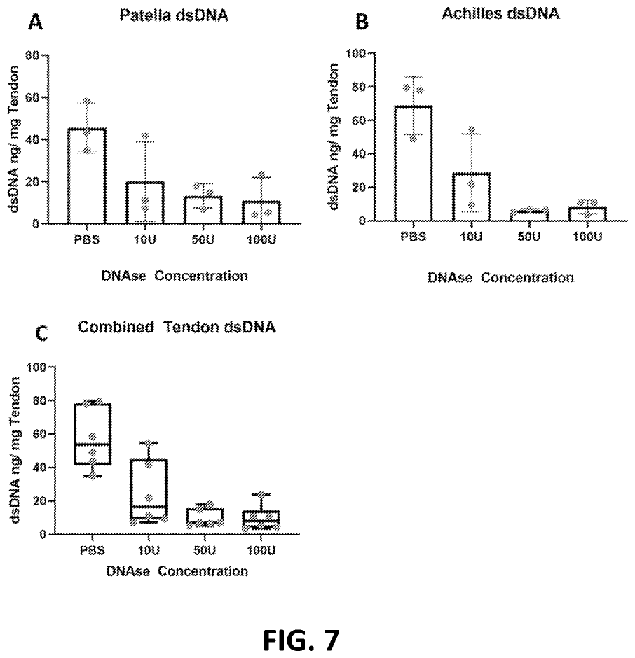

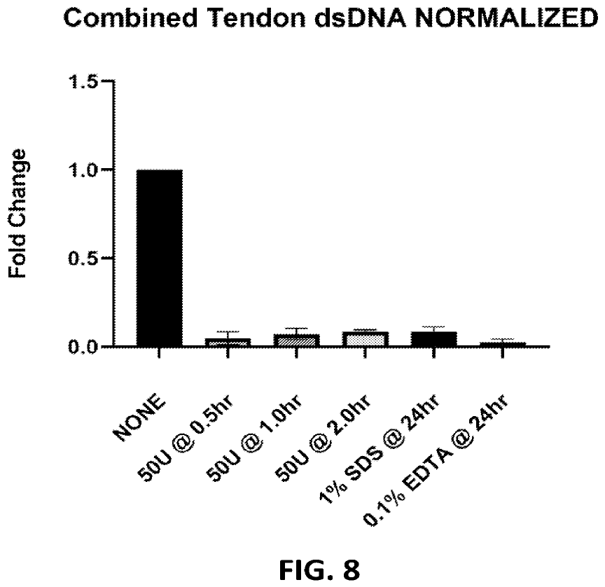

Tendon was decellularized using various concentrations of DNAse (10 U, 50 U, and 100 U) over 1 hour (see, e.g., FIG. 7). 1×PBS was used as a control for no decellularization. DNA concentration was determined using DNEasy kits (Qiagen). This data shows that as little as 50 U of DNAse is effective in decellularizing tissue. As shown in FIG. 8, DNAse at 50 U was compared to traditional detergents, 1% SDS and 0.1% EDTA. DNAse 50 U was tested at 0.5 hours, 1 hours, and 2 hours, while standard SDS and EDTA protocol calls for a 24-hour decellularization. DNA concentration was determined using DNEasy kits (Qiagen, n=3). All values were normalized to no decellularization. Tukey's HSD multiple comparison post-hoc testing shows no significant difference between the different times of DNAse treatment or decellularization by DNAse versus SDS and EDTA. Also shown in the following table, is the percent DNA left in Patella and Achilles tendons following various decell methods, and/or different time periods:

| | | DNAse | DNAse | DNAse | | |
|---|---|---|---|---|---|---|
| | no decell | 30 mins | 1 hr | 2 hr | 1% SDS | 0.1% EDTA |
| Avg % | 100.00% | 5.07% | 7.32% | 8.60% | 8.70% | 2.61% |
| DNA left | | | | | | |
| % Range | | 2-8% | 4-10% | 7-9% | 6-12% | 1-3% |
| DNA left | | | | | | |

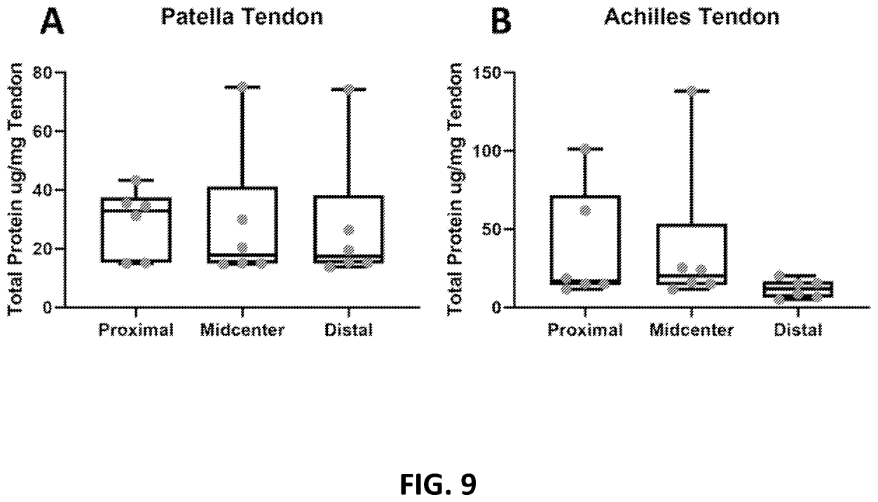

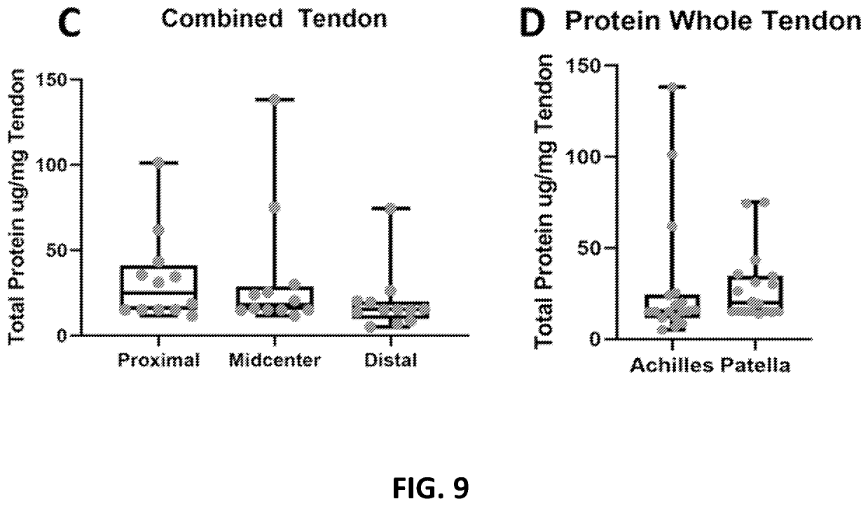

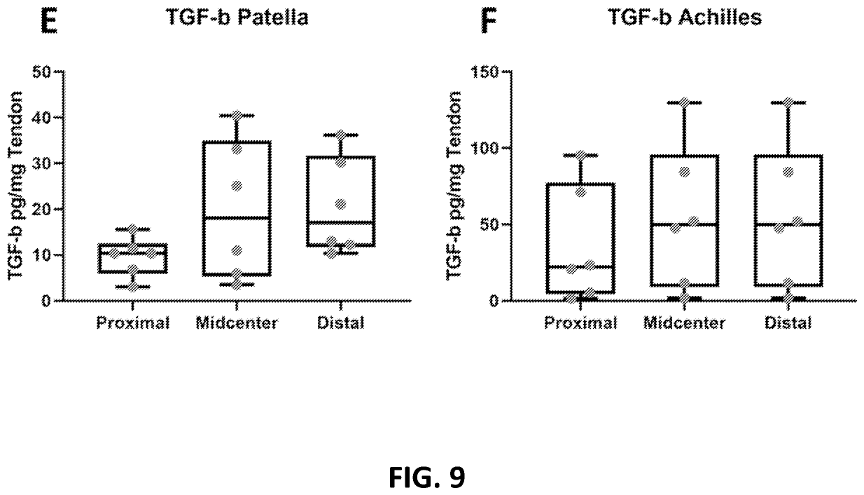

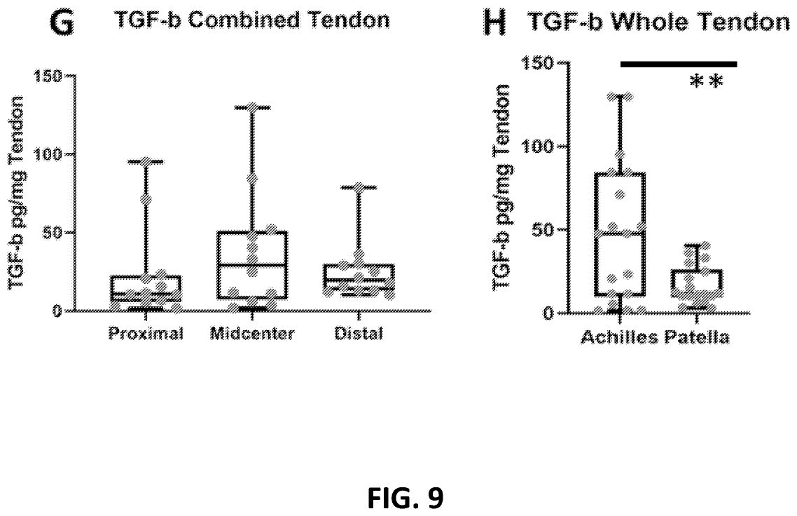

Native tendon was characterized to determine which tendons were best to develop an allograft product. Patella and Achilles tendons were characterized for DNA content and native protein concentrations. We also aimed to determine any difference between location and protein profile within each source (i.e. proximal vs. distal). No significant difference was found in total protein or TGFβ content between the different regions of the tendon. However, we did find that Achilles has a higher relative content of protein. The Achilles and Patellar tendons were divided into ⅓ sections consisting of the proximal, midcenter/middle, and distal ends of the tendon. (FIG. 9A-D) Total protein of the native tendons was measured using a BCA protein quantification kit (Thermo Scientific). (FIG. 9E-H) TGF-β was measured using a TGF-β magnetic bead panel Milliplex kit (Millipore Sigma, #TGFBMAG-64K-03). ANOVA shows no statistically significant differences between the regions of the tendons and therefore the entirety of the tendon can used through processing. When comparing the two different tendons (FIG. 9D) total protein is not different (P=0.93), but (FIG. 9H) TGF-β is statistically higher in Achilles than Patellar tendon (P=0.0045).

| | F Value | P Value | Significant |
|---|---|---|---|
| 9A | F (2, 15) = 0.01075 | 0.9893 | No |
| 9B | F (2, 15) = 1.069 | 0.3680 | No |
| 9C | F (2, 33) = 0.9342 | 0.4030 | No |
| 9E | F (2, 15) = 1.849 | 0.1915 | No |
| 9F | F (2, 15) = 0.3373 | 0.7190 | No |
| 9G | F (2, 33) = 0.7912 | 0.4617 | No |

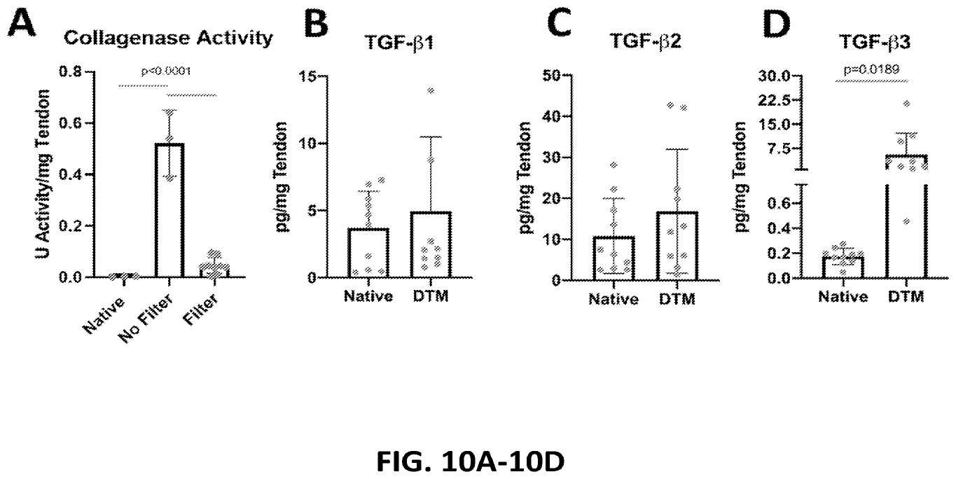

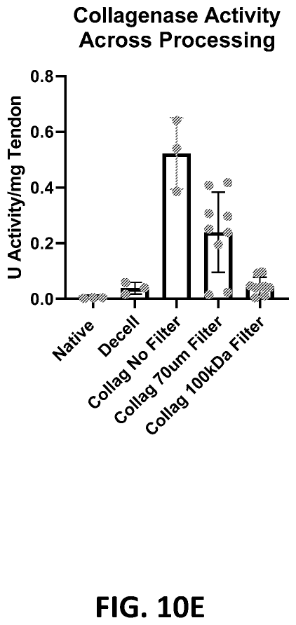

As shown in FIG. 10E, filtering effectively eliminated collagenase activity. Decellularized tendon was treated with collagenase to improve form-factor of DTM. 100 kDa filters were highly effective in eliminating the collagenase activity in the final product. ANOVA indicates that the groups have significant differences (F (4, 22)=18.06, p<0.0001). Importantly, there are no significant differences in collagenase activity between native and 100 kDa filtered samples.

| Comparison | p Value | Significant |
|---|---|---|
| Native vs. Decellularized | 0.9919 | No |
| Native vs. Collagenase No Filter | <0.0001 | **** |
| Native vs. Collagenase 70 um Filter | 0.0116 | * |
| Native vs. Collagenase 100 kDa Filter | 0.9635 | No |
| Decellularized vs. Collagenase No Filter | <0.0001 | **** |
| Decellularized vs. Collagenase 70 um | 0.0381 | * |
| Filter | | |
| Decellularized vs. Collagenase 100 kDa | >0.9999 | No |
| Filter | | |
| Collagenase No Filter vs. Collagenase 70 | 0.0021 | ** |
| um Filter | | |
| Collagenase No Filter vs. Collagenase | <0.0001 | **** |
| 100 kDa Filter | | |
| Collagenase 70 um Filter vs. Collagenase | 0.003 | ** |
| 100 kDa Filter | | |

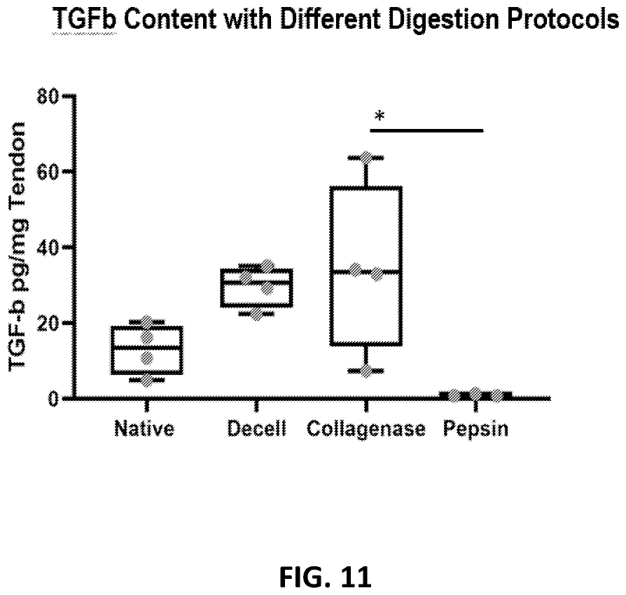

As shown in FIG. 11, more bioactivity is retained in DTM than standard methods for decellularizing tendon with pepsin. Tendons were digested following decellularization, using a solution containing Collagenase Type 1 (92.5 g tendon/g Collagenase 1) and 3 (185 g tendon/1 g Col 3), or using Pepsin given previous published methodologies (Farnebo et. al 2014, PMID: 24341855). ANOVA indicated significant differences between groups, F (3,11)=5.056, p=0.0193. Tukey's HSD post hoc shows pepsin has significantly less TGF-b (P=0.0249).

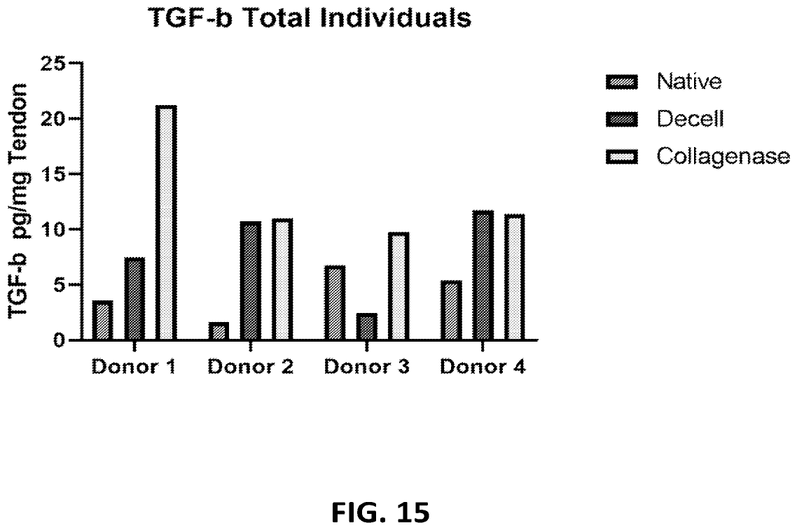

As shown in FIG. 15, the normalized TGFb content across four samples from four different donors, over the two processing steps. For each respective donor, the first column represents the amount of TGFb in the native tendon, the second column represents the amount of TGFb in the decellularized tendon, and the third column represents the amount of TGFb in the digested tendon. The percent changes across the processing steps is also described in the following table (percent increase is measured from native tendon to post collagenase processing):

| Donor | % Increase in TGF-b |
|---|---|
| #1 | 590.16 |
| #2 | 677.04 |
| #3 | 144.75 |
| #4 | 210.36 |

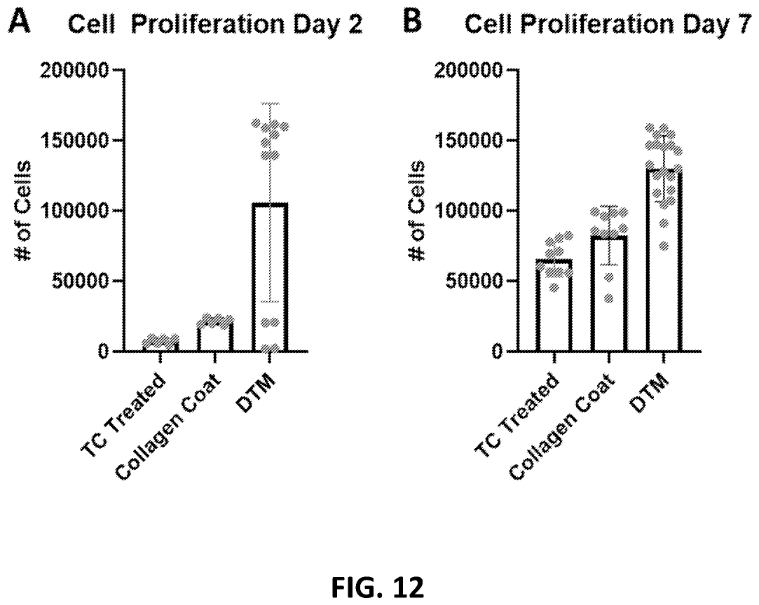

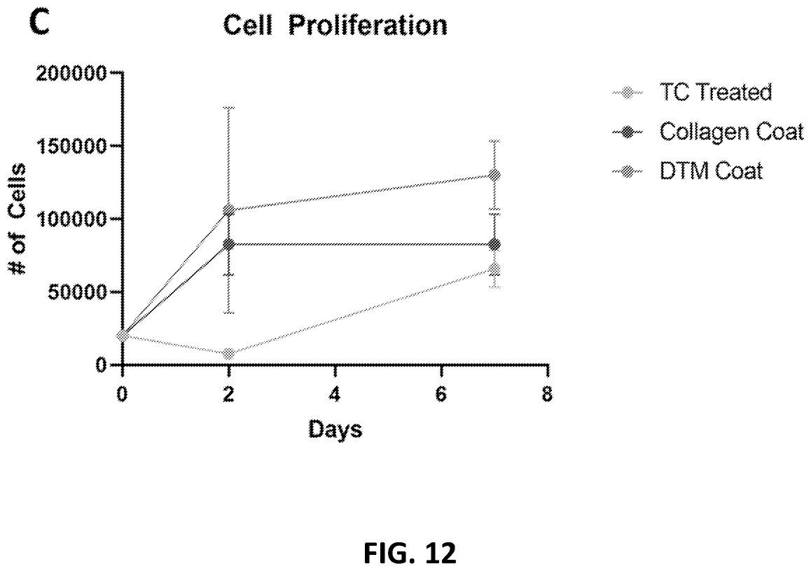

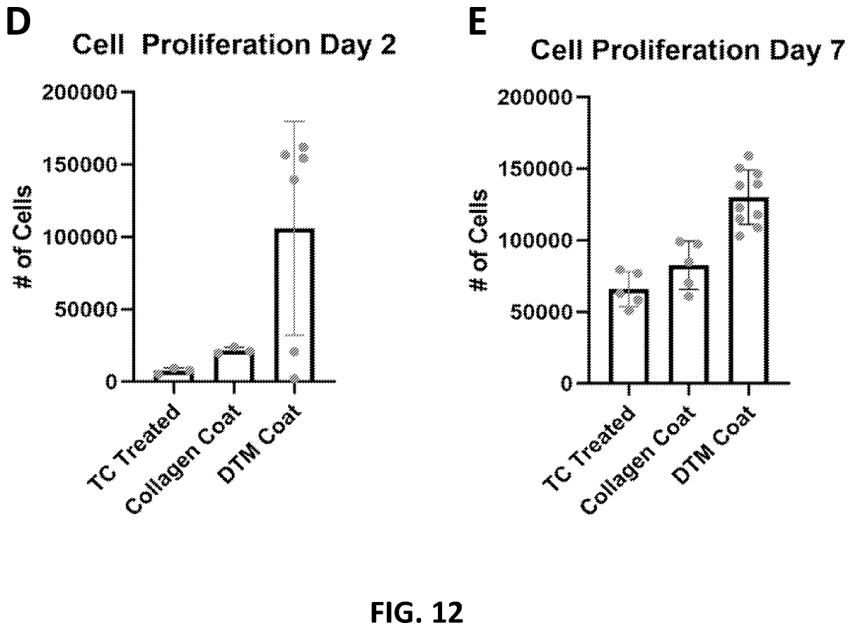

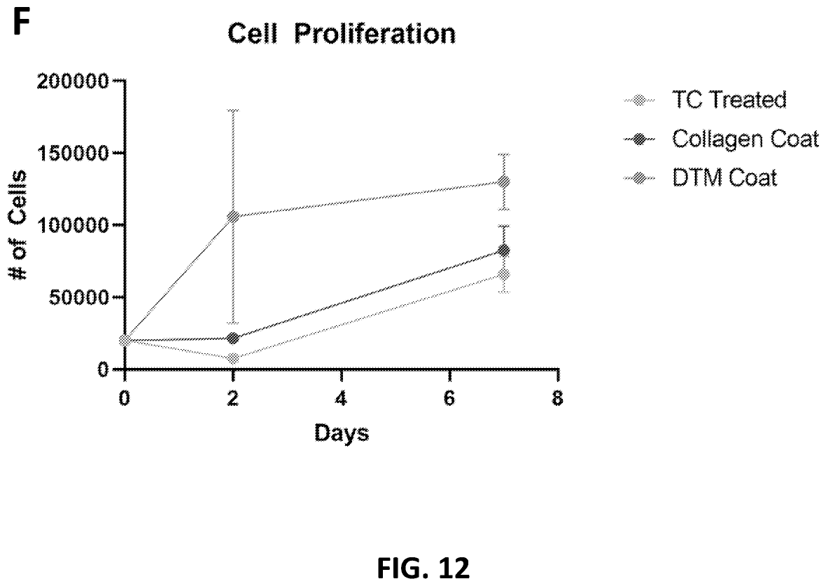

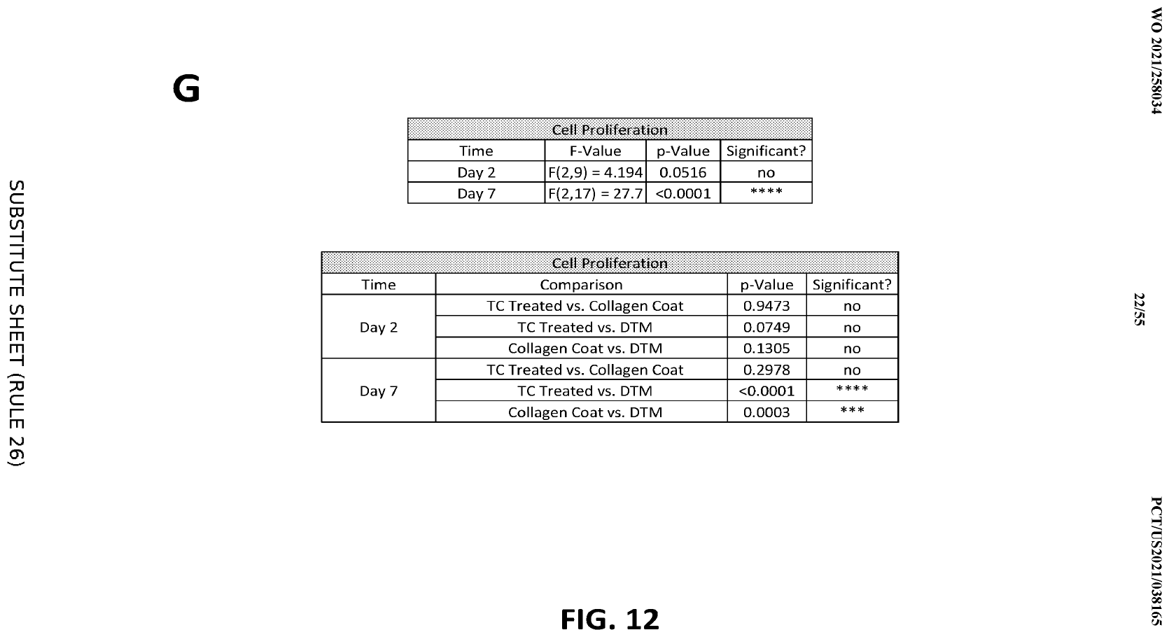

Differences in proliferation of cells plated on different surfaces was investigated (see, e.g., FIGS. 12A-C). Tissue culture plates were left untreated (control, “TC treated”), coated with collagen or with the DTM. Primary tenocytes (ZenBio #TEN-F) were plated at 20,000 cells/well and cell viability quantified using the Presto Blue (Thermo Fisher) at (A) 48 hours or (B) 7 days after plating, generating significantly different growth rates (C). (ANOVA=F (3,26)=10.6, p<0.0001).

| | Comparison | p Value | Significant |
|---|---|---|---|
| Day 2 | TC Treated vs. Collagen | 0.8816 | No |
| | Coat | | |
| | TC Treated vs. DTM | 0.0025 | ** |
| | Collagen Coat vs. DTM | 0.0089 | ** |
| Day 7 | TC Treated vs. Collagen | 0.1792 | No |
| | Coat | | |
| | TC Treated vs. DTM | <0.0001 | *** |
| | Collagen Coat vs. DTM | <0.0001 | *** |

### Example 5 Preservation of TGFβ in an Engineered Decellularized Tendon Matrix Putty Promotes Regeneration

Material and Methods

Tendon Allograft

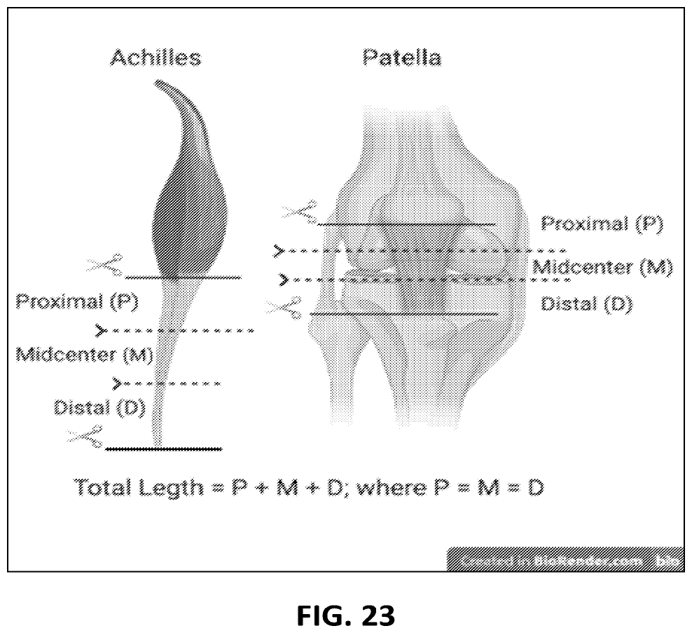

Achilles and patella tendon allografts were donated from Musculoskeletal Transplant Foundation (MTF, Edison, N.J.) using the MTF Biologics Non-Transplantable Tissue Program. A total of 10 donors (5 males and 5 females with ages ranging from 18 to 61) were used in this study. Tendons were delivered on dry ice, thawed, and then dissected into proximal, mid, and distal thirds for regional characterization. (FIG. 23) Dissected tendon was then finely minced and stored at −80° C. until ready for analysis; precaution was taken to insure only a single freeze-thaw cycle was done on all tendon tissues.

Tendon Decellularization

The tendon was decellularized using a proprietary tissue processing method (DTM) that involves application of a highly specific enzymatic decellularization solution for 1 hour followed by a wash step. DTM processing was compared to standard decellularization detergents sodium dodecyl sulfate (SDS, 1%) and ethylenediaminetetraacetic acid (EDTA, 0.1%) according to a previously published protocol. All decellularization solutions were diluted in 1× phosphate buffered sodium (PBS), and the untreated tendon (control) was incubated in only IX PBS. DNA was isolated using DNeasy Blood & Tissue Kit (Qiagen, Cat #69506) per manufacturer's protocol. The DNA isolate was measured with Tecan's Nano Quant Plate analyzed using the 260/280 ratio read by Tecan's Infinite 200 Pro Plate reader (Cat #30050303).

Protein Isolation and Quantification

To obtain protein isolates, T-PER (Thermo Fisher, Cat #78510) was added to the tendon samples with Protease Inhibitor Cocktail (1×) (Cell Signaling, Cat #1861278). The samples were homogenized using a tissue homogenizer (IKA, Cat #UX-04720-51) and placed at 4° C. for 2 hours to allow for protein extraction. The samples were purified by filtered through 70 μm cell strainers and spinning at 12,000×g for 10 minutes. Total protein was quantified using a disulfide reducing agent compatible microplate bicinchoninic acid assay (Micro-BCA, Thermo Fisher, Cat #23252) according to manufacturer's protocol using bovine serum albumin to generate the standard curve. Protein concentrations of the experimental values were calculated using a linear model in micrograms of protein per milliliter of diluted sample. The protein concentration per milligram of tendon was based on the initial dry weight of the sample used to make the protein isolate, which was measured using a Mettler Toledo New Classic ML milligram scale (Cat #ML104).

TGFβ Protein Analysis

Protein isolates were used to quantify TGFβ using Milliplex Map TGFβ Magnetic Bead 3 Plex Kit (Millipore Sigma, #TGFBMAG-64K-03). A Luminex 200™ Instrument System (Luminex, Cat #LX200-XPON-IVD) was used to detect the analytes according to the Bead Panel's manufacturer protocol. Data was imported into Milliplex Analyst Software to calculate pg/mL. Thirty (30) mg of total protein was placed within each of the wells for TGFβ analysis, and the final output is expressed in pg of TGFβ per mL of sample. To get the concentration of TGFβ per mg of the tendon, output values were multiplied by their dilution factor and related back to the initial weight of the sample used to make the protein isolate.

Tendon Digestion

To generate a tendon putty, a proprietary process was developed that involved mechanical and enzymatic processing, filtration, and wash steps. Inactivation of the enzymatic digestion was verified using a Collagenase Activity Colorimetric Assay Kit (Abcam, Cat #ab196999). Isolates were compared to a positive control of collagenase with an activity of 0.35 U/mL, as well as a negative control comprised of collagenase and 1,10-Phenanthroline as an inhibitor. The reaction plate was quantified using Tecan's Infinite 200 Pro plate reader at an absorbance of 345 nm. Activity was calculated given manufacturer protocol using the equation:

$$Collagenase = \frac{( \frac{Δ ODc}{Δ T} ) \times 0.2 \times D}{0.53 \times V}$$

### Where:

ΔOD=change in absorbance from T1 to T2, corrected for background
ΔT=change in time between readings in minutes
0.2=reaction volume
D=samples dilution factor
0.53=millimolar extinction coefficient of FALGPA
V=sample volume added into the reaction well in mL
Activity was related back to total protein of the sample and expressed in U/mg of the tendon.

Cell Proliferation Assay

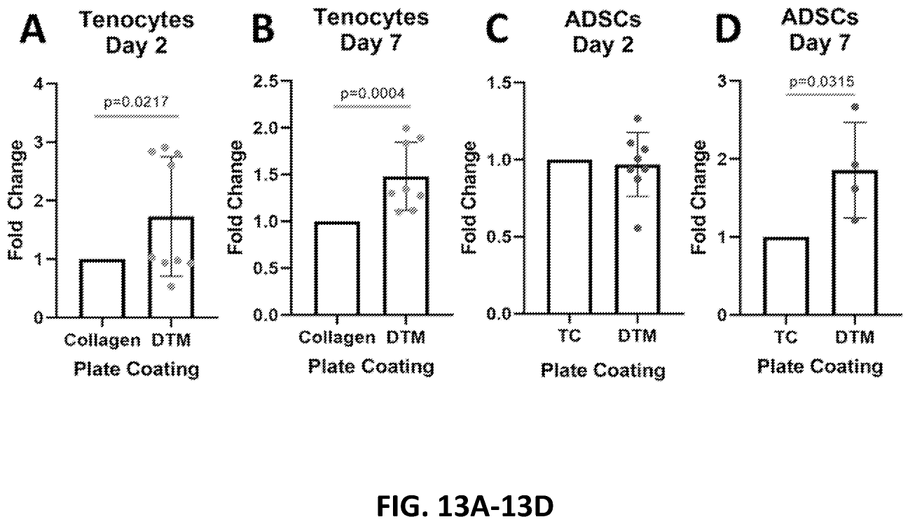

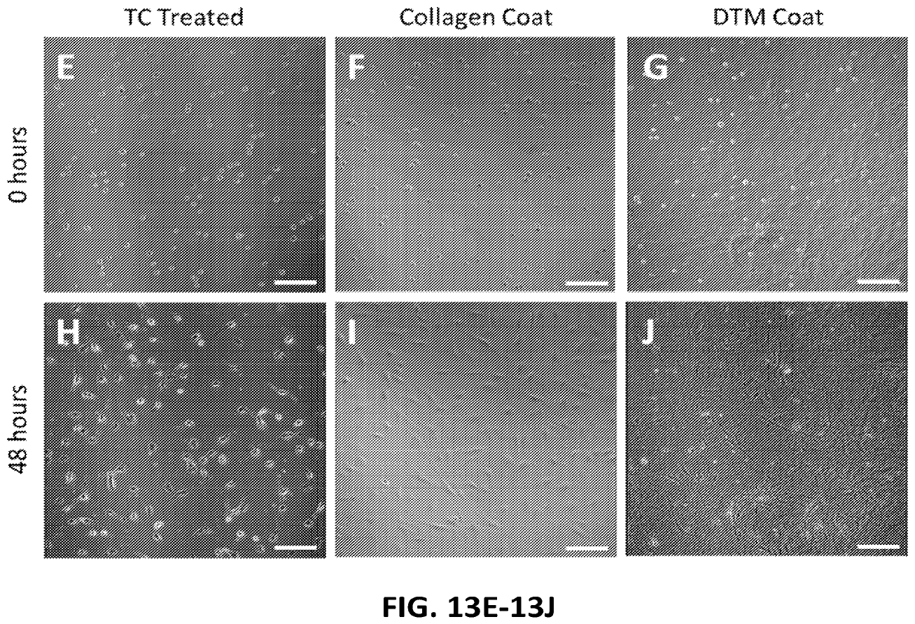

Lyophilized tendons digested according to the DTM protocol were resuspended at a concentration of 0.03 g/l mL in 1×PBS and added to 24 well tissue culture plates. Collagen coating was done using a collagen type 1 (Sigma, Cat #C-9791) solution at a concentration of 0.1 mg/l mL in aqueous acetic acid. Plates were left to dry for 4 hours in a ventilated biosafety cabinet before the solution was removed. Plates continued to dry overnight, with UV sterilization for 5 minutes. Tenocytes were purchased from (ZenBio, Cat #TEN-F) or adipose-derived stem cells (ADSCs) donated from CellTex were plated onto the coated wells at 20,000 cells/well in DMEM/F12 (Thermo Fisher, Gibco, Cat #11320033), 10% FBS (Thermo Fisher, Gibco Cat #10437028), 1% penicillin/streptomycin (Genesee Scientific, GenClone, Cat #25-512). Metabolic activity was measured at 2 and 7 days using the Presto Blue Cell Viability Reagent (Thermo Fisher, Invitrogen, Cat #A13261) according to manufacturer's protocol. Cell number was determined by relating absorbance values back to a known value of cells by creating a standard doubling curve with cells plated from 0 to 160,000 cells/well. Presto Blue dye was removed from the cells, and all wells were washed with 1×PBS. Cells were then placed in cell lysis buffer for subsequent RNA isolation, detailed below, according to RNeasy manufacturer protocol. To visualize the cellular response to the coatings, a live cell tissue imager was utilized (Nikon Eclipse Ti microscope, with an Andor Zyla sCMOS camera, Cat #VSC-02457), an Oko Lab CO2/O2 plate chamber, and air pump (Cat #H201-T-UNIT-BL), and powered by a Peka Light Engine (Lumencor, Cat #3-NII-FA). Over the course of 48 hours, 2,880 images were taken, with the first and last images being the ones displayed in FIG. 13.

Differentiation Assay

Tenocytes and ADSCs were plated on the plates as detailed in above in Cell Proliferation Assay and cultured for 2 or 7 days. RNA was isolated from the plates using RNeasy Mini Kit (Qiagen, Cat #74106) according to manufacturer's protocol. The RNA concentration was measured using the Nano Quant Plate in a Tecan Infinite M200 Pro plate reader. cDNA was reverse transcribed from 250 ng of RNA using qScript cDNA SuperMix (Quanta Bio, Cat #95048) on the ProFlex PCR System (Applied Biosystems, Cat #4483636) according to manufacturer's protocol. Quantitative reverse transcription polymerase chain reaction (qRT-PCR) was performed on a StepOnePlus Real-Time PCR System (Applied Biosystems, Cat #4376592) to measure expression of Scleraxis (Scx: Forward—CGAGAACACCCAAGCCCAAAC, Reverse—CTCCGAATCGCAGTCTTTCTGTC) and Tenomodulin (Tnmd: Forward—TGGGTGGTCCCTCAAGTGAAAGT, Reverse—CTCGACGGCAGTAAATACAACAATA). Scx and Tnmd gene expression was normalized to the housekeeping gene glyceraldehyde 3-phosphate dehydrogenase (GAPDH: Forward—TGACGCTGGGGCTGGCATTG, Reverse—GGCTGGTGGTCCAGGGGTCT). SYBR Green Master Mix (Thermo Fisher, Applied Biosystems, Cat #4368577) was utilized to detect amplicons, and PCR heat cycles were done according to SYBR green manufacturer's protocol. Fold change was calculated using 2^{(-ΔΔCt)}.

Rheometry

Mechanical properties of DTM at different reconstitution levels were measured by rheological testing. DTM was reconstituted at 1 mL, 3 mL, 5 mL and 7 mL per 1 gram of human DTM, or 3 mL per 1 gram of rabbit DTM. Testing was performed on a research rheometer (DHR2, TA Instruments) fitted with a 20 mm scribed plate measuring system, test gap set to 1100 μm. All testing was done at 37° C., and a solvent trap cover was used to minimize drying of the exposed sample. Following a 60 second equilibration time at 37° C., the samples were exposed to oscillatory frequency sweeps from 100 rad/sec to 0.1 rad/sec, logarithmically scaled, 0.1% oscillation strain, 4 points per decade of frequency. Immediately following the oscillation frequency sweep, the samples were exposed to an oscillatory stress sweep ranging from 1.0 Pa to 100,000 Pa, 1 Hz oscillation frequency. A step termination was set such that if at any point the oscillation strain exceeded 1,500%, the test would immediately end. All analyses were performed in duplicate, both immediately after preparation and 30 minutes following preparation. Yield stress values were quantified by fitting an onset model to the complex modulus data. This entailed fitting one straight line through the low stress plateau and a second through the inflection point as the sample yields.

Supraspinatus Tendon Repair in a Rabbit Model

Eight healthy female New Zealand White rabbits (3-3.5 kg, 28-weeks old at time of initial tear) were used for this 12-week study. Rabbits were anaesthetized by subcutaneous injection of Ketamine/Acepromazine, fur was shaved, and the incision site was sterilized with repeated wash. A 2-3 cm skin incision was made, the supraspinatus tendon was detached from the greater tuberosity with a scalpel, and a Penrose drain was inserted into the free end of the tendon to prevent its spontaneous reattachment. The fascia was closed using a 3-0 absorbable braided Vicryl suture, and the wound was closed with buried absorbable 4-0 Vicryl suture in a subcuticular pattern. The rabbits were allowed regular activity for 6 weeks to develop a chronic tendon tear model. After 6 weeks, the rabbits underwent tendon repair surgery. Using the same anesthesia and sterilization techniques as previously stated, the Penrose was identified and removed, and the supraspinatus tendon was fixed to the footprint at the greater tuberosity through 2 transosseous tunnels. The preparation of the greater tuberosity footprint was made by cleaning the remnant soft tendon tissues and trimming the superficial cortical bone in order to have a bleeding subchondral bone. High-strength sutures (#2-0 FiberWire, Arthrex, Naples, Fla., USA) were passed through the transosseous tunnels and supraspinatus tendon and then tied in a standard fashion. After tendon repair surgery, one group received rabbit DTM (1 gram in 3 mL), processed according to the previously described DTM protocol, placed under the supraspinatus tendon near the osteotendinous junction (n=4). The control animals received only the repair procedure (n=4). Animals were euthanized 8-weeks after the repair procedure. The entire humeri and scapulae with their supraspinatus muscle and tendons were harvested for histological analysis. Contralateral shoulders were also collected.

Histological Analysis

Supraspinatus tendons of the repair only group and repair+DTM group were cut to a 1 cm×1 cm squares and cryo-embedded with Neg-50 (Richard-Allan Scientific, Cat #6502). Sections were cut to 6 μm and then stained with Fluroshield Mounting Media with 4′,6-diamidino-2-phenylindole (DAPI; Abcam, Cat #ab104139). Rabbit shoulders were fixed for 2 days in 4% paraformaldehyde (PFA) and decalcified for 4 weeks, shaking at 4° C. in 19% Ethylenediaminetetraacetic acid (EDTA) solution, changing solution every other day. Shoulders were cut down and processed for paraffin embedding using a Tissue-Tek VIP 6 AI Vacuum Infiltration Processor (Sakura, Cat #6040), set to a 1-hour cycle time. Once the bones were embedded in paraffin, sections were cut to 6 μm. Hall Brundt's Quadruple (HBQ) staining of rabbit shoulders was done to visualize cartilage in blue and bone in red. Standard hematoxylin and eosin (H&E) staining was done according to Biospecimen Core Resource Standard Operating Procedure (H001, V4) to visualize matrix staining in pink and nuclei a dark blue/black.

Statistical Analysis

All data was plotted with statistical analysis was performed using Graph Pad Prism 8. Data was plotted so that each donor represents a single dot on a box and whiskers graph in which the central line indicates the median, and the first and third quartiles are the top and bottom of the box, respectively. The whiskers extend to the highest and lowest value observed. Statistical difference was determined by ANOVAs, Tukey's post-hoc comparison, and unpaired t-tests, given the data set and desired definition of relation.

Allograft Sourcing for Decellularized Tendon Matrix (DTM)

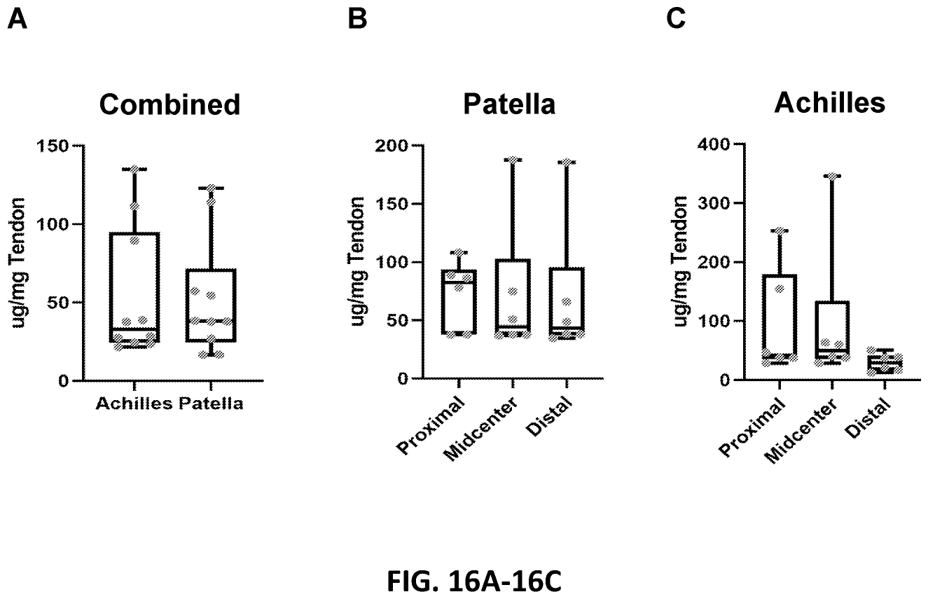

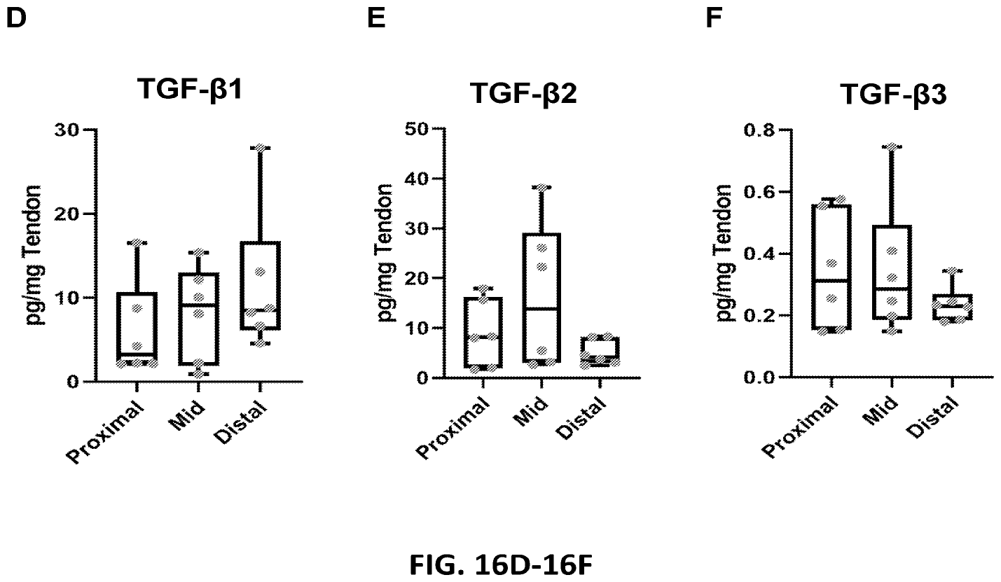

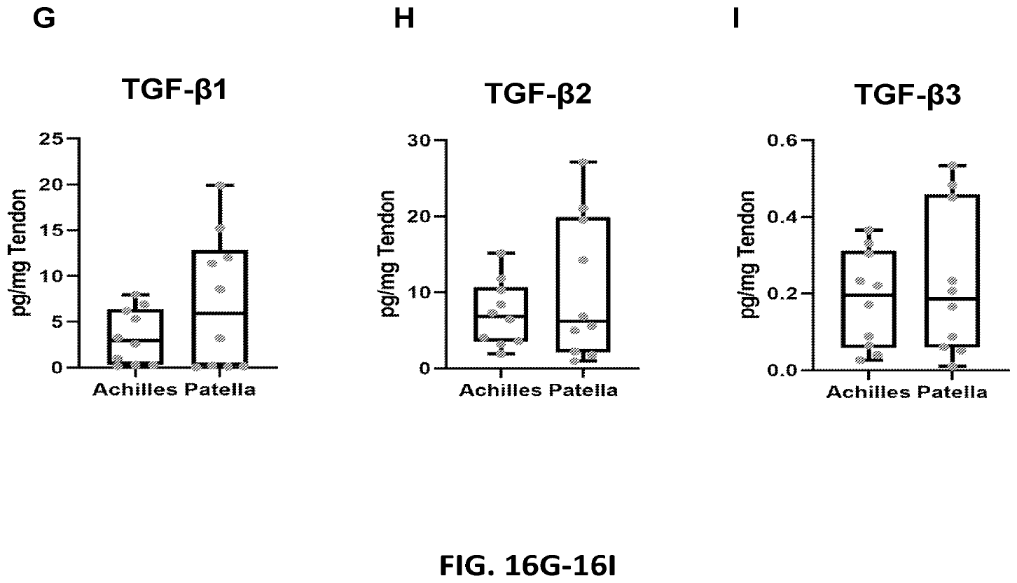

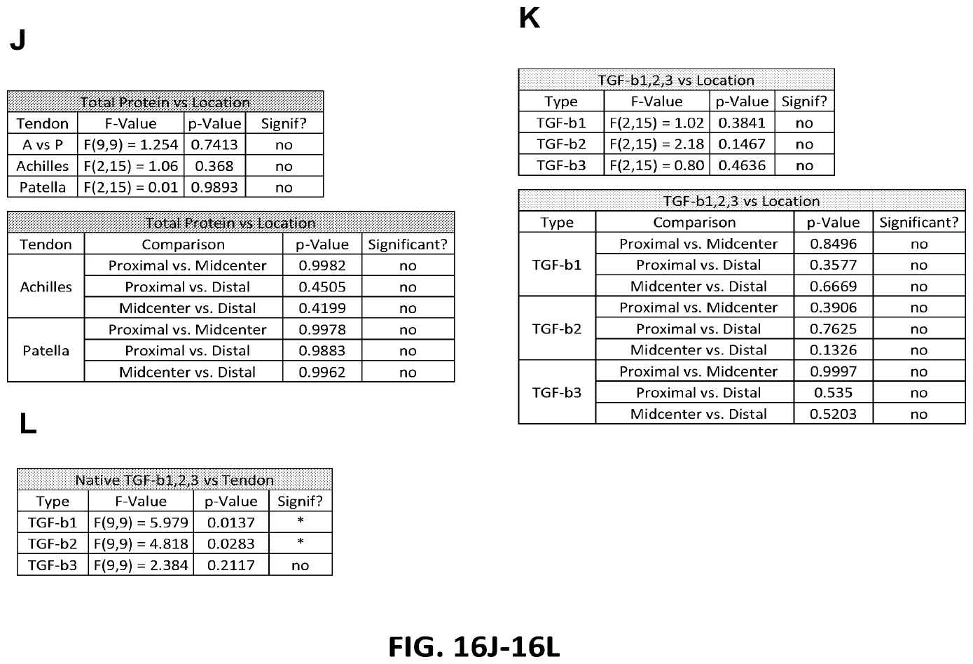

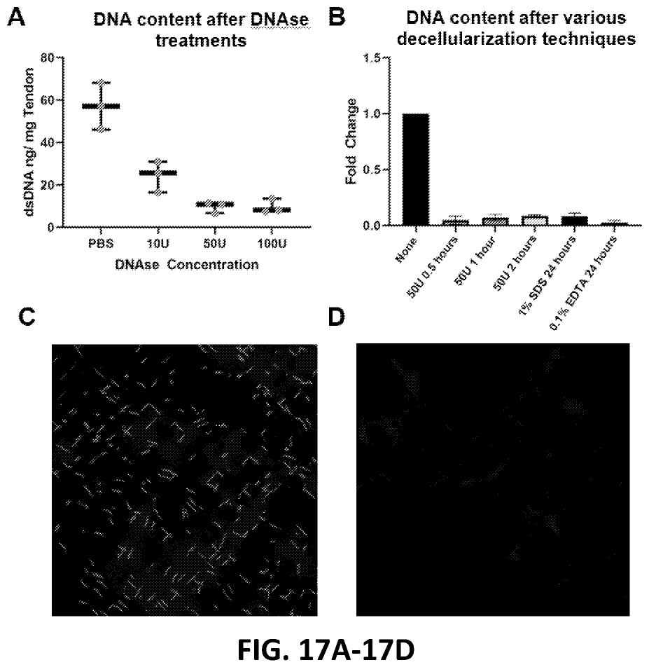

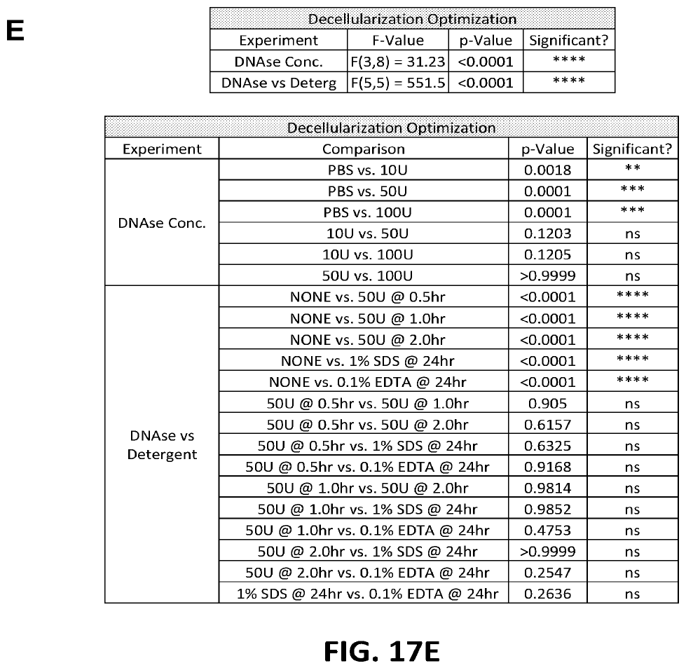

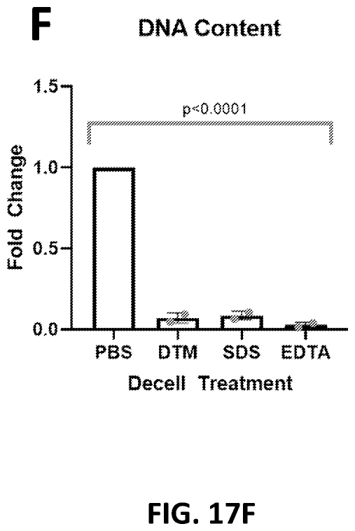

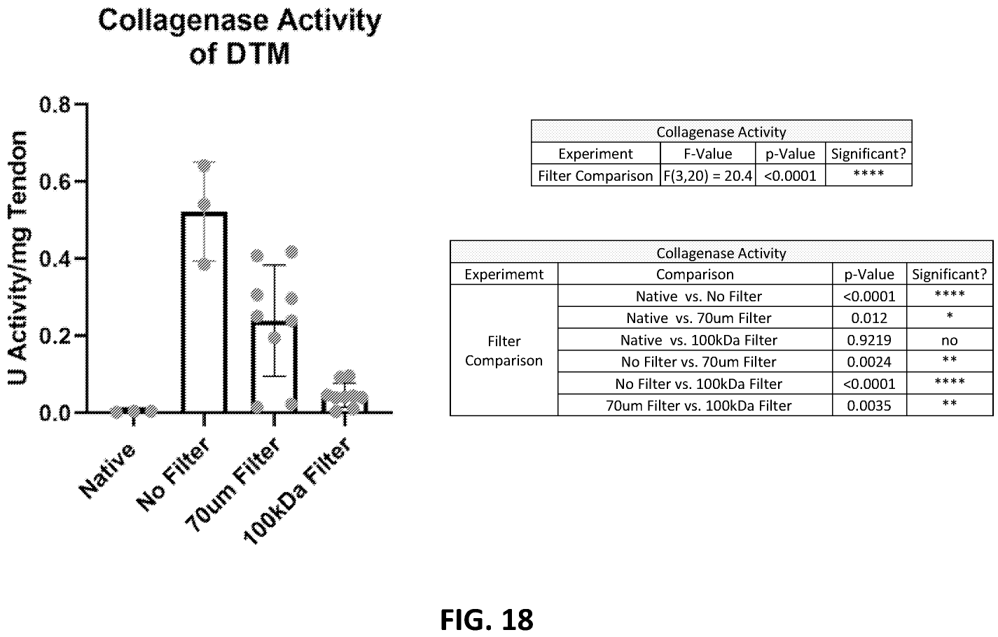

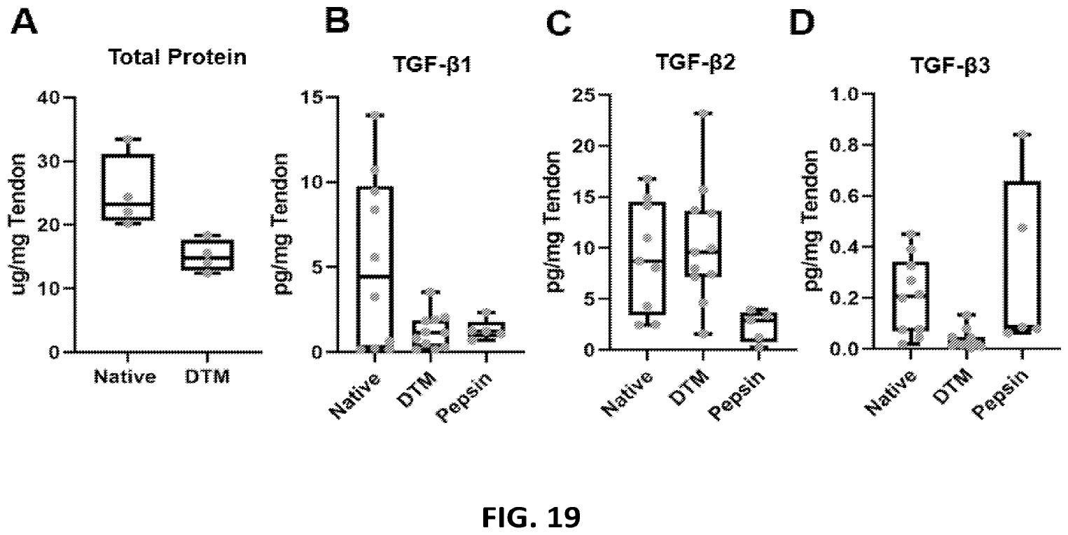

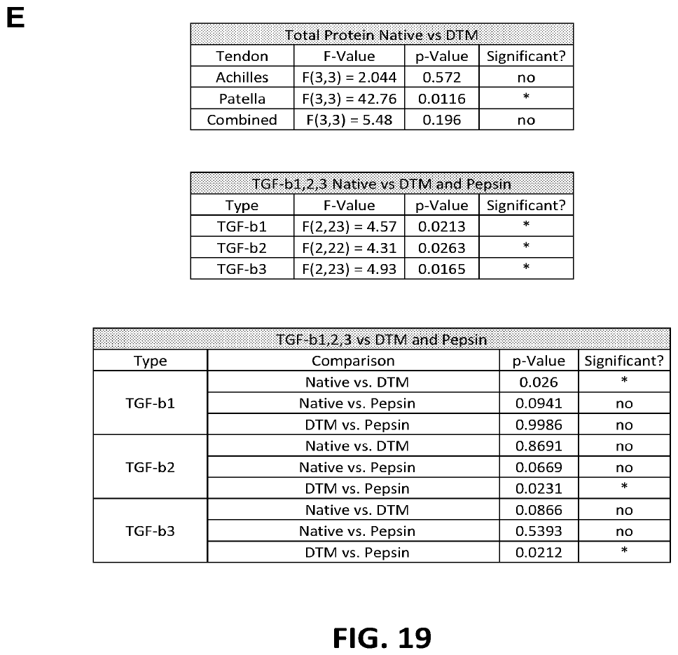

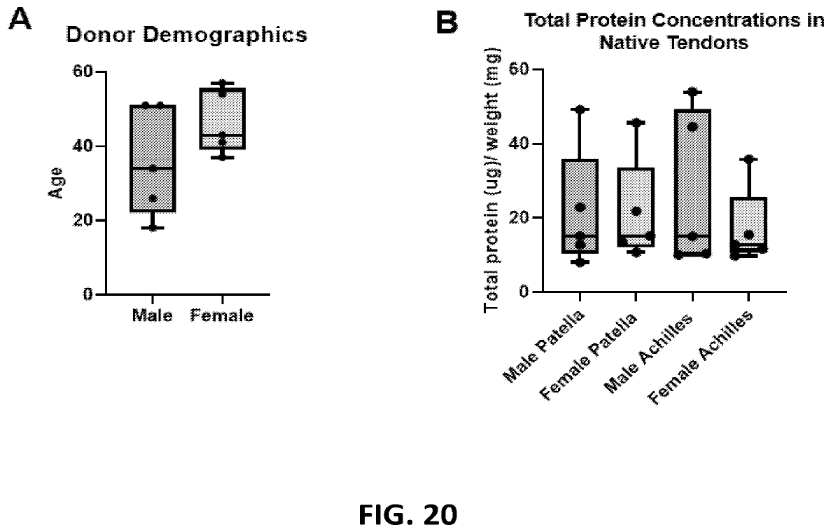

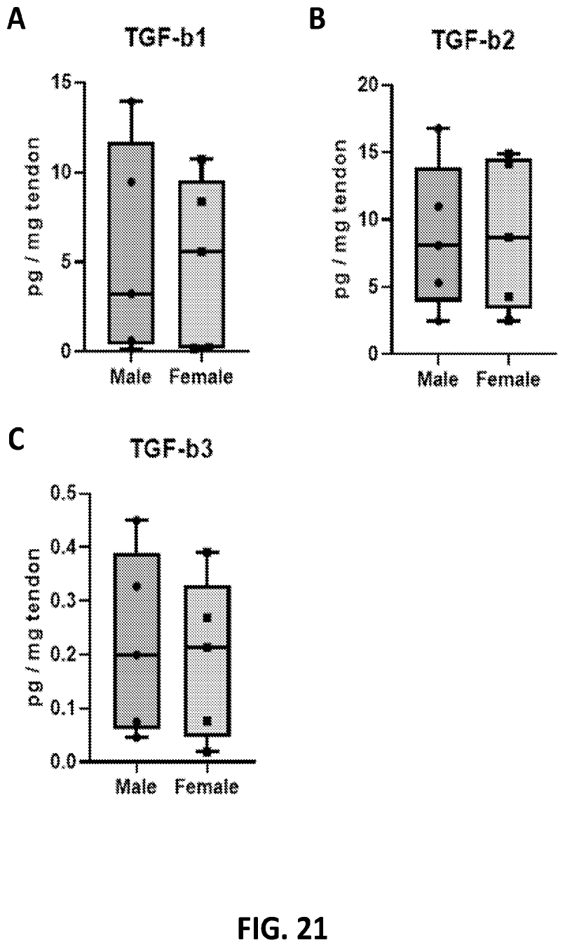

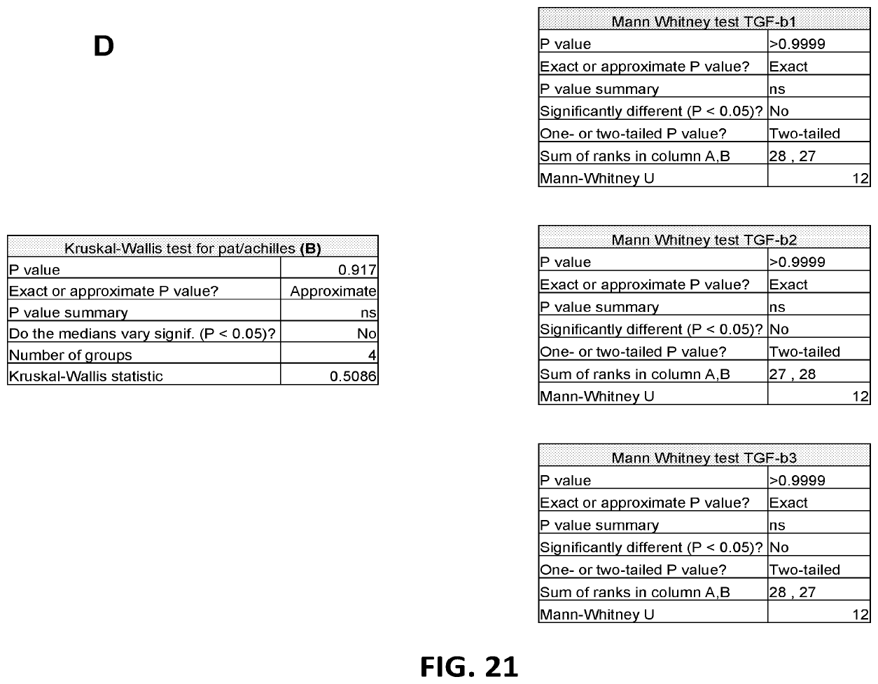

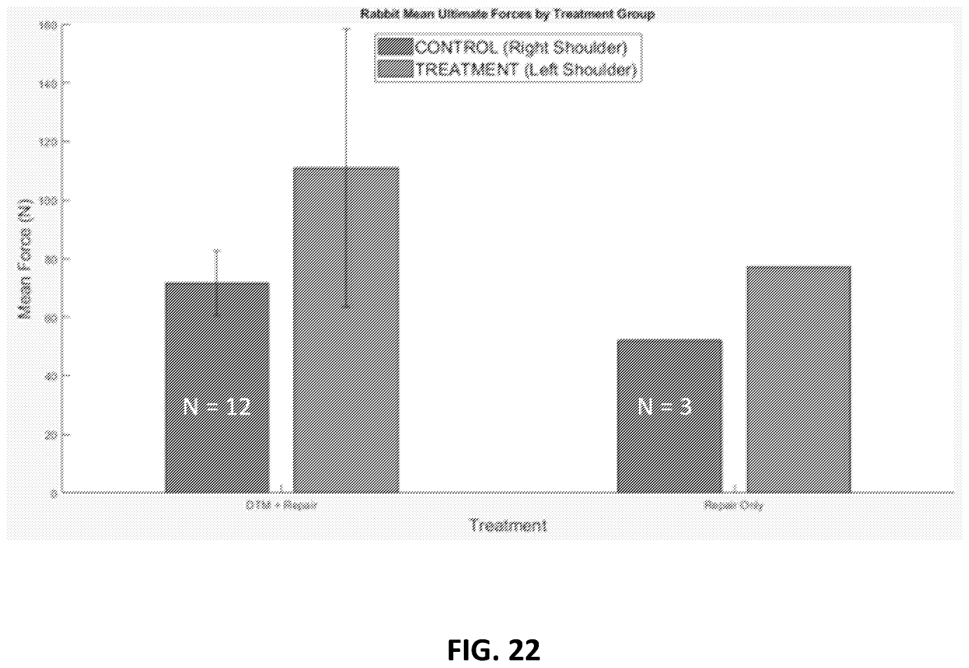

Achilles and patellar tendons are the most readily available, abundant, and accessible allograft tendon tissues. To determine whether there are different levels of native bioactivity across these two tendons, the matrix-bound TGFβ content was measured using Multiplex (n=6-10 individual donor). Further, since it is known that the vascularity of these tendons decreases in the central region, in what is often described as the “watershed zone”, it was aimed to understand if matrix bound TGFβ varied by location within the tendon. Achilles and patellar allografts were grossly dissected into proximal, mid-center, and distal segments based on dividing the total tendon length into thirds (FIG. 23). Tendon within each region was then finely minced and digested into protein isolates for quantification of total protein and TGFβ. No significant difference in total protein content was found between Achilles and patellar tendons (FIG. 16A, p=0.7413) or between proximal, mid-center, and distal divisions within tendons (FIG. 16B-C, p=0.368, and p=0.9893). TGFβ isoforms were then analyzed within protein isolates, and no significant differences were determined in TGFβ content between proximal, mid-center, and distal portions within the Achilles or patellar tendon. However, TGFβ1 and TGFβ2 were significantly higher in the patella tendon compared to the Achilles tendon (FIG. 16G-I, p=0.0137 and p=0.0283, respectively). TGFβ content in the tendons was not differentially expressed according to sex. (FIG. 17, p=0.7413).

Decellularization Step Effectively Removes Cellular Content

To validate the efficacy of the decellularization process, it was compared the specific enzymatic decellularization protocol for DTM to previously published detergent-based techniques that used 1% SDS or 0.1% EDTA for 24 hours. DNA content was quantified using a NanoQuant plate reader following DNA isolation using the DNEasy Blood and Tissue Kit. All decellularization techniques effectively removed DNA relative to PBS (FIG. 17F, p<0.0001), with Tukey's HSD multiple comparison post-hoc testing finding no significant difference between DNA content following decellularization using the DTM process compared to SDS or EDTA (FIG. 17F, p=0.9852 and p=0.4753, respectively). Removal of DNA was confirmed histologically by sectioning and staining native or DTM tendon with DAPI. (FIG. 17C, 17D).

Tendon Processing Maintains TGFβ Bioactivity and Creates a Tendon Putty

To enhance the surgical usability of DTM, it was enzymatically digested decellularized tendon allograft composed of the combined Achilles and patellar tendon to break up the dense matrix and create a moldable form factor. Residual enzymatic activity was removed using a filtration step. (FIG. 10A, p=0.1407) Retention of the key biological growth factor TGFβ in the DTM processed allograft was measured using Multiplex. No significant difference was found between the TGFβ1 (FIG. 10B, p=0.5353) or TGFβ2 (FIG. 10C, p=0.2891) content in the DTM group relative to the native tendon. TGFβ3 showed a significant increase in the DTM compared to the native tendon (FIG. 10D, p=0.0189).

The optimized digestion protocol for DTM generated a malleable putty that can be stretched and reformed, but maintained structural integrity (FIG. 6A, 6B). Rheological properties of the DTM product were measured across various reconstitutions levels (1 gram of tendon diluted in 1, 3, 5, or 7 mL PBS) to determine sample rigidity and structure strength. Oscillation stress was determined by adding an increasing force (1.0-1000,0000 Pa) onto the sample and measuring the elasticity of the sample once the force was removed. The oscillation stress sweeps reveal that samples at all dilutions display elastic dominant behavior across the full range of frequencies applied with overlapping mechanical properties at the 3 and 5 mL dilutions. (FIG. 6F) As expected, the complex modulus plateaus (sample rigidity) and yield stress (sample strength) values decreased with increased dilution. (Table: SUMMARY OF RHEOLOGICAL PROPERTIES OF DTM) Putty-like gross structural properties were maintained in dilutions of 1 gram in 1-5 mL PBS, at 7 mL exceeded the saturation limit of the tissue putty.

SUMMARY OF RHEOLOGICAL PROPERTIES OF DTM

| | | Complex | | |
|---|---|---|---|---|
| | | Modulus Plateau | Phase Angle | Yield Stress |
| | | (Pa) | Plateau (°) | (Pa) |
| A | 1 mL/1 g DTM | 929000 | 11.3 | 12200 |
| B | 3 mL/1 g DTM | 50500 | 15.2 | 683 |
| C | 5 mL/1 g DTM | 32300 | 15.2 | 404 |
| D | 7 mL/1 g DTM | 2640 | 18 | 76 |

DTM Promotes Cell Proliferation and Scleraxis and Tenomodulin Expression

Cellular response to the DTM was characterized in vitro using cell proliferation and gene expression assays. Cellular proliferation was measured using the Presto Blue Assay for both tenocytes, and ADSCs cultured on DTM coated plates and compared back to the standard culture conditions for the individual cell types. On both 2 (FIG. 13A, p=0.0217) and 7 (FIG. 13B, p=0.0004) days following the initial seeding of tenocytes, DTM coated plates had significantly more tenocytes compared to their standard culture conditions (collagenase coated plates). Changes in tenocyte activity and morphology on the individual coatings were visualized using live images taken every 10 minutes over the course of 48 hours. (FIG. 13E-13J) Still, images taken at the time of cell plating and 48 hours later show that tenocytes more rapidly adhere, have increased focal adhesions, and a more native like cell morphology on the DTM (FIG. 13J) compared to the standard tissue culture (FIG. 13H) or collagen coated plate (FIG. 13I). Time lapsed videos show the increased proliferation on the DTM coating that was quantified by Presto Blue (FIG. 13A-B). Similar to tenocytes, ADSCs saw accelerated growth on DTM coated plates relative to control conditions 7 days following plating (FIG. 13D, p=0.0315).

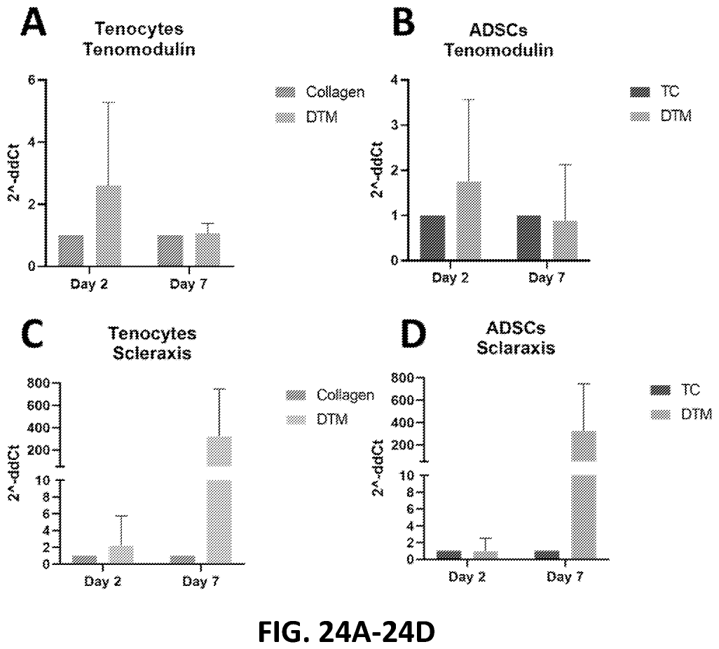

To characterize the cellular phenotype following culture on DTM versus control plates, mRNA was collected from tenocytes and ADSCs following the Presto Blue reaction and expression of the tenogenic markers Tenomodulin (Tnmd) and Scleraxis (Scx) measured using qRT-PCR. Tnmd expression trended higher for both tenocytes and ADSCs cultured on DTM-coated plates, but this trend was not maintained by 7 days in culture. (FIG. 24A-24B) Inversely, Scx expression was unchanged in both cell types on day 2, but by day 7, there is a 100-800-fold higher expression in DTM relative to the control (FIG. 24C-24D).

DTM Enhances Rotator Cuff Repair Improving Regeneration of Enthesis

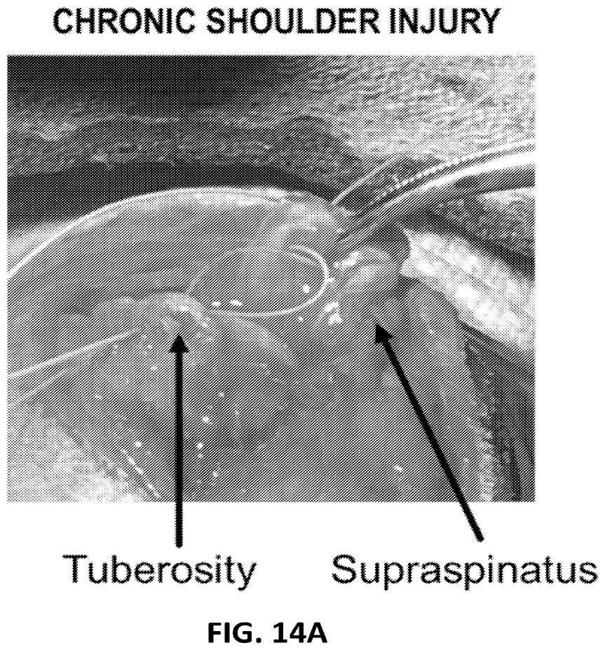

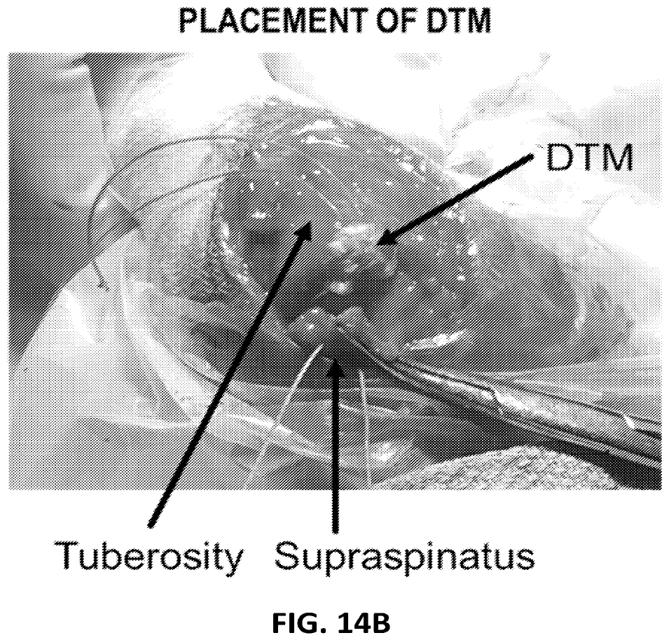

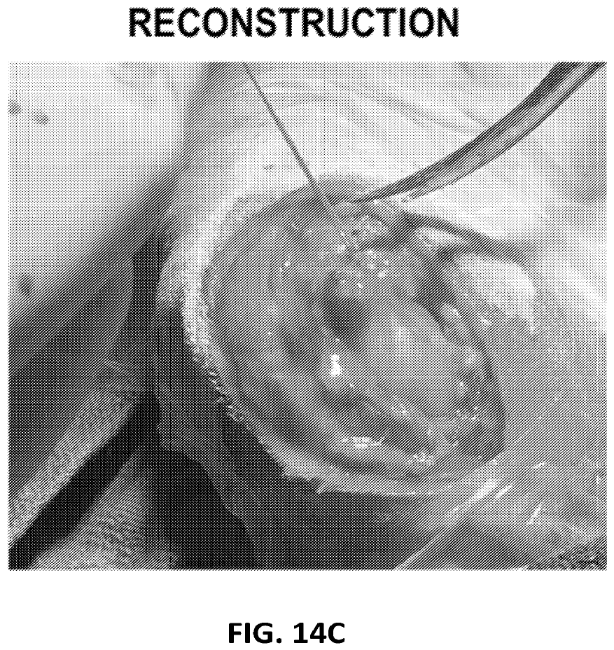

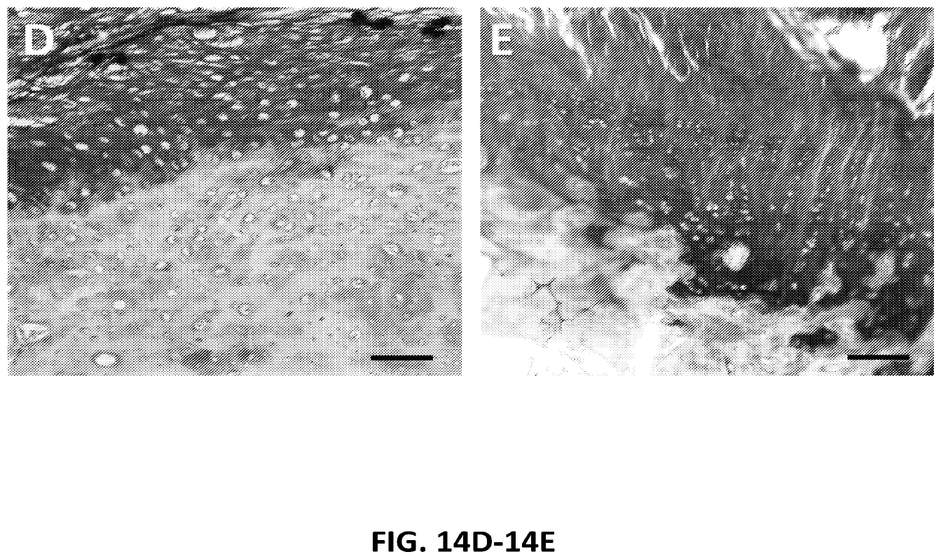

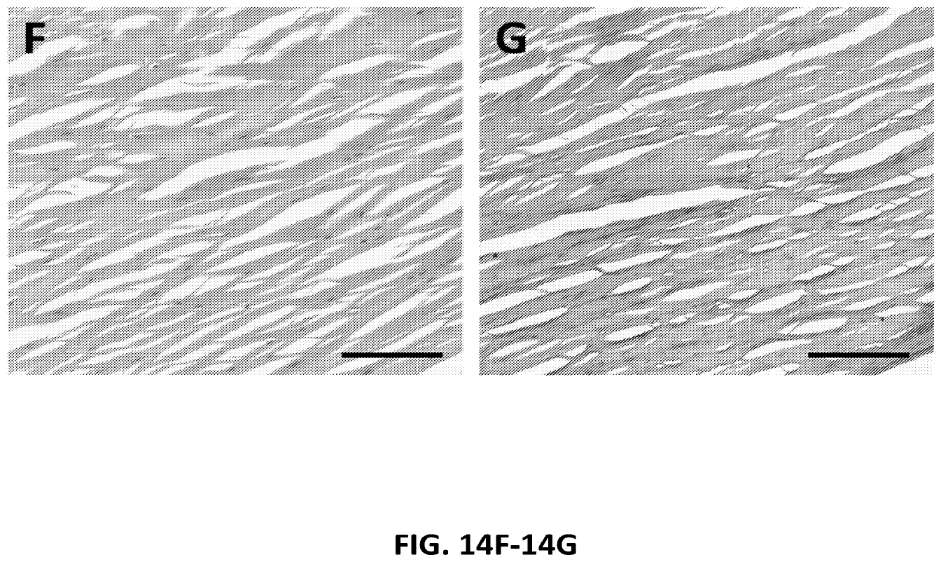

To determine if the in vitro bioactivity translated to a regenerative response, an in vivo pilot study in New Zealand White rabbits was executed. Because many RCR failures are associated with chronic injuries, a rabbit model with a chronic rotator cuff tear was utilized. (FIG. 14A) In the groups that received DTM, the putty (1 mg rabbit DTM diluted in 3 mL PBS) was molded onto the greater tuberosity and the supraspinatus tendon secured to the greater tuberosity as in the control. (FIG. 14B-14C) HBQ staining at the tendon-bone interface shows that DTM promotes formation of a calcified cartilage transition at the junction, indicated by the red staining in the matrix, that is not present in the repair only. (FIG. 9D-E) Within the tendon itself, H&E staining reveals increased cellularity in the tendon augmented with DTM. (FIG. 9F-G).

### Discussion

This study describes the characterization of bioactivity in a novel DTM putty with the long-term goal of promoting tendon regeneration during RCR. Because most rotator cuff tears result in diminished functionality of the shoulder joint and pain, surgical repair remains the gold standard treatment for full thickness rotator cuff tears that appear to be amenable to repair. Unfortunately, RCRs continue to have high clinical failure rates, with the majority of failures occurring at the tendon-bone interface. Despite advancements in surgical technique and instrumentation, retear after RCR remains a concern, particularly in cases of RCR for chronic rotator cuff tear's where the re-rupture rates average over 50% of cases. A fundamental factor driving poor clinical outcomes following rotator cuff repair is that adult tendons have minimal regenerative capacity and heal through fibrous scar tissue formation resulting in inferior biomechanics that leads to partial or full re-tear. Contributing factors include reduced growth factor bioavailability, decreased vascularity in the region of repair, and the absence of the appropriate tendon progenitor cells required for proper tendon regeneration.

Given the importance of tendon healing to success of the RCR, there remains a clinical need to find new, readily translatable approaches to reduces fibrosis and promotes tissue regeneration. Currently, there are few products designed to augment RCR, and those with clinical efficacy studies have reported minimal benefit. The limited use of tendon allografts in clinical practice is likely a result of an early study showing 100% graft failure by MRI evaluation at the 6 months post-operative evaluation following massive RCR with Achilles, patellar, or quadriceps tendon allografts. While recent tendon allograft studies suggest modern surgical techniques can yield functional improvements, MRI images illustrate poor tissue regeneration with tendon allograft and structural failure rate of 57% at 10 years. As an alternative to tendon allografts, dermal and small intestine submucosa allografts have been tested for RCR. While these allografts lack the standalone biomechanical properties of tendons, some early clinical data suggest clinical benefit. However, 3 year follow-up on 24 patients who underwent a mini-open massive RCR using a dermal allograft showed only 24% of patients only had a “partially intact” repair. Large clinical studies on RCR with various types of allograft are lacking, and there are no studies showing strong evidence of tissue regrowth into the allograft or regeneration of the enthesis structure.

In this study, a processing technique to form a decellularized tendon allograft putty (DTM) to promote healing at the tendon-bone interface was developed. Decellularization is considered a critical step in preventing a host inflammatory response to the allograft and disease transmission. Unlike traditional tendon allograft processing techniques, cellular content was removed without detergents in an effort to maintain bioactivity native to the tendon matrix. Previous studies demonstrate detergent-based methods are harsh on the soft tissue matrix of tendons and eliminate proteins and growth factors essential to the healing process. Further, the detergent-based processing time for decellularizing tendon allografts is very long, starting at 24 hours with processing times upwards of one week. The specific, enzymatic decellularization technique was as effective as traditional detergents (SDS and EDTA) at removing cells, and only required 1 hour of treatment time.

Another challenge with traditional tendon allografts is that the form factor is not optimized for augmentation using modern arthroscopic surgical RCR techniques, and cells show little to no ability to penetrate the dense native tendon architecture during repair. Mechanical perforation is one strategy that has been shown to improve penetration of cells into decellularized tendon allografts. Alternatively, enzymatic digestion using pepsin has commonly been used to digest tendon, muscle, and cardiac tissue in order to improve allograft form factor for surgical application and cellular migration. However, pepsin is a non-specific enzyme that indiscriminately cleaves collagenous and non-collagenous matrix proteins altering the bioactivity of the native tendon. Herein, protocols were developed for mechanical and enzymatic processing, filtration, and wash to generate a putty-like tendon allograft with viscoelastic properties similar to the native tendon.

A critical design criterion for DTM was to maintain TGFβ mediated bioactivity found within the native tendon matrix. The focus was on this growth factor because recent mechanistic studies have found TGFβ signaling to be the differential pathway between scarless healing during neonatal tendon regeneration and fibrotic repair in adults. Interestingly, during development, deletion of the TGFβ family leads to a complete loss of all tendons. During neonatal repair, TGFβ promotes tenocyte recruitment, proliferation, and differentiation. TGFβ is temporally regulated to promote healing by stimulating collagen production and angiogenesis. Therapeutically, exogenous TGFβ1 injections were reported to increase collagen type I and III mRNA and to enhance biomechanical function following RCR. To initially determine which tendon would be the best allograft source for the product, the levels of matrix-bound TGFβ in both Achilles and patellar tendon were compared. Differences between the two tendon sources and part of the tendon were not deemed to be functionally substantial; thus, the entire tendon from each source was used to generate the DTM product. Furthermore, all tendon allografts tested were found to contain TGFβ1-3 regardless of the age (18 to 61) or sex of the donor.

Without wishing to be bound by any particular theory, by preserving TGFβ within the DTM product, the bioactivity typically lacking in tendon allografts can be maintained. Specifically, it was demonstrated that cellular proliferation of both tenocytes and ADSCs was improved when cultured on DTM in vitro compared to standard culture conditions. Gene analysis also suggested DTM promoted the expression of Scleraxis (Scx) and Tenomodulin (Tnmd) in both cell types. These tendon-specific transcription factors were tested as they are associated with the development, repair, and maturation of tenocytes. Scx is a transcription factor required for tendon development, and Tnmd represents a set of glycoproteins necessary for tenocyte proliferation and maturation. It has been shown in mice models, that Scx and Tnmd are co-expressed, with scleraxis acting as an early gene marker expressed in tendon progenitors and tenocytes, while tenomodulin acts as a late gene marker indicating tenocyte maturity. This data together suggests, without wishing to be bound by any particular theory, that the processing technique maintains the TGFβ bioactivity in DTM to promote tenocyte and stem cell proliferation, differentiation, and tenocyte maturation.

Preclinical animal studies are a critical step in demonstrating the efficacy of novel technologies for tendon repair. However, unlike many orthopaedic injuries, rodent models have limited applicability for RCR. The rodent supraspinatus has a smaller proportion of coverage and lacks the irreversible fatty infiltration and subsequent poor healing rates observed in humans. As a result, large animal models are typically required to explore efficacy in RCR, and both the rabbit and sheep have been developed as more translationally relevant RCR models. Importantly, chronic injury rabbit models show muscle atrophy, fatty infiltration, and migration of the subscapularis tendon to underneath a bony arch, which are proposed to be highly comparable to massive tears in humans.

For these reasons, a pilot study was performed using the rabbit model of a chronic rotator cuff injury. During repair of the rotator cuff, the DTM putty was spread onto the greater tuberosity, and the supraspinatus tendon was secured to the greater tuberosity using high-strength sutures over 2 transosseous tunnels. This model has previously been shown to allow for pathological changes consistent with chronic rotator cuff tears analogous to a massive rotator cuff injury. This technique is analogous to modern arthroscopic approaches used today to reduce retear rates. The control repair was analogous, but without application of the DTM. Histological repair was assessed following 8 weeks of healing. In the DTM repair group, improved histology of the enthesis was observed. Specifically, the DTM repair animals were found to have longitudinally oriented collagen fibers forming at the enthesis with a zone of calcified cartilage at the bone-to-tendon interface. Gigante et al., reporting on the pathogenesis of rotator cuff tears, consistently found round cells characteristic of a chondrogenic lineage in rotator cuff tears, as compared calcified fibrocartilage typically seen in non-torn tendon enthesis. The results presented similar findings as the RCR only group was shown to have cells phenotypically congruent with chondrocytes lacking the highly aligned calcified fibrocartilage region of a normal enthesis. Failure of RCR in chronic/massive tears has specifically been associated with the formation of mechanically weak fibrovascular scars lacking the zone of calcified cartilage. Furthermore, more cells in the tendon region near the DTM repair compared to the RCR alone were observed. This observation is consistent with in vitro data suggesting DTM promotes cellular proliferation.

DTM represents an adaptation to allograft processing that can maintain bioactivity to promote better tissue regeneration. By utilizing tendon allografts as the basis for the DTM, it was aimed to maintain the tissue-specific bioactivity of the tendon compared to the ectopic tissues (e.g., dermal or intestinal submucosa) basis used in clinical practice.

### Example 6 Measuring Mechanical Properties of Decellularized Tendon Matrix (DTM)—Rheological Investigation

To describe the optimal reconstitution concentration of Decellularized Tendon Matrix (DTM) putty, biomechanical properties of various concentrations of DTM in the proposed solvent, PBS, are quantified using rheology. Rheological tests can be designed and results interpreted according to, for example, the following: azom.com/article.aspx?ArticleID=10219, which is incorporated by reference herein in its entirety for all purposes.

Samples that are tested include, 1) samples that behave more of a liquid, 2) samples that have more liquid than optimal concentration, 3) samples that are at optimal concentration, 4) samples that are more solid than optimal concentration, and 5) samples that behave more of a solid.

Various rheological tests are performed. For example, 1) viscosity across a range of shear conditions, wherein zero shear viscosity refers to the viscosity of the material at rest and is a major determinant of resistance to creep, and wherein when the viscosity is higher at lower shear stresses, there is higher resistance for deformations; 2) yield stress tests which measures the strength of a gel or matrix and indicates the stress required to initiate flow, wherein a higher value of yield stress suggests a more stable sample; and 3) optimization of viscoelastic properties, wherein a viscoelastic spectrum of the material is determined through performing a frequency sweep, wherein a solid-like behaviour at low frequencies indicates stability. A low phase angle suggests the material behaves as a solid, not a liquid.

Several concentrations of DTM are tested and each sample's solid vs. liquid behavior, strength and stability is characterized through performing rheology. Generally speaking, rheological testing measures the amount of deformation following an applied stress to the material.

This report describes the testing performed on the five supplied samples of soft tissue product with the aim of identifying the rheological correlates to handling properties. The main rheological properties investigated were sample rigidity and structure strength under low stress conditions. The supplied samples studied were labelled:
A—Concentration 1 ml/1 g HUMAN
B—Concentration 3 ml/1 g HUMAN
C—Concentration 5 ml/1 g HUMAN
D—Concentration 7 ml/1 g HUMAN
E—Concentration 3 ml/1 g RABBIT

Equipment and Methods

Testing was performed on a research rheometer (DHR2, TA Instruments) fitted with a 20 mm scribed plate measuring system, test gap set to 1100 μm. Testing was performed at 37° C. A solvent trap cover was employed to minimize drying of the sample at the exposed edge.

The samples were re-constituted by combining the contents of the matching vials and mixing vigorously with a vortex mixer for 60 s before loading an aliquot onto the rheometer for analysis.

Oscillation Frequency Sweeps

Following a 60 s equilibration time at 37° C. the samples were exposed to oscillatory frequency sweeps, 100 rad/s to 0.1 rad/s, logarithmically scaled, 0.1% oscillation strain, 4 points per decade of frequency.

Oscillation Stress Sweeps

Immediately following on from the oscillation frequency sweep the samples were exposed to an oscillatory stress sweep ranging from 1.0 Pa to 100,000 Pa, 1 Hz oscillation frequency. A step termination was set such that if at any point the oscillation strain exceeded 1500% the test would immediately end.

These analyses were performed in duplicate on the samples immediately after preparation, and then repeated 30 minutes after preparation.

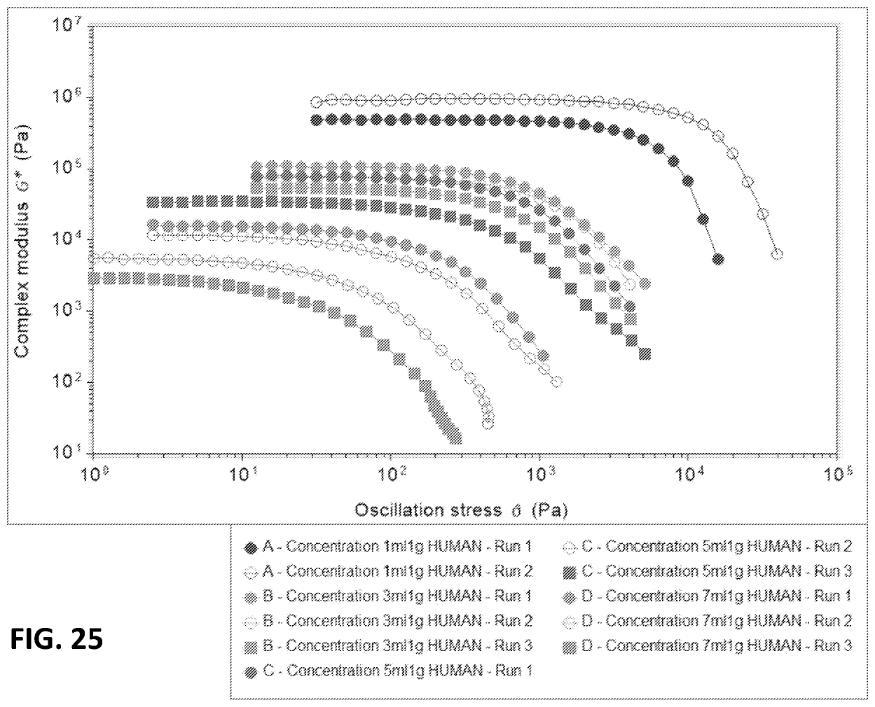

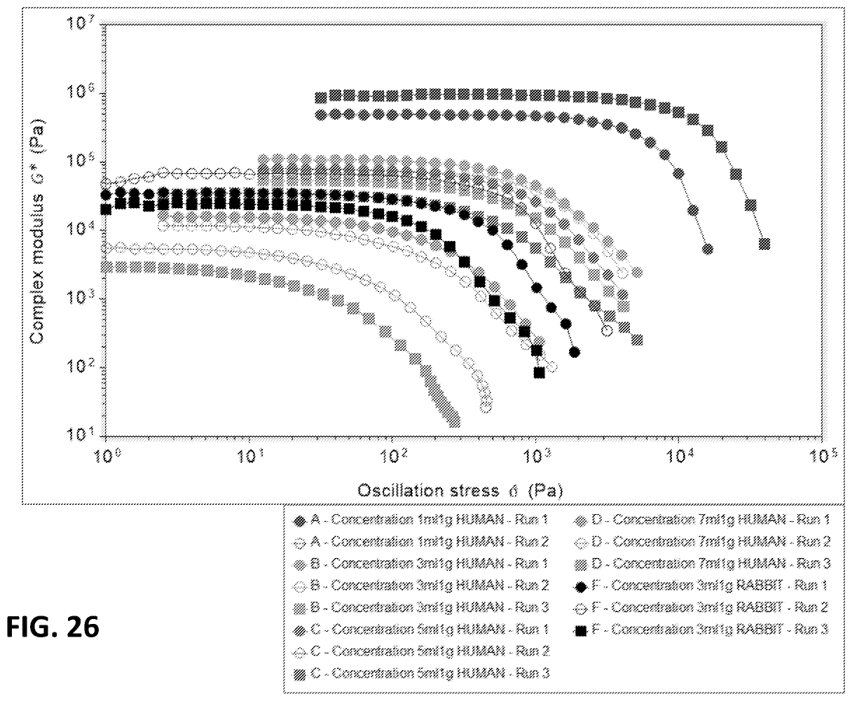

Oscillation Stress Sweeps: FIG. 25 illustrates the Complex Modulus (Pa) v Oscillation Stress (Pa); FIG. 26 illustrates Complex Modulus (Pa) v Oscillation Stress (Pa)—All Samples;

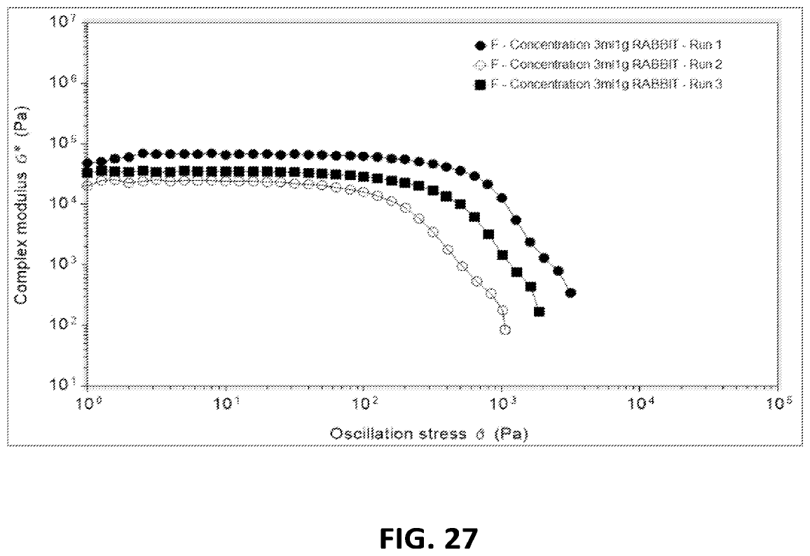

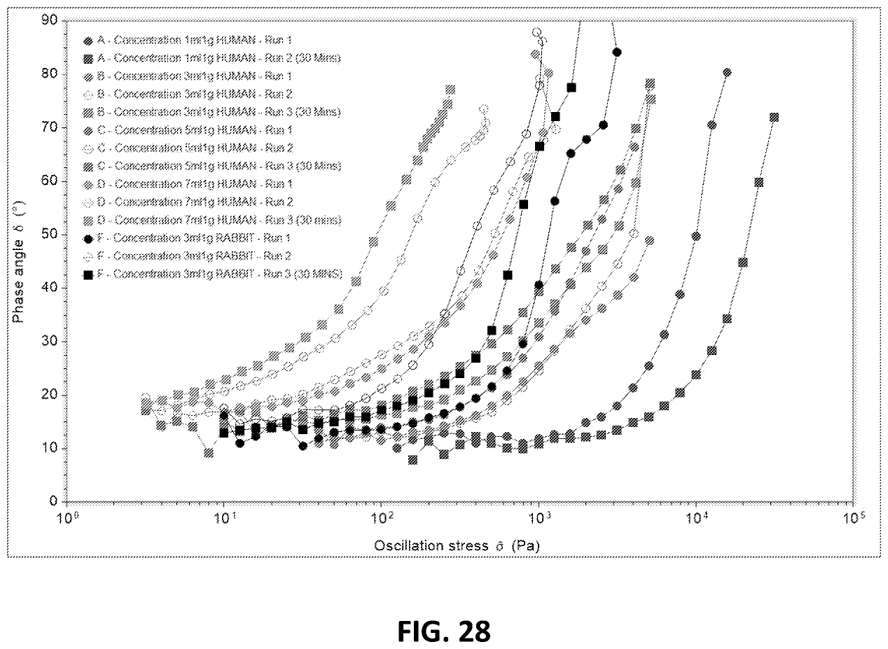

FIG. 27 illustrates Complex Modulus (Pa) v Oscillation Stress (Pa)—Rabbit sample only; FIG. 28 illustrates Phase Angle (°) v Oscillation Stress (Pa)

Plateau values for freshly prepared material

| | Complex Modulus Plateau (Pa) | | | Phase Angle Plateau (°) | | |
|---|---|---|---|---|---|---|
| Sample | Run 1 | Run 2 | Mean | Run 1 | Run 2 | Mean |
| A - Concentration 1 ml/1 g HUMAM | 479000 | — | 479000 | 12.2 | — | 12.2 |
| B - Concentration 3 ml/1 g HUMAN | 104000 | 75000 | 89400 | 12.5 | 12.8 | 12.7 |
| C - Concentration 5 ml/1 g HUMAN | 74200 | 10500 | 42400 | 14.1 | 16.2 | 15.2 |
| D - Concentration 7 ml/1 g HUMAN | 14600 | 5050 | 9810 | 20.0 | 17.6 | 18.8 |
| E - Concentration 3 ml/1 g RABBIT | 66100 | 22700 | 44400 | 13.2 | 16.6 | 14.9 |

Plateau values of aged material

| | Complex Modulus | Phase Angle |
|---|---|---|
| | Plateau (Pa) | Plateau (°) |
| | (After 30 | (After 30 |
| Sample | mins) | mins) |
| A - Concentration 1 ml/1 g HUMAN | 929000 | 11.3 |
| B - Concentration 3 ml/1 g HUMAN | 50500 | 15.2 |
| C - Concentration 5 ml/1 g HUMAN | 32300 | 15.2 |
| D - Concentration 7 ml/1 g HUMAN | 2640 | 18.0 |
| E - Concentration 3 ml/1 g RABBIT | 33500 | 14.7 |

Yield stress of freshly prepared material

| | Yield Stress (Pa)* | | |
|---|---|---|---|
| Sample | Run 1 | Run 2 | Mean |
| A - Concentration 1 ml/1 g HUMAN | 6840 | — | 6840 |
| B - Concentration 3 ml/1 g HUMAN | 797 | 1090 | 941 |
| C - Concentration 5 ml/1 g HUMAN | 788 | 145 | 467 |
| D - Concentration 7 ml/1 g HUMAN | 270 | 212 | 241 |
| E - Concentration 3 ml/1 g RABBIT | 668 | 154 | 411 |

Yield stress of aged material

| | Yield Stress (Pa)* |
|---|---|
| Sample | (After 30 mins) |
| A - Concentration 1 ml/1 g HUMAN | 12200 |
| B - Concentration 3 ml/1 g HUMAN | 683 |
| C - Concentration 5 ml/1 g HUMAN | 404 |
| D - Concentration 7 ml/1 g HUMAN | 76.0 |
| E - Concentration 3 ml/1 g RABBIT | 381 |

*Yield stress values were quantified by fitting an onset model to the complex modulus data. This entailed fitting one straight line through the low stress plateau and a second through the inflection point as the sample yields.

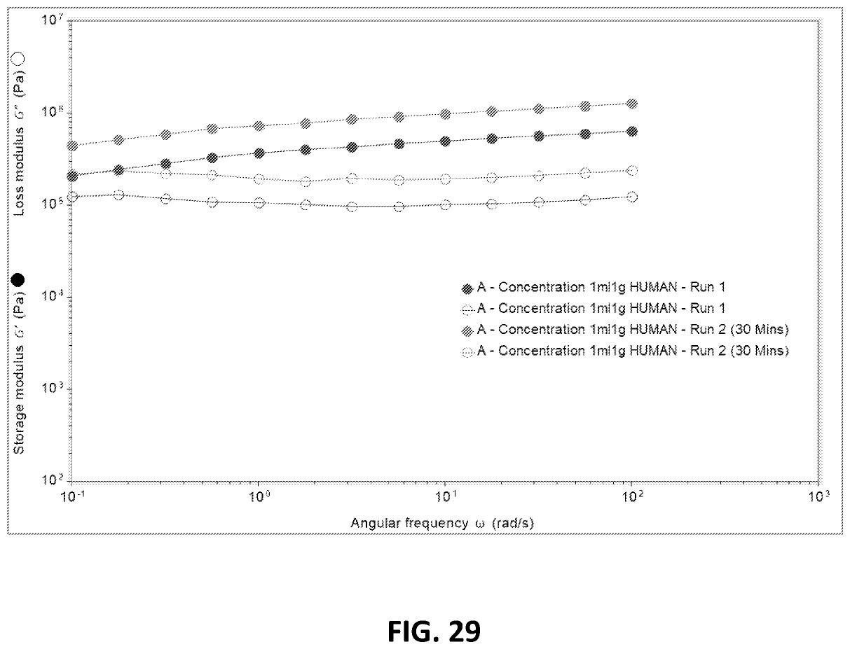

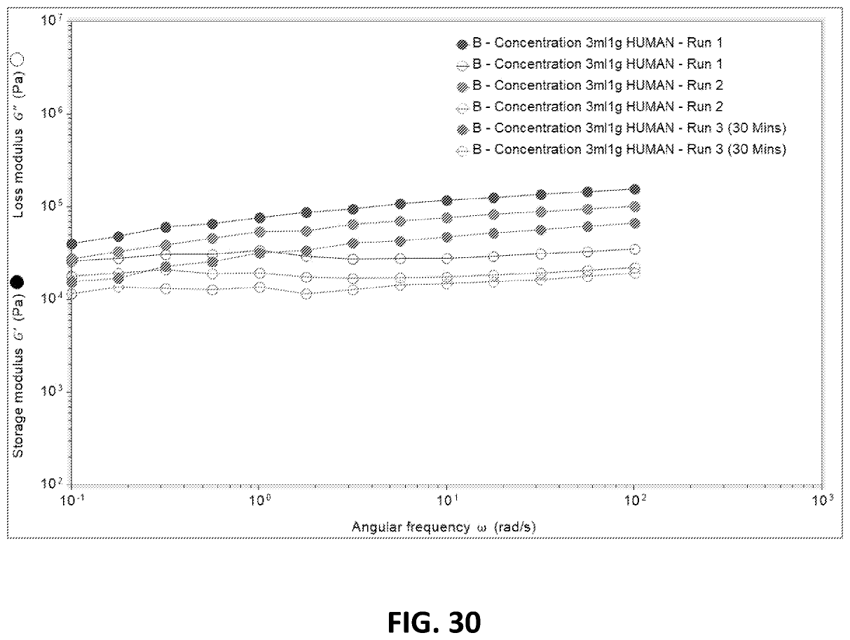

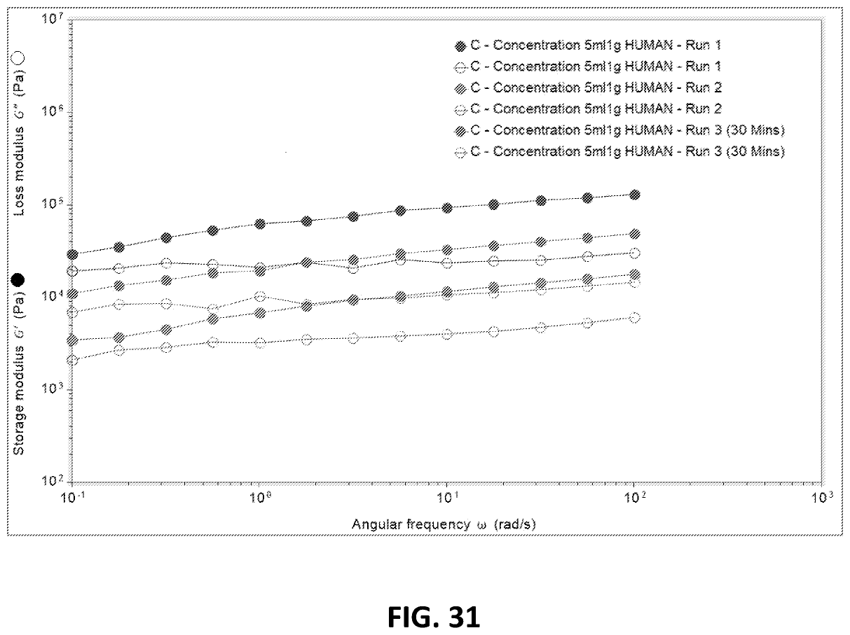

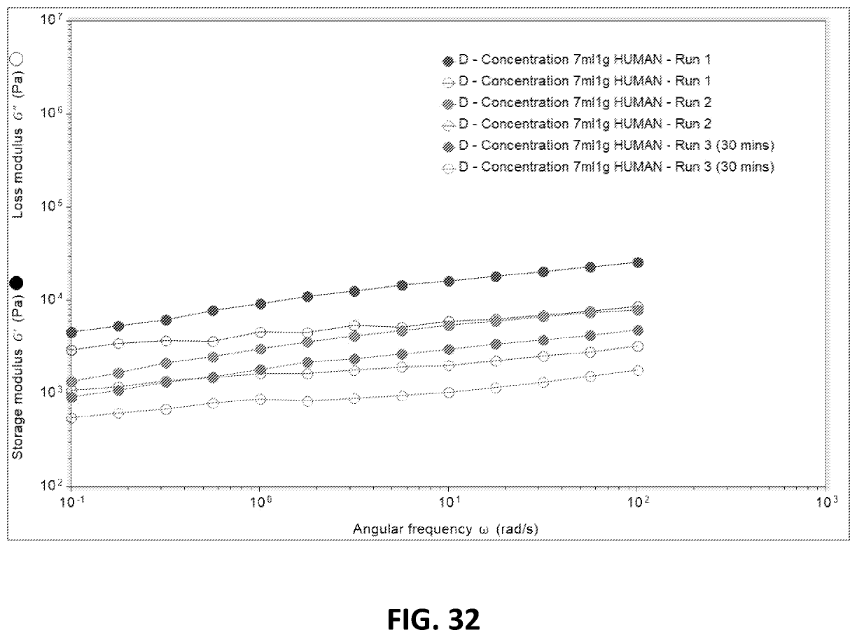

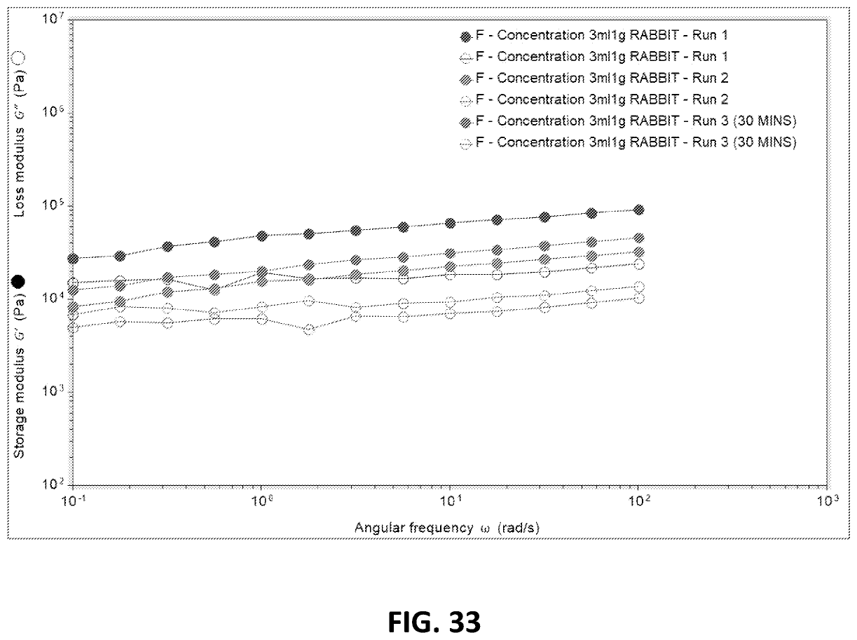

Oscillation Frequency Sweeps: FIG. 29 illustrates Storage Modulus (Pa) and Loss Modulus (Pa) v Angular Frequency (rad/s)—Sample A; FIG. 30 illustrates Storage Modulus (Pa) and Loss Modulus (Pa) v Angular Frequency (rad/s)—Sample B; FIG. 31 illustrates Storage Modulus (Pa) and Loss Modulus (Pa) v Angular Frequency (rad/s)—Sample C; FIG. 32 illustrates Storage Modulus (Pa) and Loss Modulus (Pa) v Angular Frequency (rad/s)—Sample D; FIG. 33 illustrates Storage Modulus (Pa) and Loss Modulus (Pa) v Angular Frequency (rad/s)—Sample E.

In some embodiments, the oscillation stress sweeps reveal that the samples display well formed elastic structure under low stress conditions. In some embodiments, the complex modulus plateaus (sample rigidity) and yield stress (sample strength) values decreased with increased dilution. The results from testing Sample E—Concentration 3 ml/1 g RABBIT, indicate it is most like Sample C—Concentration 5 ml/1 g HUMAN in its structural properties. The results of the oscillation frequency sweeps show a range of modulus values for the samples that correlate with the results seen for the oscillation stress sweeps. The samples display elastic dominant behavior across the full range of frequencies applied.

Oscillation Stress Sweep

The oscillation stress sweep test provides a simple quantification of the rigidity and strength of soft solid structure present throughout a sample. The test entails the application of small, incrementing sinusoidal (i.e., clockwise then counter clockwise) shear stresses to the sample whilst monitoring its resulting deformation and/or flow. In the early stages of the test the stress is sufficiently low to preserve structure. The presence of this structure is revealed by dominant elastic deformation (rather than viscous flow) signified by a phase angle plateau at low values. Phase angle is a measure of the relative dominance of elastic or viscous response of the sample and ranges from 0° for an ideal elastic material (i.e. a perfect solid) to 90° for an ideal viscous material (a perfect liquid). At this stage the sample rigidity, the complex modulus, also remains at a plateau value. As the test progresses the incrementing applied stress eventually disrupts sample structure as the yielding process progresses.

This is manifested as a loss of elastic response (phase angle rises) and an accompanying decrease in rigidity (complex modulus decreases).

The oscillation stress sweep may also be presented as storage modulus (G′) and loss modulus (G″) as a function of applied stress. Storage and loss modulus are measures of the respective abilities for the material to store energy through elastic deformation or dissipate energy through viscous flow during each oscillatory deformation cycle.

Oscillation Frequency Sweep

The oscillatory frequency sweep entails applying small, sinusoidal (clockwise then counter-clockwise) strains to a sample, sweeping the frequency of oscillation and monitoring the resulting stress response, from which viscoelastic information can be gained. The test is used to identify the relative proportions of viscous or elastic behavior across a range of deformation timescales.

Results from the oscillatory frequency sweeps are usually presented as viscoelasticity v timescale profiles of storage (G′) and loss modulus (G″) v frequency. Storage and loss modulus are measures of the respective abilities for the material to store energy through elastic deformation or dissipate energy through viscous flow during each oscillatory deformation cycle. In simple terms the test can establish the “dominant viscoelastic response” of a material.

### REFERENCES

1 Yelin, E., Weinstein, S. & King, T. The burden of musculoskeletal diseases in the United States. Semin Arthritis Rheum 46, 259-260, doi:10.1016/j.semarthrit.2016.07.013 (2016).
2 Benjamin, M., Kaiser, E. & Milz, S. Structure-function relationships in tendons: a review. J Anat 212, 211-228, doi:10.1111/j.1469-7580.2008.00864.x (2008).
3 Jósza, L. et al. A comparative study of methods for demonstration and quantification of capillaries in skeletal muscle. Acta Histochemica 94, 89-96, doi:10.1016/s0065-1281(11)80344-2 (1993).
4 Inc., i. R. U.S. MARKET REPORT SUITE FOR ORTHOPEDIC SOFT TISSUE REPAIR. 30-36 (2019).
5 Rashid, M. S. et al. Increasing age and tear size reduce rotator cuff repair healing rate at 1 year. Acta Orthop 88, 606-611, doi:10.1080/17453674.2017.1370844 (2017).
6 Aguado, G., Obando, D. V., Herrera, G. A., Ramirez, A. & Llinás, P. J. Retears of the Rotator Cuff: An Ultrasonographic Assessment During the First Postoperative Year. Orthop J Sports Med 7, 2325967119889049, doi:10.1177/2325967119889049 (2019).
7 Katthagen, J. C., Bucci, G., Moatshe, G., Tahal, D. S. & Millett, P. J. Improved outcomes with arthroscopic repair of partial-thickness rotator cuff tears: a systematic review. Knee Surg Sports Traumatol Arthrosc 26, 113-124, doi:10.1007/s00167-017-4564-0 (2018).
8 Galatz, L. M., Ball, C. M., Teefey, S. A., Middleton, W. D. & Yamaguchi, K. The outcome and repair integrity of completely arthroscopically repaired large and massive rotator cuff tears. J Bone Joint Surg Am 86, 219-224, doi:10.2106/00004623-200402000-00002 (2004).
9 Millett, P. J., Horan, M. P., Maland, K. E. & Hawkins, R. J. Long-term survivorship and outcomes after surgical repair of full-thickness rotator cuff tears. J Shoulder Elbow Surg 20, 591-597, doi:10.1016/j.jse.2010.11.019 (2011).
10 Zumstein, M. A., Jost, B., Hempel, J., Hodler, J. & Gerber, C. The clinical and structural long-term results of open repair of massive tears of the rotator cuff. J Bone Joint Surg Am 90, 2423-2431, doi:10.2106/jbjs.G.00677 (2008).
11 Collin, P. et al. Clinical and structural outcome 20 years after repair of massive rotator cuff tears. J Shoulder Elbow Surg 29, 521-526, doi:10.1016/j.jse.2019.07.031 (2020).
12 Lafosse, L., Brozska, R., Toussaint, B. & Gobezie, R. The outcome and structural integrity of arthroscopic rotator cuff repair with use of the double-row suture anchor technique. J Bone Joint Surg Am 89, 1533-1541, doi:10.2106/jbjs.F.00305 (2007).
13 McColl, A. H., Lam, P. H. & Murrell, G. A. C. Are we getting any better? A study on repair integrity in 1600 consecutive arthroscopic rotator cuff repairs. JSES Open Access 3, 12-20, doi:10.1016/j.jses.2019.01.002 (2019).
14 Kunze, K. N., Rossi, L. A., Beletsky, A. & Chahla, J. Does the Use of Knotted Versus Knotless Transosseous Equivalent Rotator Cuff Repair Technique Influence the Incidence of Retears? A Systematic Review. Arthroscopy 36, 1738-1746, doi:10.1016/j.arthro.2020.01.052
15 Benjamin, M. et al. Where tendons and ligaments meet bone: attachment sites (‘entheses’) in relation to exercise and/or mechanical load. Journal of Anatomy 208, 471-490, doi:https://doi.org/10.1111/j.1469-7580.2006.00540.x (2006).
16 Bunker, T. in European Surgical Orthopaedics and Traumatology: The EFORT Textbook (ed George Bentley) 1043-1061 (Springer Berlin Heidelberg, 2014).
17 Gerber, C., Schneeberger, A. G., Perren, S. M. & Nyffeler, R. W. Experimental rotator cuff repair. A preliminary study. The Journal of bone and joint surgery. American volume 81, 1281-1290, doi:10.2106/00004623-199909000-00009 (1999).
18 Kovacevic, D. & Rodeo, S. A. Biological augmentation of rotator cuff tendon repair. Clinical orthopaedics and related research 466, 622-633, doi:10.1007/s11999-007-0112-4 (2008).
19 Howell, K. et al. Novel Model of Tendon Regeneration Reveals Distinct Cell Mechanisms Underlying Regenerative and Fibrotic Tendon Healing. Sci Rep 7, 45238, doi:10.1038/srep45238 (2017).
20 Galatz, L. M., Gerstenfeld, L., Heber-Katz, E. & Rodeo, S. A. Tendon regeneration and scar formation: The concept of scarless healing. Journal of Orthopaedic Research 33, 823-831, doi:https://doi.org/10.1002/jor.22853 (2015).
21 Huang, A. H., Lu, H. H. & Schweitzer, R. Molecular regulation of tendon cell fate during development. Journal of Orthopaedic Research 33, 800-812, doi:https://doi.org/10.1002/jor.22834 (2015).
22 Huang, A. H. et al. Requirement for scleraxis in the recruitment of mesenchymal progenitors during embryonic tendon elongation. Development 146, doi:10.1242/dev.182782 (2019).
23 Deepak A Kaji, K. L. H., Zerina Balic, Dirk Hubmacher, Alice H Huang. Tgfβ signaling is required for tenocyte recruitment and functional neonatal tendon regeneration. eLife 2020, doi:10.7554/eLife.51779 (2020).
24 Thangarajah, T. & Lambert, S. Management of the unstable shoulder. BMJ: British Medical Journal 350, h2537, doi:10.1136/bmj.h2537 (2015).
25 Moore, M. S., McAuley, J. P., Young, A. M. & Engh, C. A., Sr. Radiographic signs of osseointegration in porous-coated acetabular components. Clinical orthopaedics and related research 444, 176-183, doi:10.1097/01.blo.0000201149.14078.50 (2006).
26 Farnebo, S., Woon, C. Y., Kim, M., Pham, H. & Chang, J. Reconstruction of the Tendon&#x2013; Bone Insertion With Decellularized Tendon&#x2013; Bone Composite Grafts: Comparison With Conventional Repair. Journal of Hand Surgery 39, 65-74, doi:10.1016/j.jhsa.2013.09.033 (2014).
27 Spang, C., Chen, J. & Backman, L. J. The tenocyte phenotype of human primary tendon cells in vitro is reduced by glucocorticoids. BMC Musculoskelet Disord 17, 467, doi:10.1186/s12891-016-1328-9 (2016).
28 Hall, B. K. The role of movement and tissue interactions in the development and growth of bone and secondary cartilage in the clavicle of the embryonic chick. Journal of Embryology and Experimental Morphology 93, 133-152 (1986).
29 Lovati, A. B., Bottagisio, M. & Moretti, M. Decellularized and Engineered Tendons as Biological Substitutes: A Critical Review. Stem Cells Int 2016, 7276150, doi:10.1155/2016/7276150 (2016).
30 Shin, Y. K. et al. Predictive Factors of Retear in Patients With Repaired Rotator Cuff Tear on Shoulder Mitt American Journal of Roentgenology 210, 134-141, doi:10.2214/AJR.17.17915 (2017).
31 Kaji, D. A., Howell, K. L., Balic, Z., Hubmacher, D. & Huang, A. H. Tgfβ signaling is required for tenocyte recruitment and functional neonatal tendon regeneration. Elife 9, doi:10.7554/eLife.51779 (2020).
32 Docheva, D., Müller, S. A., Majewski, M. & Evans, C. H. Biologics for tendon repair. Adv Drug Deliv Rev 84, 222-239, doi:10.1016/j.addr.2014.11.015 (2015).
33 Cho, N. S., Yi, J. W. & Rhee, Y. G. Arthroscopic Biceps Augmentation for Avoiding Undue Tension in Repair of Massive Rotator Cuff Tears. Arthroscopy: The Journal of Arthroscopic & Related Surgery 25, 183-191, doi:https://doi.org/10.1016/j.arthro.2008.09.012 (2009).
34 Ricchetti, E. T., Aurora, A., Iannotti, J. P. & Derwin, K. A. Scaffold devices for rotator cuff repair. J Shoulder Elbow Surg 21, 251-265, doi:10.1016/j.jse.2011.10.003 (2012).
35 Aurora, A., McCarron, J., Iannotti, J. P. & Derwin, K. Commercially available extracellular matrix materials for rotator cuff repairs: state of the art and future trends. J Shoulder Elbow Surg 16, S171-178, doi:10.1016/j.jse.2007.03.008 (2007).
36 Longo, U. G., Lamberti, A., Maffulli, N. & Denaro, V. Tendon augmentation grafts: a systematic review. Br Med Bull 94, 165-188, doi:10.1093/bmb/ldp051 (2010).
37 Youngstrom, D. W. & Barrett, J. G. Engineering Tendon: Scaffolds, Bioreactors, and Models of Regeneration. Stem Cells Int 2016, 3919030, doi:10.1155/2016/3919030 (2016).
38 Gupta, A. K., Hug, K., Boggess, B., Gavigan, M. & Toth, A. P. Massive or 2-tendon rotator cuff tears in active patients with minimal glenohumeral arthritis: clinical and radiographic outcomes of reconstruction using dermal tissue matrix xenograft. Am J Sports Med 41, 872-879, doi:10.1177/0363546512475204 (2013).
39 Petri, M., Greenspoon, J. A., Moulton, S. G. & Millett, P. J. Patch-Augmented Rotator Cuff Repair and Superior Capsule Reconstruction. The open orthopaedics journal 10, 315-323, doi:10.2174/1874325001610010315 (2016).
40 Wong, I., Burns, J. & Snyder, S. Arthroscopic GraftJacket repair of rotator cuff tears. J Shoulder Elbow Surg 19, 104-109, doi:10.1016/j.jse.2009.12.017 (2010).
41 Bond, J. L., Dopirak, R. M., Higgins, J., Burns, J. & Snyder, S. J. Arthroscopic replacement of massive, irreparable rotator cuff tears using a GraftJacket allograft: technique and preliminary results. Arthroscopy 24, 403-409.e401, doi:10.1016/j.arthro.2007.07.033 (2008).
42 Gupta, A. K. et al. Dermal tissue allograft for the repair of massive irreparable rotator cuff tears. Am J Sports Med 40, 141-147, doi:10.1177/0363546511422795 (2012).
43 Thangarajah, T. et al. Augmentation of Rotator Cuff Repair With Soft Tissue Scaffolds. Orthop J Sports Med 3, 2325967115587495, doi:10.1177/2325967115587495 (2015).
44 Gilbert, T. W., Freund, J. M. & Badylak, S. F. Quantification of DNA in biologic scaffold materials. J Surg Res 152, 135-139, doi:10.1016/j.jss.2008.02.013 (2009).
45 Roth, S. P., Erbe, I. & Burk, J. Decellularization of Large Tendon Specimens: Combination of Manually Performed Freeze-Thaw Cycles and Detergent Treatment. Methods Mol Biol 1577, 227-237, doi:10.1007/7651_2017_49 (2018).
46 Kryger, G. S. et al. A comparison of tenocytes and mesenchymal stem cells for use in flexor tendon tissue engineering. J Hand Surg Am 32, 597-605, doi:10.1016/j.jhsa.2007.02.018 (2007).
47 Ozasa, Y., Amadio, P. C., Thoreson, A. R., An, K. N. & Zhao, C. Repopulation of intrasynovial flexor tendon allograft with bone marrow stromal cells: an ex vivo model. Tissue Eng Part A 20, 566-574, doi:10.1089/ten.TEA.2013.0284 (2014).
48 Farnebo, S. et al. Design and characterization of an injectable tendon hydrogel: a novel scaffold for guided tissue regeneration in the musculoskeletal system. Tissue Eng Part A 20, 1550-1561, doi:10.1089/ten.TEA.2013.0207 (2014).
49 Zhou, S., Eid, K. & Glowacki, J. Cooperation Between TGF-β and Wnt Pathways During Chondrocyte and Adipocyte Differentiation of Human Marrow Stromal Cells. Journal of Bone and Mineral Research 19, 463-470, doi:https://doi.org/10.1359/JBMR.0301239 (2004).
50 Shukunami, C. et al. Scleraxis is a transcriptional activator that regulates the expression of Tenomodulin, a marker of mature tenocytes and ligamentocytes. Sci Rep 8, 3155, doi:10.1038/s41598-018-21194-3 (2018).
51 Docheva, D., Hunziker, E. B., Fässler, R. & Brandau, O. Tenomodulin is necessary for tenocyte proliferation and tendon maturation. Mol Cell Biol 25, 699-705, doi:10.1128/mcb.25.2.699-705.2005 (2005).
52 Deprés-Tremblay, G. et al. Rotator cuff repair: a review of surgical techniques, animal models, and new technologies under development. J Shoulder Elbow Surg 25, 2078-2085, doi:10.1016/j.jse.2016.06.009 (2016).
53 Derwin, K. A., Baker, A. R., Codsi, M. J. & Iannotti, J. P. Assessment of the canine model of rotator cuff injury and repair. J Shoulder Elbow Surg 16, S140-148, doi:10.1016/j.jse.2007.04.002 (2007).
54 Liu, X., Manzano, G., Kim, H. T. & Feeley, B. T. A rat model of massive rotator cuff tears. J Orthop Res 29, 588-595, doi:10.1002/jor.21266 (2011).
55 Carpenter, J. E., Thomopoulos, S. & Soslowsky, L. J. Animal models of tendon and ligament injuries for tissue engineering applications. Clin Orthop Relat Res, S296-311, doi:10.1097/00003086-199910001-00029 (1999).
56 Lebaschi, A. et al. Animal models for rotator cuff repair. Ann N Y Acad Sci 1383, 43-57, doi:10.1111/nyas.13203 (2016).
57 Wang, C., Hu, Q., Song, W., Yu, W. & He, Y. Adipose Stem Cell-Derived Exosomes Decrease Fatty Infiltration and Enhance Rotator Cuff Healing in a Rabbit Model of Chronic Tears. Am J Sports Med 48, 1456-1464, doi:10.1177/0363546520908847 (2020).
58 Sun, Y. et al. Remnant Tendon Preservation Enhances Rotator Cuff Healing: Remnant Preserving Versus Removal in a Rabbit Model. Arthroscopy 36, 1834-1842, doi:10.1016/j.arthro.2020.03.012 (2020).
59 Vaishnav, S. & Millett, P. J. Arthroscopic rotator cuff repair: scientific rationale, surgical technique, and early clinical and functional results of a knotless self-reinforcing double-row rotator cuff repair system. J Shoulder Elbow Surg 19, 83-90, doi:10.1016/j.jse.2009.12.012 (2010).
60 Cole, B. J. et al. Arthroscopic rotator cuff repair: prospective functional outcome and repair integrity at minimum 2-year follow-up. J Shoulder Elbow Surg 16, 579-585, doi:10.1016/j.jse.2006.12.011 (2007).
61 Toussaint, B., Schnaser, E., Bosley, J., Lefebvre, Y. & Gobezie, R. Early structural and functional outcomes for arthroscopic double-row transosseous-equivalent rotator cuff repair. Am J Sports Med 39, 1217-1225, doi:10.1177/0363546510397725 (2011).
62 Park, M. C., Elattrache, N. S., Ahmad, C. S. & Tibone, J. E. “Transosseous-equivalent” rotator cuff repair technique. Arthroscopy 22, 1360.e1361-1365, doi:10.1016/j.arthro.2006.07.017 (2006).
63 Mook, W. R., Greenspoon, J. A. & Millett, P. J. Arthroscopic Double-Row Transosseous Equivalent Rotator Cuff Repair with a Knotless Self-Reinforcing Technique. Open Orthop J 10, 286-295, doi:10.2174/1874325001610010286 (2016).
64 Gigante, A., Bevilacqua, C., Ricevuto, A., Mattioli-Belmonte, M. & Greco, F. Membrane-seeded autologous chondrocytes: cell viability and characterization at surgery. Knee Surgery, Sports Traumatology, Arthroscopy 15, 88-92, doi:10.1007/s00167-006-0115-9 (2007).
65 Bunker, D. L., Ilie, V., Ilie, V. & Nicklin, S. Tendon to bone healing and its implications for surgery. Muscles Ligaments Tendons J 4, 343-350 (2014).
66 Gresham, R. C. H., Bahney, C. S. & Leach, J. K. Growth factor delivery using extracellular matrix-mimicking substrates for musculoskeletal tissue engineering and repair. Bioact Mater 6, 1945-1956, doi:10.1016/j.bioactmat.2020.12.012 (2021).
67 Porzionato, A. et al. Tissue-Engineered Grafts from Human Decellularized Extracellular Matrices: A Systematic Review and Future Perspectives. Int J Mol Sci 19, doi:10.3390/ijms19124117 (2018).
68 Chen, C. et al. Functional decellularized fibrocartilaginous matrix graft for rotator cuff enthesis regeneration: A novel technique to avoid in-vitro loading of cells. Biomaterials 250, 119996, doi:https://doi.org/10.1016/j.biomaterials.2020.119996 (2020).

1. A decellularized tendon matrix (DTM) composition comprising matrix metalloproteinase (MMP) digested tendon tissue, wherein when formulated in a formulation comprising an amount of fluid and about 1 g of composition, the formulation has a complex modulus plateau between about 10³ Pa and about 10^{6.5} Pa.

2. The composition of claim 1, wherein the amount of fluid is between about 1 ml and about 7 ml.

3. The composition of claim 1 or claim 2, wherein when formulated in a formulation comprising about 1 ml of fluid and about 1 g of composition, the formulation has a complex modulus plateau between about 10⁵ Pa and about 10^{6.5} Pa.

4. The composition of claim 1 or claim 2, wherein when formulated in a formulation comprising about 3 ml of fluid and about 1 g of composition, the formulation has a complex modulus plateau between about 10^{4.5} Pa and about 10^{5.5} Pa.

5. The composition of claim 1 or claim 2, wherein when formulated in a formulation comprising about 3 ml of fluid and about 1 g of composition, the formulation has a complex modulus plateau between about 10⁴ Pa and about 10⁵ Pa.

6. The composition of claim 1 or claim 2, wherein when formulated in a formulation comprising about 5 ml of fluid and about 1 g of composition, the formulation has a complex modulus plateau between about 10^{3.5} Pa and about 10⁵ Pa.

7. The composition of claim 1 or claim 2, wherein when formulated in a formulation comprising between about 3 ml and about 5 ml of fluid and about 1 g of composition, the formulation has a complex modulus plateau between about 10^{3.5} Pa and about 10^{5.5} Pa.

8. The composition of claim 1 or claim 2, wherein when formulated in a formulation comprising about 7 ml of fluid and about 1 g of composition, the formulation has a complex modulus plateau between about 10³ Pa and about 10^{4.5} Pa.

9. The composition of claim 1 or claim 2, wherein when formulated in a formulation comprising between about 3 ml and about 7 ml of fluid and about 1 g of composition, the formulation has a complex modulus plateau between about 10³ Pa and about 10^{5.5} Pa.

10. The composition of claim 1 or claim 2, wherein when formulated in a formulation comprising between about 1 ml and about 7 ml of fluid and about 1 g of composition, the formulation has a complex modulus plateau between about 10³ Pa and about 10^{6.5} Pa.

11. The composition of claim 1 or claim 2, wherein when formulated in a formulation comprising about 1 ml of fluid and about 1 g of composition, the formulation has a storage modulus between about 10⁵ Pa and about 10^{6.5} Pa at an angular frequency between about 10⁻¹ rad/s and about 10² rad/s.

12. The composition of claim 1 or claim 2, wherein when formulated in a formulation comprising about 1 ml of fluid and about 1 g of composition, the formulation has a loss modulus between about 10^{4.5} Pa and about 10⁶ Pa at an angular frequency between about 10⁻¹ rad/s and about 10² rad/s.

13. The composition of claim 1 or claim 2, wherein when formulated in a formulation comprising about 3 ml of fluid and about 1 g of composition, the formulation has a storage modulus between about 10⁴ Pa and about 10^{5.5} Pa at an angular frequency between about 10⁻¹ rad/s and about 10² rad/s.

14. The composition of claim 1 or claim 2, wherein when formulated in a formulation comprising about 3 ml of fluid and about 1 g of composition, the formulation has a loss modulus between about 10^{3.5} Pa and about 10⁵ Pa at an angular frequency between about 10⁻¹ rad/s and about 10² rad/s.

15. The composition of claim 1 or claim 2, wherein when formulated in a formulation comprising about 5 ml of fluid and about 1 g of composition, the formulation has a storage modulus between about 10³ Pa and about 10^{5.5} Pa at an angular frequency between about 10⁻¹ rad/s and about 10² rad/s.

16. The composition of claim 1 or claim 2, wherein when formulated in a formulation comprising about 5 ml of fluid and about 1 g of composition, the formulation has a loss modulus between about 10³ Pa and about 10⁵ Pa at an angular frequency between about 10⁻¹ rad/s and about 10² rad/s.

17. The composition of claim 1 or claim 2, wherein when formulated in a formulation comprising about 7 ml of fluid and about 1 g of composition, the formulation has a storage modulus between about 10^{2.5} Pa and about 10^{4.5} Pa at an angular frequency between about 10⁻¹ rad/s and about 10² rad/s.

18. The composition of claim 1 or claim 2, wherein when formulated in a formulation comprising about 7 ml of fluid and about 1 g of composition, the formulation has a loss modulus between about 10² Pa and about 10⁴ Pa at an angular frequency between about 10⁻¹ rad/s and about 10² rad/s.

19. The composition of claim 1 or claim 2, wherein when formulated in a formulation comprising about 3 ml of fluid and about 1 g of composition, the formulation has a storage modulus between about 10^{3.5} Pa and about 10^{5.5} Pa at an angular frequency between about 10⁻¹ rad/s and about 10² rad/s.

20. The composition of claim 1 or claim 2, wherein when formulated in a formulation comprising about 3 ml of fluid and about 1 g of composition, the formulation has a loss modulus between about 10³ Pa and about 10⁵ Pa at an angular frequency between about 10⁻¹ rad/s and about 10² rad/s.

21. The composition of any one of claims 1 to 20, wherein the formulation has a phase angle plateau between about 5° and about 25°.

22. The composition of any one of claims 1 to 21, wherein the formulation has a yield stress between about 50 Pa and about 12,500 Pa.

23. The composition of any one of claims 1 to 22, wherein the formulation is aged between about 5 minutes and about 60 minutes before measuring any one of storage modulus, loss modulus, complex modulus, phase angle, and/or yield stress.

24. The composition of any one of claims 1 to 23, wherein the fluid is phosphate-buffered saline (PBS).

25. The composition of any one of claims 1 to 24, wherein the tendon tissue is of human or animal origin.

26. The composition of any one of claims 1 to 25, wherein the composition is made by a method comprising a decellularizing step comprising contacting the tendon tissue with a DNase solution.

27. The composition of claim 26, wherein the method further comprises contacting a decellularized tendon tissue with a metalloproteinase (MMP) solution.

28. The composition of claim 26 or claim 27, wherein the method further comprises a lyophilizing step.

29. The composition of any one of claims 26 to 28, wherein the method further comprises a reconstituting step.

30. The composition of any one of claims 26 to 29, wherein the method further comprises a washing step.

31. The composition of any one of claims 26 to 30, wherein the method further comprises a filtering step.

32. The composition of any one of claims 1 to 31, wherein the MMP comprises a collagenase.

33. A method of modulating the storage modulus, loss modulus, and/or complex modulus of the formulation comprising the composition of any one of claims 1 to 32, the method comprising aging the formulation for a period of time.

34. The method of claim 33, wherein the period of time is about 30 minutes.

35. The method of claim 33 or claim 34, wherein the modulation of the storage modulus, loss modulus, and/or complex modulus is within one order of magnitude.

36. A method of stimulating cartilage regeneration, bone regeneration, or tendon enthesis regeneration in a subject in need thereof, the method comprising administering to the subject the composition of any one of claims 1 to 32 or any formulation thereof.

37. A method of stimulating angiogenesis in a cartilage tissue, a bone tissue, or enthesis tissue in a subject in need thereof, the method comprising administering to the subject the composition of any one of claims 1 to 32 or any formulation thereof.

38. The method of claim 36 or claim 37, wherein the formulation comprises a lyophilized form of the composition and a fluid.

39. The method of claim 38, wherein the ratio of fluid to the composition is between about 1 ml and about 7 ml of fluid to about 1 g of composition.

40. The method of claim 38 or claim 39, wherein the fluid is phosphate-buffered saline (PBS).
